(19) United States
(12) Patent Application Publication
(10) Pub. No.: US 2024/0376533 A1
(43) Pub. Date: Nov. 14, 2024

(54) MEASUREMENT OF NUCLEIC ACID VARIANTS USING HIGHLY-MULTIPLEXED ERROR-SUPPRESSED DEEP SEQUENCING

(71) Applicant: Abhijit Ajit PATEL, Madison, CT (US)

(72) Inventor: Abhijit Ajit PATEL, Madison, CT (US)

(21) Appl. No.: 18/416,038

(22) Filed: Jan. 18, 2024

Related U.S. Application Data
(63) Continuation of application No. 17/173,006, filed on Feb. 10, 2021, now abandoned.
Application No. 17/173,006 is a continuation of application No. 15/863,179, filed on Jan. 5, 2018, now abandoned.
Application No. 15/863,179 is a continuation of application No. 14/384,581, filed on Sep. 11, 2014, now Pat. No. 9,862,995.
(60) Provisional application No. 61/609,985, filed on Mar. 13, 2012.

(51) Int. Cl. C12Q 1/6853; C12Q 1/6806; C12Q 1/6858

(52) U.S. Cl. CPC C12Q 1/6853; C12Q 1/6806; C12Q 1/6858

(57) ABSTRACT

Methods and compositions are disclosed for measuring low-abundance DNA variants from a complex mixture of DNA molecules. Embodiments of the methods allow for extremely sensitive detection and can distinguish true variants from sequencer misreads and PCR misincorporations.

### CROSS REFERENCE TO RELATED APPLICATIONS

This application is a continuation application of U.S. Nonprovisional patent application Ser. No. 17/173,006, filed on Feb. 10, 2021, which is a continuation of U.S. Nonprovisional patent application Ser. No. 15/863,179, filed on Jan. 5, 2018, which is a continuation of U.S. Nonprovisional patent application Ser. No. 14/384,581, filed on Sep. 11, 2014, which is a U.S. National Stage Entry Application under 35 U.S.C. § 371 (b) of International Patent Application Serial No. PCT/US2013/031014, filed Mar. 13, 2013, which claims priority to U.S. Provisional Patent Application Ser. No. 61/609,985, filed on Mar. 13, 2012, the disclosures of all of which are hereby expressly incorporated by reference in their entireties.

### GOVERNMENTAL INTERESTS

The research leading to this application was funded by the National Institutes of Health from grant RR014139. The government has certain rights in this invention.

### SEQUENCE LISTING

This application contains a sequence listing, which is incorporated herein by reference in ST.26 XML format entitled “SeqList_18_416038_ST_26_V2.xml”, created Apr. 23, 2024, and is 475 KB in size. The sequences contained in the sequence listing are found throughout the originally filed application, including Tables 1-2, 5, 7-11, 13.

### BACKGROUND

Tumor-derived DNA is released into the bloodstream from dying cancer cells in patients with various types of malignancies. Such circulating tumor DNA (ctDNA) is showing excellent promise as a non-invasive cancer biomarker. However, an assay that is capable of exploiting ctDNA for early cancer detection presents several challenges. In the bloodstream, ctDNA can be distinguished from normal background DNA based on the presence of tumor-specific mutations. However, mutant ctDNA is usually only present in small amounts, having been previously reported to comprise an average of 0.2% of total plasma DNA (Diehl et al., Nat Med. 2008; 14: 985-990). If variant DNA sequences are low in abundance, detecting and quantifying these variants can be more challenging. Small amounts of mutation-harboring ctDNA can be obscured by a relative excess of background wild-type plasma DNA. Thus, an assay with extremely high detection sensitivity is required.

There is a need for a method that is able to detect and quantify rare variant sequences to detect cancers in situations where the amount of DNA in a given sample is limited. Unlike existing approaches, a test should be able to evaluate an entire panel of mutation-prone regions without needing to divide DNA samples into separate reactions (which could reduce detection sensitivity by providing fewer template DNA copies per reaction). Methods and compositions are described herein that provide a multiplex assay to detect minute amounts of ctDNA and address the current deficiencies to assay ctDNA.

### SUMMARY

Described herein are compositions and methods relating to next-generation sequencing and medical diagnostics. Methods include identifying and quantifying nucleic acid variants, particularly those available in low abundance or those obscured by an abundance of wild-type sequences. Also described herein are methods related to identifying and quantifying specific sequences from a plurality of sequences amid a plurality of samples. Methods as described herein also include detecting and distinguishing true nucleic acid variants from misincorporation errors, sequencer errors, and sample misclassification errors. Methods include early attachment of barcodes and molecular lineage tags (MLTs) to targeted nucleic acids within a sample. Methods also include clonal overlapping paired-end sequencing to achieve sequence redundancy.

In an embodiment, a method includes measuring nucleic acid variants by tagging and amplifying low abundance template nucleic acids in a multiplexed primer extension or PCR. Low abundance template nucleic acids may be fetal DNA, circulating tumor DNA (ctDNA), viral RNA, viral DNA, DNA from a rejected transplanted organ, or bacterial DNA. A multiplex PCR may include gene specific primers, wherein primers are specific for a mutation prone region (e.g., within KRAS, EGFR, etc.). In an embodiment, a mutation prone region may be associated with cancer. As disclosed herein, a multiplex PCR can include more than one round of PCR and/or primer-extension. In an embodiment, a multiplex PCR can include two or three rounds of PCR.

In an embodiment, primers comprise a barcode and/or a molecular lineage tag (MLT). In an embodiment, an MLT can be 2-10 nucleotides. In another embodiment, an MLT can be 6, 7, or 8 nucleotides. In an embodiment, a barcode can identify the sample of template nucleic acid. In an embodiment, a PCR reaction mixture includes template nucleic acids from multiple samples (e.g., patients), wherein the barcode identifies the sample origin of the template nucleic acid. In an embodiment, a primer extension reaction employs targeted early barcoding. In targeted early barcoding, a plurality of different primers specific for different nucleic acid regions all have an identical barcode. An identical barcode identifies the nucleic acids from a particular sample. In an embodiment, primers used for targeted early barcoding are produced by combining a unique barcode-containing oligonucleotide segment with a uniform mixture of gene-specific primer segments in a modular fashion.

In an embodiment, multiplex assays described herein can be used for clinical purposes. In an embodiment, nucleic acid variants within blood can be identified and measured before and after treatment. In an example of cancer, a nucleic acid variant (e.g., cancer-related mutation) can be identified and/or measured prior to treatment (e.g., chemotherapy, radiation therapy, surgery, biologic therapy, combinations thereof). Then after treatment, the same nucleic acid variant can be identified or measured. After treatment, a decrease or absence of the nucleic acid variant can indicate that the therapy was successful.

### BRIEF DESCRIPTION OF THE DRAWINGS

FIG. 1 is a schematic of a copying, tagging, amplification, and sequencing process. Two rounds of limited-cycle PCR were performed to attach barcode sequences and molecular lineage tag sequences to copies of targeted template DNA fragments. Stringent purification of the PCR products was carried out between rounds in order to remove unextended primers, spurious extension products, and template DNA. A final round of PCR was performed using universal primers to further amplify the purified products from Round 2. Final amplification products were gel-purified and subjected to clonal overlapping paired-end massively parallel sequencing. Use of primers synthesized from modular segments allowed early barcoding of targeted template DNA during the first round of PCR, enabling subsequent steps to be performed in a combined reaction volume.

FIG. 2 illustrates a general approach to combining modular oligonucleotide segments to produce mixtures of gene-specific barcoded primers. Primers produced by combining modular segments allow primer-extension and early barcoding of multiple targeted gene regions in a given sample. A primer mix used for a particular sample may have a unique barcode and multiple gene-specific primer sequences. For a different sample, the mix can have the same set of gene-specific sequences in identical ratios, but a different barcode.

FIG. 3 illustrates a method for producing combinations of modular oligonucleotide segments using an automated oligonucleotide synthesizer. First, gene-specific 3′-segments of oligonucleotides were synthesized on solid supports on separate synthesis columns. The oligonucleotides were synthesized in a 3′ to 5′ direction. The synthesis was then paused, and the partially-synthesized oligonucleotides were left in a protected state on solid support particles. The contents of all columns were evenly mixed, and the mixture of solid support particles was then dispensed into separate fresh columns. Synthesis of the barcode-containing 5′-segment of the oligonucleotides was then continued in the new columns. A uniquely barcoded 5′-segment was added in each column. After cleavage, deprotection and purification, the resulting barcoded oligonucleotide mixtures all had identical ratios of 3′-segments.

FIGS. 4A and FIG. 4B are schematics of error-suppressed multiplexed deep sequencing. FIG. 4A shows cell-free DNA purified from plasma undergoing two rounds of amplification by PCR. The first round amplifies mutation hotspot regions of several genes from a given sample in a single tube. The second round separately amplifies each hotspot region using nested primers incorporating unique combinations of barcodes to label distinct samples. The barcoded PCR products are then pooled and subjected to deep sequencing. Millions of sequences are sorted and counted to determine the ratio of mutant to wild-type molecules derived from each sample. The total number of plasma DNA fragments is measured by real-time PCR and can be used to calculate the absolute concentration of mutant ctDNA. FIG. 4B shows sequence redundancy in mutation hotspot regions is produced by partial overlap of paired-end reads from the forward and reverse strands of each clone. This yields highly accurate base-calls, permitting detection and quantitation of rare mutations with greater sensitivity.

FIGS. 5A-C are graphs depicting suppression of spurious mutation counts to reveal low-abundance variants. Each bar indicates the frequency of a particular deviation from the wild-type sequence occurring within the codon 12/13 hotspot region of KRAS. The tested sample contains 0.2% DNA derived from a lung cancer cell line that is known to be homozygous for a KRAS Gly12Ser mutation. FIG. 5A shows filtered reads from one end of the amplicon have relatively frequent mismatches when directly compared to the wild-type sequence. Data from 3 replicate amplifications are shown. FIG. 5B indicates sequencer errors are greatly reduced by requiring both partially overlapping paired-end reads from each clone to exactly match a specific mutation. The Gly12Ser mutation is now readily distinguished from the remaining low-level errors that were likely introduced during DNA amplification and processing. Insertions and deletions are no longer seen in this region after requiring agreement of overlapped reads. FIG. 5C shows a further reduction in the relative error level can be achieved by calculating the mean values of 3 replicate measurements, since mutations found in the original DNA sample should produce more consistent counts than randomly occurring errors.

FIGS. 6A-C indicates the performance of error-suppressed deep sequencing.

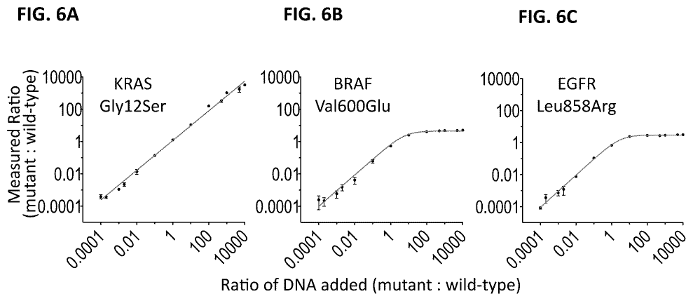

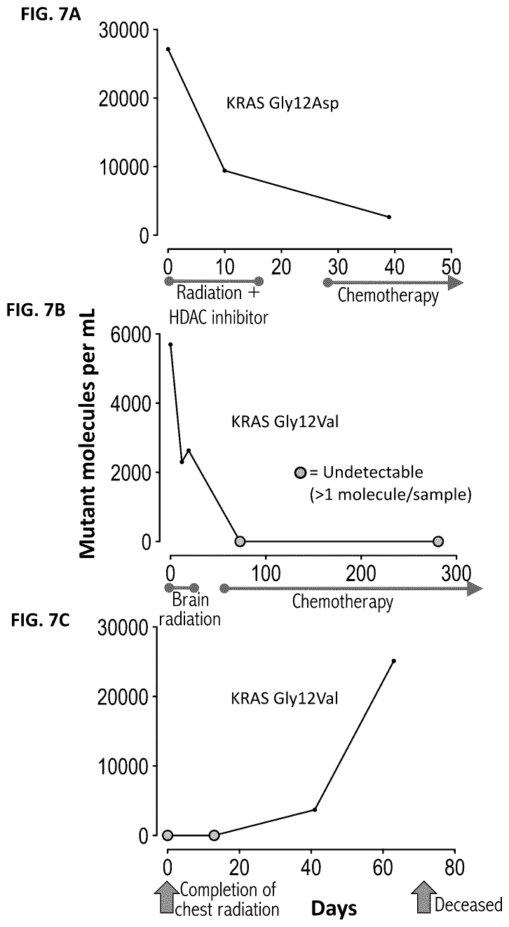

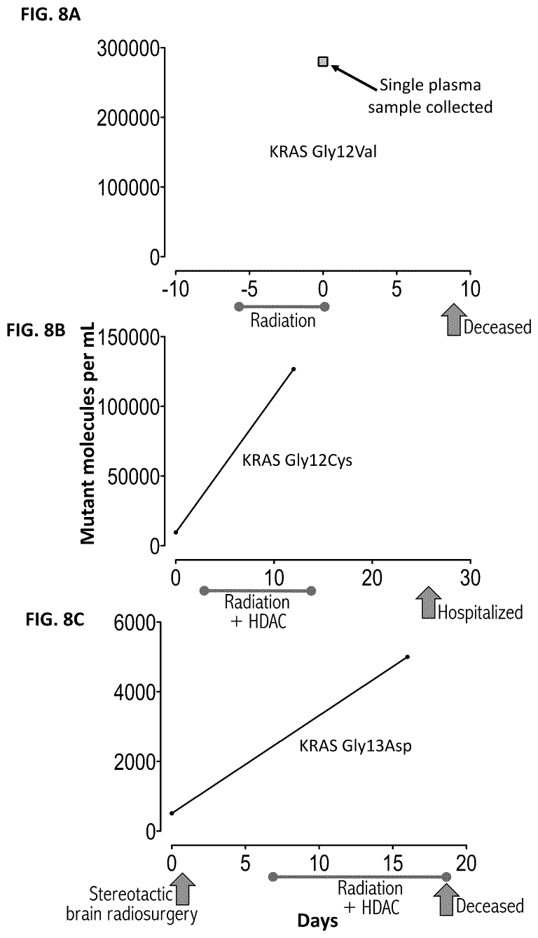

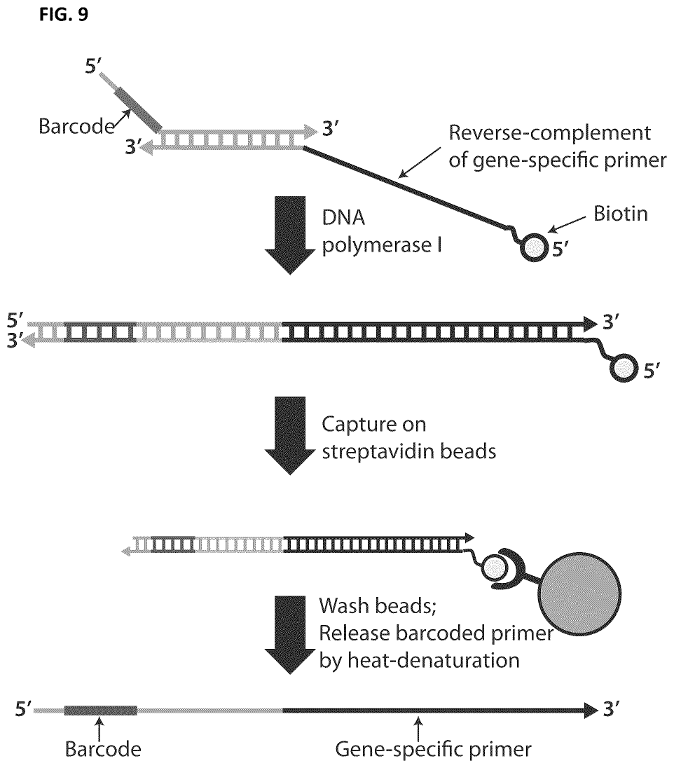

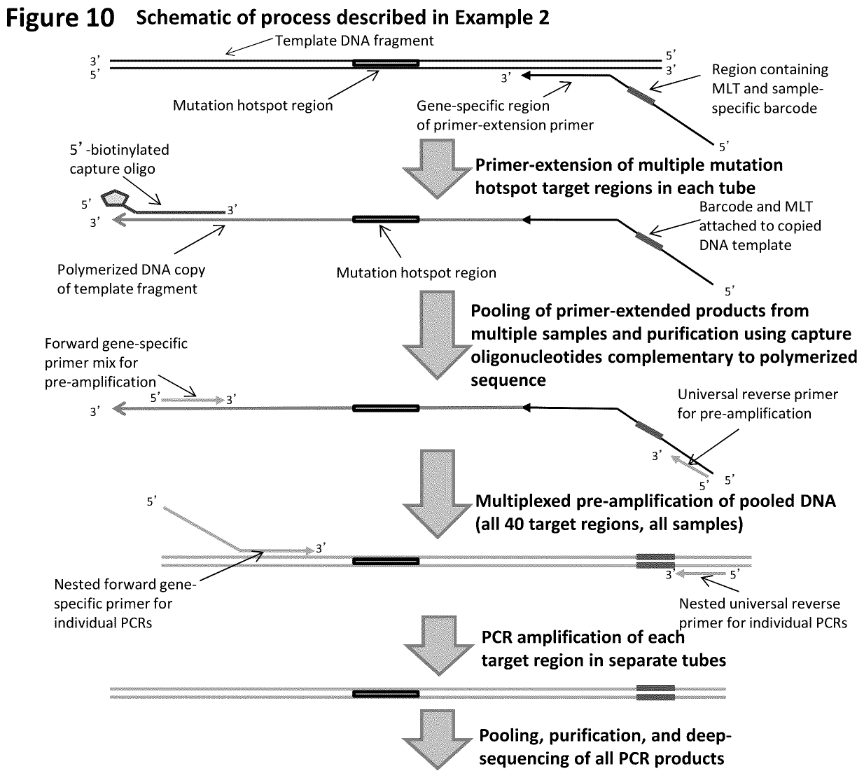

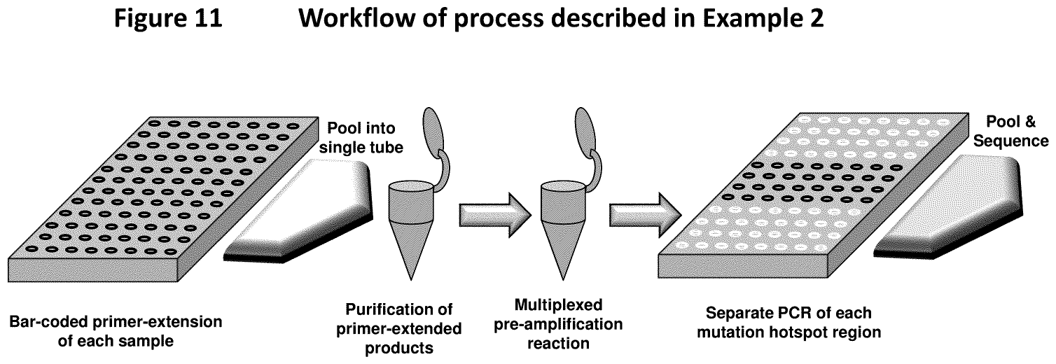

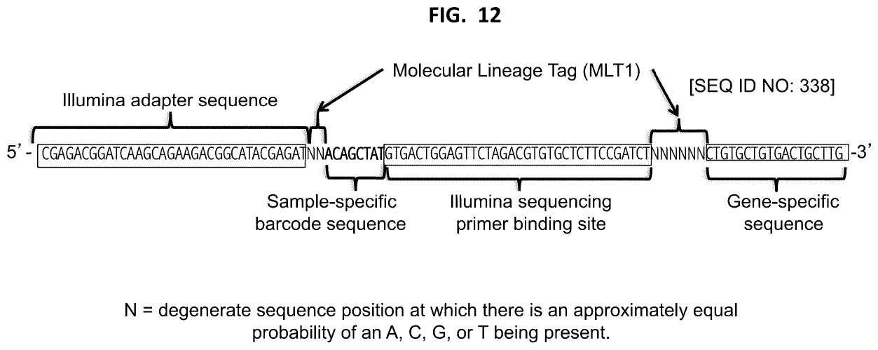

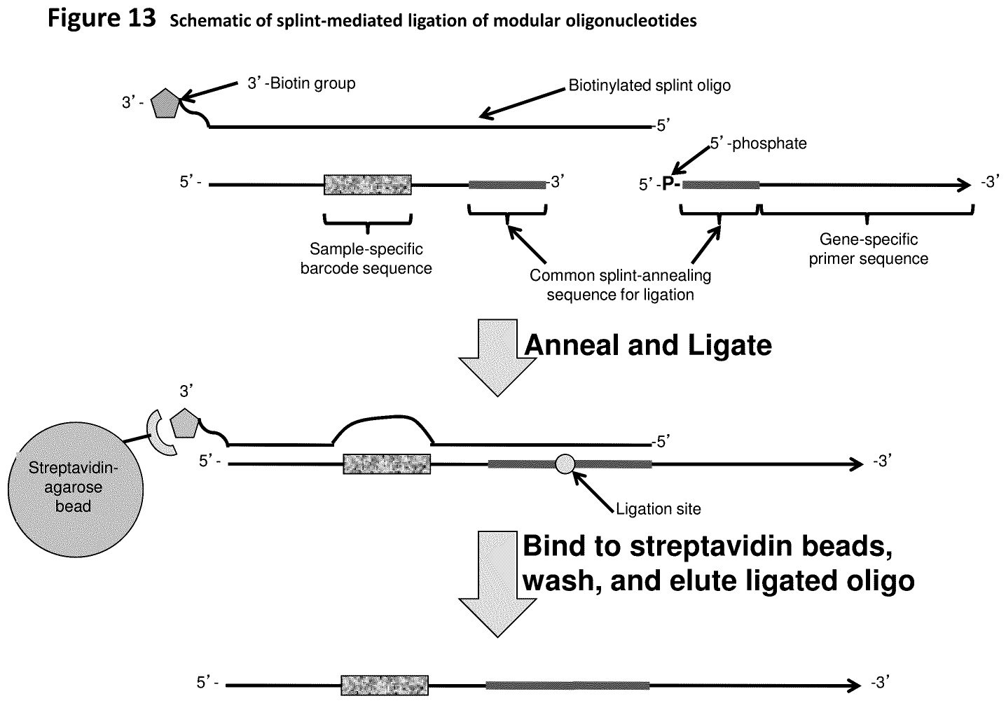

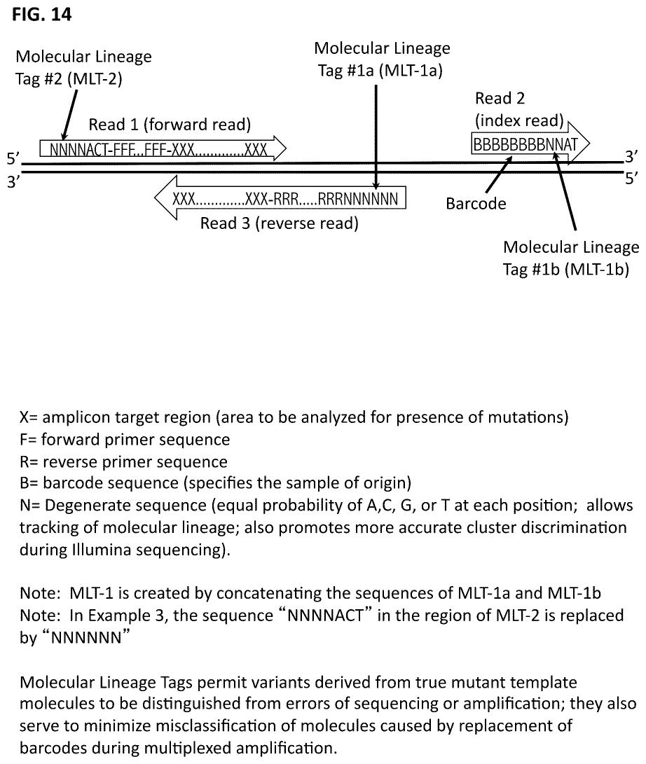

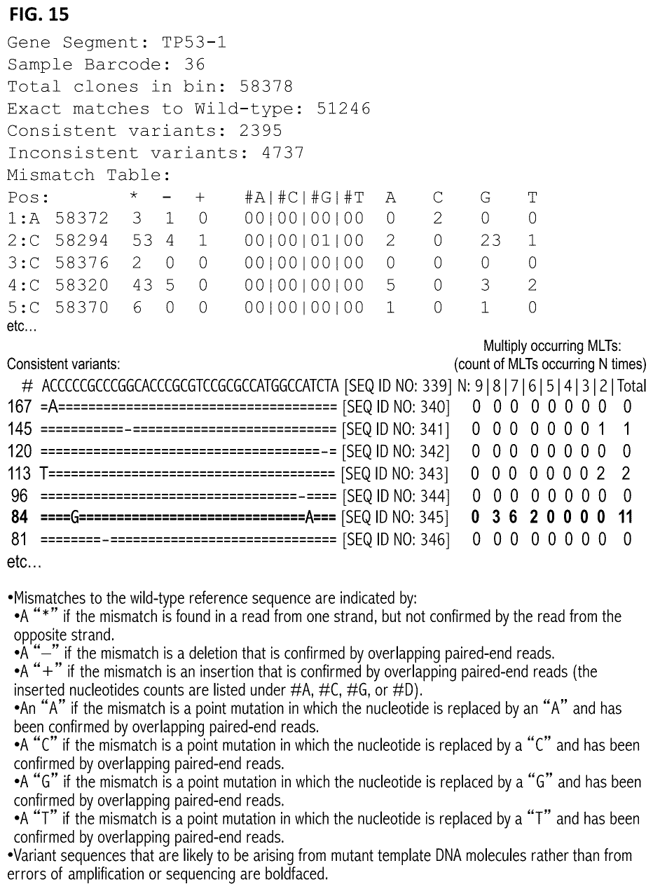

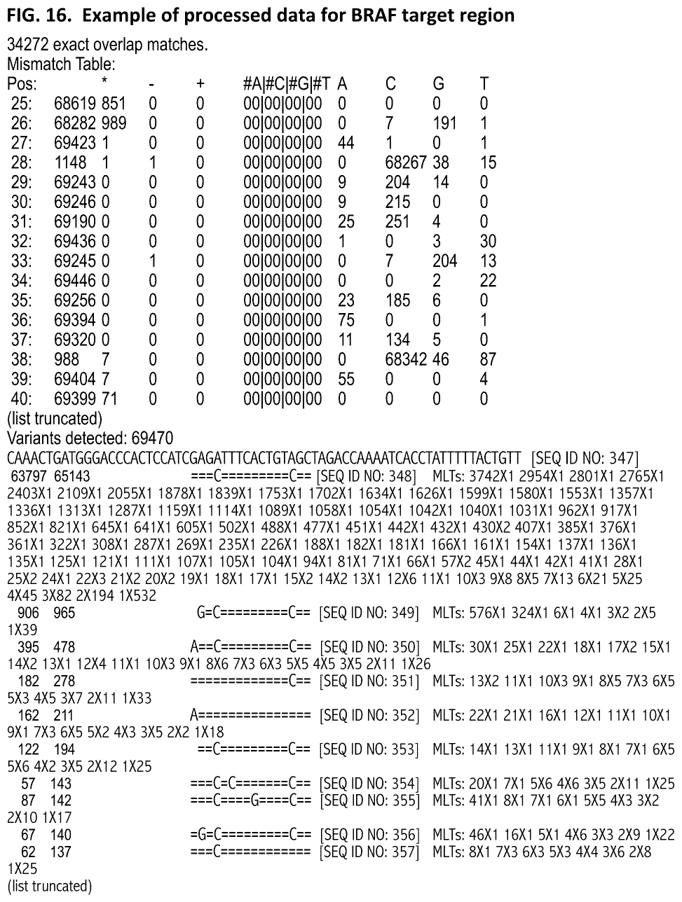

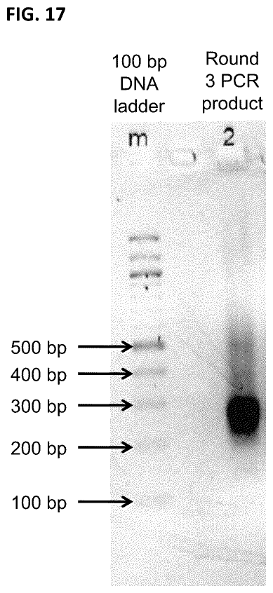

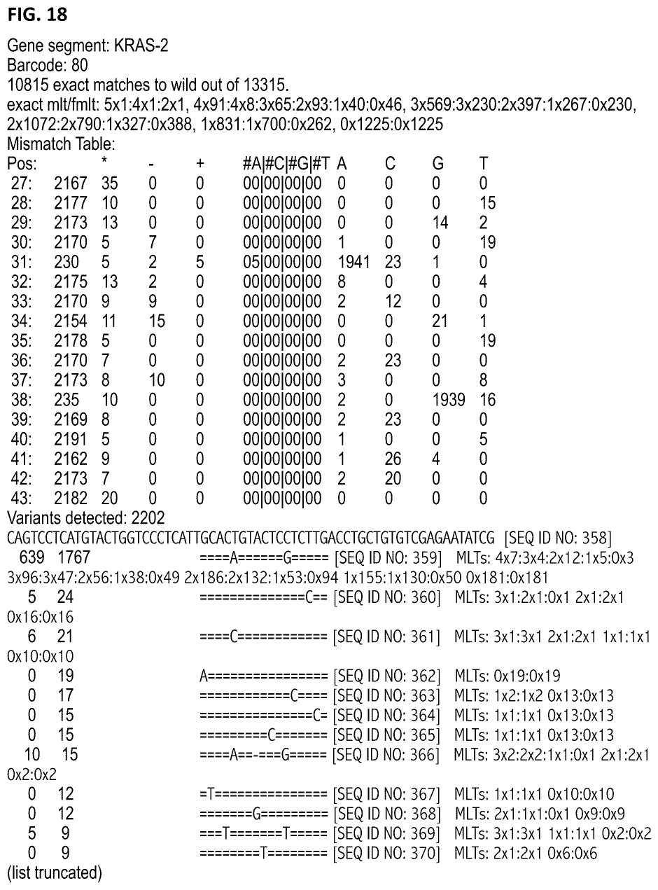

Measurements of DNA extracted from mutant and wild-type cancer cell lines mixed in various ratios ranging from 1:10,000 to 10,000:1 show a high degree of accuracy and reproducibility. FIG. 6A is a linear plot of DNA from the KRAS-mutant cell line over the range of concentrations tested. FIG. 6B and FIG. 6C show that BRAF-and EGFR-mutant lines, respectively, contained a small amount of wild-type DNA, thereby yielding a plateau at higher mutant to wild-type ratios. Non-linear least-squares fits were performed using the equation y=10\hat{ }(slope*log((1−C)*x/(C*x+1))+intercept) where C was the fraction of wild-type molecules found in DNA extracted from mutant cell lines. Error bars indicate the standard deviation of 3 measurements.

FIGS. 7A-C show the changes in ctDNA levels with treatment or disease progression. Measurements of mutant ctDNA from patients with NSCLC are shown at various times in relation to therapeutic interventions and disease status. ctDNA was considered undetectable if sequence counts yielded a quantity of less than one mutant molecule per sample. Median genome equivalents per sample, as determined by real-time PCR were 9602 (IQR =5412-11513) FIG. 7A shows the timeline of treatment for Patient 3 who had stage IV lung adenocarcinoma with a 4.3cm right upper lobe tumor and large metastases in the abdomen and supraclavicular region. She was treated concurrently with an experimental histone deacetylase (HDAC) inhibitor and palliative radiation therapy directed at her painful 6.9 cm supraclavicular lesion. She began chemotherapy treatment shortly afterwards. FIG. 7B shows the timeline of treatment for Patient 5 who had a 7.5 cm lung adenocarcinoma with eight small brain metastases ranging from 3 mm to 15 mm in size at presentation. He was treated with palliative whole-brain radiation therapy, followed by long-term weekly chemotherapy. Follow-up imaging revealed an excellent, durable response with shrinkage of the lung tumor to ˜15% of its original volume at 7 months after diagnosis. No evidence of disease progression was seen during this time period. FIG. 7C shows the timeline of treatment for Patient 9 who underwent definitive radiation treatment for locally advanced, stage IIIB undifferentiated NSCLC. Other health conditions prevented him from undergoing surgery or concurrent chemotherapy. Blood sample collection commenced upon completion of his treatment. Although his disease was confined to the thorax prior to initiating radiation therapy, a PET scan performed 8 weeks after treatment showed marked progression of disease with multiple osseous, hepatic, and subcutaneous metastases. He expired 10 weeks after completing treatment.

FIGS. 8A-C show the ctDNA levels in patients with fewer than 3 time-points. FIG. 8A shows the timeline of treatment for Patient 11 who had stage IV lung adenocarcinoma with widespread metastatic disease in the bones of her spine, ribs, sternum, clavicle, humerus, and pelvis. She was treated with a short course of palliative radiation therapy for a pathologic fracture in her lumbar spine. A single blood sample was obtained on her last day of treatment. She passed away approximately 1 week after completion of therapy. FIG. 8B shows the timeline of treatment for Patient 14 who had stage IV lung adenocarcinoma. He received palliative radiation therapy to a painful 8.9×5.9×4.9 cm lesion in his left posterior chest wall, given concurrently with an experimental histone deacetylase (HDAC) inhibitor. He had additional metastatic lesions in his liver, kidneys, and peri-splenic region. He was hospitalized for profound weakness 10 days post-treatment, and expired shortly afterwards. FIG. 8C shows the timeline of treatment for Patient 15 who had stage IV undifferentiated NSCLC with metastasis in the supraclavicular and inguinal regions, as well as several small tumors in his brain. His brain lesions were treated with single-fraction stereotactic radiosurgery. He then began palliative radiation therapy for a painful 7.1 cm left upper lobe lung mass, which was threatening to obstruct his left mainstem bronchus. He received concurrent treatment with a HDAC inhibitor as part of a clinical trial. He passed away unexpectedly after receiving 8 of 10 planned radiation treatments.

FIG. 9 shows a scheme for appending modular barcodes to gene-specific primers. An ability to combine barcodes and gene-specific primers in a modular fashion provides flexibility to modify or expand a panel of genes or number of samples being tested. A gene-specific primer was added to the 3′-end of a barcoded oligonucleotide by polymerization on a biotinylated template. A biotin tag was used to capture a double-stranded product onto streptavidin resin. A barcoded primer was then released into solution by heat-denaturation. In a similar manner, a mixture of biotinylated templates can be used to produce a mixture of gene specific primers, all having the same barcode. Separate reactions use different barcoded oligonucleotides to produce uniquely barcoded primer mixes that can be used for targeted early barcoding.

FIG. 10 is a schematic of a process described in Example 2. First, a primer extension step was carried out using primers that assign sample-specific barcodes and MLT sequences to copied template DNA. For a given sample, multiple targeted sequences were copied using multiple gene-specific primers, all bearing the same sample-specific barcode. After stringent purification of specifically extended products, a pre-amplification step was performed in order to produce many copies of the tagged molecules. This allowed splitting of the products into different tubes for separate amplification of each target site, while ensuring that copies of the original templates are adequately sampled. The products of the final PCRs were combined and subjected to clonal overlapping paired-end deep sequencing. Nested primers were used to enhance target specificity at each step.

FIG. 11 shows a workflow of a process described in Example 2. Separate primer-extension reactions were initially carried out for each sample. Barcoded products were then be mixed into a single volume for purification and pre-amplification steps. Purified products were then split into separate tubes and underwent final single-target PCR in separate reaction volumes.

FIG. 12 shows an example of a Round I reverse primer sequence, highlighting various elements of the sequence. Note that the gene-specific sequence at the 3′-end can act as a primer for either PCR or primer-extension by a DNA polymerase. The 5′-segment contains a sample-specific barcode sequence, a Molecular Lineage Tag (MLT), as well as adapter sequences required by the next-generation sequencing platform. In this example, a gene-specific segment is specific for a mutation prone-region of TP53. For Round 1 PCR or primer-extension of a given sample, a mixture of several reverse primers would be used, all having the same sample-specific barcode sequence, and multiple different gene-specific sequences. A similar mixture, but with another barcode, would be used for Round 1 PCR or primer-extension of a different sample.

FIG. 13 is a schematic of a process of splint-mediated ligation of modular oligonucleotide segments. A 5′-segment containing a particular barcode sequence can be ligated to a mixture of 3′-segments having a variety of gene-specific primer sequences using a biotinylated splint oligonucleotide. Hybridization to the splint oligonucleotide is mediated by common annealing sequences. A 5′-phosphate is necessary on the 3′-segments to permit enzymatic ligation. The biotinylated splint can be used to capture and wash and elute the ligated products.

FIG. 14 shows elements of a sequence output using the Illumina® platform. Read 1 and read 3 are from opposite strands, and provide sequence redundancy via overlap in the mutation-prone region. This clonal redundancy allowed sequences resulting from sequencer errors to be identified and discarded, permitting greater sensitivity for detection of rare sequence variants.

FIG. 15 shows hypothetical processing of data from sequences assigned to a single gene and a single barcode. The example illustrates how analysis of variant sequences and associated molecular lineage tags can be performed.

FIG. 16 shows processed data from sequences generated using methods described in Example 2. Data are shown for a single gene and a single barcode. Symbols used in the mismatch table are as defined in FIG. 15. MLT counts associated with variant sequences are displayed in the format “N×Z” where N is the number of copies of a particular MLT sequence, and Z is the number of different MLT sequences having N copies.

FIG. 17 shows an ethidium bromide-stained 2% agarose gel containing products of Round 3 PCR. A marker lane contained a 100 base-pair ladder for size comparison. The gel shows a diffuse band containing amplified products within the expected size range, and very little spurious product migrating at a different size.

FIG. 18 shows processed data from sequences generated from the methods described in Example 3. Data are shown for a single gene and a single barcode. Results are displayed in a format similar to FIG. 15, but in this case analysis of two separate MLT sequence regions (MLT-1 and MLT-2) was performed for variant and wild-type sequences. To report these counts in a succinct format, MLT counts are binned by powers of two. For example, an MLT-1 count of 13 would be placed into bin 4 (because 2\hat{ }4 is the smallest power of 2 that is greater than or equal to 13). Thus, a report of 4×5 means that there were five instances of counts in the range of 9 to 16. Similarly, a report of 3×6 means that there were six instances of counts in the range of 5 to 8. For a given collection of MLT-1 counts, the associated MLT-2 counts were reported in a similar format, to the right of the MLT-1 counts and separated by colons. For example, 4×5:2×3:1×7 meant that among 5 sets of MLT-1 sequences occurring between 9 and 16 times, there were 3 instances of MLT-2 sequences that occurred between 3 and 4 times, and 7 instances of MLT-2sequences that occurred twice. Different MLT-1 bins were separated by a space.

### DETAILED DESCRIPTION

### Definitions

The terms “nucleic acid,” “nucleotide,” “polynucleotide,” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “base”, in its singular form, refers to a single residue within a nucleic acid molecule or to a single position within a nucleic acid sequence read.

The term “biological sample” refers to a body sample from any animal, but preferably is from a mammal, more preferably from a human. Such samples include biological fluids such as serum, plasma, vitreous fluid, lymph fluid, synovial fluid, follicular fluid, seminal fluid, amniotic fluid, milk, whole blood, urine, cerebro-spinal fluid, saliva, sputum, tears, perspiration, mucus, and tissue culture medium, as well as tissue extracts such as homogenized tissue, and cellular extracts.

As used herein, “buffer” refers to a buffered solution that resists changes in pH by the action of its acid-base conjugate components. Buffers may optionally comprise a salt such as MgCl2, MnCl2, or the like. Buffers may also optionally comprise other constituents to improve the efficiency of reverse transcription or amplification, including, but not limited to, betaine, dimethyl sulfoxide, surfactant, bovine serum albumin, etc.

The term “cDNA” refers to a complementary DNA molecule synthesized using a ribonucleic acid strand (RNA) as a template. RNA may be mRNA, tRNA, rRNA, microRNA, or another form of RNA, such as viral RNA. The cDNA may be single-stranded, double-stranded or may be hydrogen-bonded to a complementary RNA molecule as in an RNA/cDNA hybrid.

The term “polymerase chain reaction” or “PCR” refers to a procedure or technique in which minute amounts of nucleic acid, RNA and/or DNA, are amplified as described in U.S. Pat. No. 4,683,195 issued Jul. 28, 1987. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al., Cold Spring Harbor Symp. Quant. Biol., 51: 263(1987); Erlich, ed., PCR Technology, (Stockton Press, NY, 1989).

The term “reverse transcription polymerase chain reaction” or “RT-PCR” refers to the transcription of cDNA from a RNA template by the enzyme reverse transcriptase. The cDNA is then amplified by known PCR methods.

The term “primer-extension” refers to an enzymatic process whereby a primer is hybridized to a template nucleic acid strand and is polymerized using said strand as a template. Polymerization can be mediated by enzyme classes including but not limited to DNA polymerases or reverse transcriptases. Primer-extension can take place as an isolated reaction (single extension of a primer on a template), or as part of a repetitive process such as PCR.

The term “primer” refers to an oligonucleotide capable of acting as a point of initiation of synthesis along a complementary strand when conditions are suitable for synthesis of a primer extension product. The synthesizing conditions include the presence of four different deoxyribonucleotide triphosphates (dNTPs) and at least one polymerization-inducing agent such as reverse transcriptase or DNA polymerase. These are present in a suitable buffer, which may include constituents which are co-factors or which affect conditions such as pH and the like at various suitable temperatures. A primer is preferably a single strand sequence, such that amplification efficiency is optimized, but double stranded sequences can be utilized. A primer can have some sequences that are not designed to hybridize to the targeted template DNA, including sequences at the 5′-end of the primer that becomes incorporated into the amplified products. Such sequences can include universal primer binding sites to be used in subsequent amplifications, sample-specific barcodes, or molecular lineage tags. In addition to serving the purpose of copying a nucleic acid template, a primer can also be used to append labels or other sequences to the copied products. Primers and other synthetic oligonucleotides disclosed herein have undergone either polyacrylamide gel purification or reverse-phase cartridge purification unless otherwise specified. A primer can also be modified by attachment of one or more chemical moieties including but not limited to biotin, a fluorescent tag, a phosphate, or a chemically reactive group.

The term “gene-specific primer” refers to a primer that is designed to hybridize to and be extended on a particular nucleic acid target. The 3′-segment of a gene-specific primer is complementary to its targeted RNA or DNA sequence, but other portions of the primer need not be complementary to any target. The target need not be a “gene” in the strict sense of the word. Possible targets include but are not limited to genomic DNA, mitochondrial DNA, viral DNA, mRNA, microRNA, viral RNA, tRNA, rRNA, and cDNA.

The term “nested primer” refers to a primer that is designed to hybridize to a primer-extended or PCR amplified product at a position that is either entirely or partially within the target region that was flanked by the original primers. The 3′-end of a nested primer is complementary to target sequences that would not have been contained within the original primers, but rather would have been copied by extension of the original primers on the desired template. Nested primers thus provide additional specificity for copying or amplifying a desired target after an initial round of primer-extension or PCR.

The terms “reaction mixture” or “PCR reaction mixture” or “PCR master mix” refer to an aqueous solution of constituents in a PCR or RT-PCR reaction that can be constant across different reactions. An exemplary PCR reaction mixture includes buffer, a mixture of deoxyribonucleoside triphosphates, reverse transcriptase, primers, probes, and DNA polymerase. Generally, template DNA is the variable in a PCR reaction.

The terms “sequence variant” or “mutation” are used interchangeably and refer to any variation in a nucleic acid sequence including but not limited to single point-mutations, multiple point-mutations, insertions/deletions (indels), and single-nucleotide polymorphisms (SNPs). These terms are used interchangeably in this document, and it is understood that when reference is made to a method for evaluating one type of variant, it could be equally applied to evaluation of any other type of variant. The term “variant” can also be used to refer to a single molecule whose sequence deviates from a reference sequence, or a collection of molecules whose sequences all deviate from the reference sequence in the same way. Similarly, “variant” can refer to a single sequence (or read) that deviates from a reference sequence or a set of sequences that deviate from a reference sequence.

The terms “mutation-prone region” and “mutation hotspot” are used interchangeably, and refer to a sequence region of a nucleic acid obtained from a biological source that has a higher probability of being mutated than surrounding sequence regions within the same nucleic acid. In the case of tumor-derived DNA, mutation-prone regions can be found in certain cancer-related genes. The mutation-prone region can be of any length, but mutation-prone regions that are analyzed using the methods disclosed herein are less than 100 nucleotides long. A mutation can be found anywhere within a mutation-prone region.

The term “target region” refers to a region of a nucleic acid that is targeted for primer extension or PCR amplification by specific hybridization of complementary primers.

The term “clonal overlapping paired-end sequencing” refers to a massively parallel sequencing method in which paired-end reads are obtained for each clonal sequence such that portions of the two reads from opposite strands are able to cover the same region of DNA. This approach is used to reduce or suppress or distinguish sequencer-derived errors, thereby allowing base-calls to be made with greater confidence. The region of DNA that is covered by the overlapping reads is effectively read twice in opposite directions, once from each strand of the duplex. Thus, by including the mutation-prone region within the area of sequence overlap, the mutation prone region is read in one direction and then proofread in the opposite direction. Read-pairs that do not have perfect sequence consistency in the overlapping region (after obtaining a reverse-complement of one of the reads) can be attributed to sequencer error and can be discarded from the analysis. This approach greatly reduces the background of sequencer-generated errors and allows rare mutant molecules to be detected with greater sensitivity.

The terms “barcode”, “tag”, and “index” are used interchangeably and refer to a sequence of bases at certain positions within an oligonucleotide that is used to identify a nucleic acid molecule as belonging to a particular group. A barcode is often used to identify molecules belonging to a certain sample when molecules from several samples are combined for processing or sequencing in a multiplexed fashion. A barcode can be any length, but is usually between 6 and 12 bases long (need not be consecutive bases). Barcodes are usually artificial sequences that are chosen to produce a barcode set, such that each member of the set can be reliably distinguished from every other member of the set. Various strategies have been used to produce barcode sets. One strategy is to design each barcode so that it differs from every other barcode in the set at a minimum of 2 distinct positions.

The term “sample-specific barcode” refers to a barcode sequence that is assigned to molecules that are derived from a particular sample.

The term “template nucleic acid” refers to any nucleic acids that can serve as targets for primer-extension, reverse-transcription, or PCR. A template nucleic acid can be DNA or RNA. Methods described herein for analysis of DNA can also be applied to the analysis of RNA after reverse-transcribing the RNA to produce cDNA. Methods for evaluating DNA can be equally applied to the evaluation of RNA.

The terms “deep sequencing” and “ultra-deep sequencing” are used interchangeably herein and refer to approaches that use massively parallel sequencing technologies to obtain large numbers of sequences corresponding to relatively short, targeted regions of the genome. A targeted region can include, for example, an entire gene or small segment of a gene (such as a mutation hotspot). In some cases, many thousands of clonal sequences are obtained from a short targeted segment allowing identification and quantitation of sequence variants.

The term “clonal sequence” refers to a sequence that is derived from a single molecule within a sample that is subjected to massively parallel sequencing. Specifically, each clonal sequence that is generated by massively parallel sequencing is derived from a distinct DNA molecule within a sample that serves as the “input” for the sequencing workflow.

The terms “targeted early barcoding”, “early barcoding”, “attachment of early barcodes”, and “assignment of early barcodes” are used interchangeably and refer to assignment of barcodes to selected nucleic acid targets within a sample by specific hybridization and polymerization of barcode-containing primers at an early processing step. Preferably, barcode assignment occurs during the first enzymatic step that is performed after template nucleic acid molecules are purified from a biological sample. This first enzymatic step can be primer-extension, reverse-transcription, or PCR. When multiple different target sequences are to be tagged and copied from a given sample, a mixture of several different target-specific primers are used in a single reaction volume, with every primer in the mixture having the same sample-specific barcode. Separate early barcoding reactions are carried out for each sample, using similar mixtures of primers bearing distinct barcodes for each sample. Targeted early barcoding allows molecules from different samples to be combined into a single volume for all subsequent processing steps.

The term “degenerate sequence” refers to a stretch of sequence in which, within a population of nucleic acid molecules, two or more different bases can be found at each position. Most often, degenerate sequences are produced such that there is an approximately equal probability of each position having A, C, G, or T (in the case of DNA), or having A, C, G, or U (in the case of RNA). However, in certain situations, different bases can be incorporated in varying ratios at different positions, and some bases can be omitted at certain positions if desired. A degenerate sequence can be of any length.

The terms “molecular lineage tag”, “MLT”, and “MLT sequence” are used interchangeably and refer to a stretch of degenerate sequence that is contained within a synthetic oligonucleotide (e.g. a primer) and is used to assign a set of diverse sequence tags to copies of template nucleic acid molecules. A molecular lineage tag is designed to have between 2 and 10 degenerate base positions, but preferably has between 6 and 8 base positions. The bases need not be consecutive, and can be separated by constant sequences. The number of possible MLT sequences that can be generated in a population of oligonucleotide molecules is generally determined by the length of the MLT sequence and the number of possible bases at each degenerate position. For example, if an MLT is 8 bases long, and has an approximately equal probability of having A, C, G, or T at each position, then the number of possible sequences is 4\hat{ }8=65,536. A molecular lineage tag is not designed to assign a completely unique sequence tag to each molecule, but rather is designed to have a low probability of assigning any given sequence tag to a particular molecule. The greater the number of possible MLT sequences, the lower the probability of any particular sequence being assigned to a molecule. When many template molecules are copied and tagged, the same MLT sequence can be assigned to more than one template molecule. MLT sequences are used to track the lineage of molecules from initial copying through amplification, processing and sequencing. They can be used to distinguish sequences that arise from polymerase misincorporations or sequencer errors from sequences that are derived from true mutant template molecules. MLTs can also be used to distinguish sequences that have the wrong barcode assignment as a result of cross-over of barcodes during pooled amplification. Because the same MLT sequence can be assigned to more than one template molecule, meaningful analysis of MLT sequences requires first identifying variant target sequences and then analyzing the distribution of MLT sequences associated with those variants.

The term “molecular lineage tagging” refers to the process of assigning molecular lineage tags to nucleic acid templates molecules. MLTs can be incorporated within primers, and are attached to copies made from targeted nucleic acids by specific extension of primers on the templates.

The term “include” and its derivations should be understood to mean “including, but not limited to”. The words “a”, “an”, and “the” include both singular and plural referents unless the context indicates otherwise.

### Embodiments of the Methods

Methods and compositions are disclosed herein for identifying and quantifying nucleic acid sequence variants. Methods disclosed herein can identify and quantify low-abundance sequence variants from complex mixtures of DNA or RNA. Embodiments of the methods can measure small amounts of tumor-derived DNA that can be found in the circulation of patients with various types of cancer.

Assessment of rare variant DNA sequences is important in many areas of biology and medicine. Small amounts of fetal DNA can be found in the circulation of pregnant women. An embodiment includes analyzing rare fetal DNA that can be used to assess disease-associated genetic features or the sex of the fetus. An organ that is undergoing rejection by the recipient can release small amounts of DNA into the blood, and this donor-derived DNA can be distinguished based on genetic differences between the donor and the recipient. An embodiment includes measuring donor-derived DNA to provide information about organ rejection and efficacy of treatment. In another embodiment, nucleic acids can be detected from an infectious agent (e.g., bacteria, virus, fungus, parasite, etc.) in a patient sample. Genetic information about variations in pathogen nucleic acids can help to better characterize the infection and to guide treatment decisions. For instance, detection of antibiotic resistance genes in the bacterial genome infecting a patient can direct antibiotic treatments.

Detection and measurement of low-abundance mutations has many important applications in the field of oncology. Since tumors are known to acquire somatic mutations, some of which promote the unregulated proliferation of cancer cells, identifying and quantifying these mutations has become a key diagnostic goal. Companion diagnostics have become an important tool in identifying the mutational cause of cancer and then administering effective therapy for that particular mutation. Furthermore, some tumors acquire new mutations that confer resistance to targeted therapies. Thus, accurate determination of a tumor's mutation status can be a critical factor in determining the appropriateness of particular therapies for a given patient. However, detecting tumor-specific somatic mutations can be difficult, especially if tumor tissue obtained from a biopsy or a resection has few tumor cells in a large background of stromal cells. Tumor-derived mutant DNA can be even more challenging to measure when it is found in very small amounts in blood, sputum, urine, stool, pleural fluid, or other biological samples.

Tumor-derived DNA is released into the bloodstream from dying cancer cells in patients with various types of malignancies. Detection of circulating tumor DNA (ctDNA) has several applications including, but not limited to, detecting presence of a malignancy, informing a prognosis, assessing treatment efficacy, tracking changes in tumor mutation status, and monitoring for disease recurrence or progression. Since unique somatic mutations can be used to distinguish tumor-derived DNA from normal background DNA in plasma, a new class of highly specific DNA-based cancer biomarkers are described with clinical applications that may complement those of conventional serum protein markers. In an embodiment, methods include screening ctDNA for presence of tumor-specific, somatic mutations. In such embodiments, false-positive results are very rare since it would be very unlikely to find cancer-related mutations in the plasma DNA of a healthy individual. Described herein are methods that specifically and sensitively measure rare mutant DNA molecules that are shed into blood from cancer cells. Achieving extremely high detection sensitivity is especially important for detection of a small tumor at an early (and more curable) stage.

Since somatic mutations can occur at many possible locations within various cancer-related genes, a clinically useful test for analyzing ctDNA would need to be able to evaluate mutations in many genes simultaneously, and preferably from many samples simultaneously. In embodiments, analysis of a plurality of mutation-prone regions from a plurality of samples allows more efficient use of large volumes of sequence data that can be obtained using massively parallel sequencing technologies. In an embodiment, labeling molecules arising from a given sample with a sample-specific DNA sequence tag, also known as a barcode or index, facilitates simultaneous analysis of more than one sample. By using distinct barcode sequences to label molecules derived from different samples, it is possible to combine molecules and to carry out massively parallel sequencing on a mixture. Resultant sequences can then be sorted based on barcode identity to determine which sequences were derived from which samples. To minimize chances of misclassification, barcodes are designed so that any given barcode can be reliably distinguished from all other barcodes in the set by having distinct bases at a minimum of two positions.

In most protocols that are currently used to prepare samples for massively parallel sequencing, barcodes are attached after several steps of sample processing (e.g. purification, amplification, end repair, etc.). Barcodes can be attached either by ligation of barcoded sequencing adapters or by incorporation of barcodes within primers that are used to make copies of nucleic acids of interest. Both approaches typically require several processing steps to be performed separately on nucleic acids derived from each sample before barcodes can be attached. Only after barcodes are attached can samples be mixed.

In an embodiment, barcodes are assigned to targeted molecules at a very early step of sample processing. Targeted early barcode attachment not only permits sequencing of multiple samples to be performed in batch, it also enables most antecedent processing steps to be performed in a combined reaction volume. Once barcodes are attached to nucleic acid molecules in a sample-specific manner, molecules can be mixed, and all subsequent steps can be carried out in a single tube. If a large number of samples are analyzed, targeted early barcoding can greatly simplify the workflow. Since all molecules can be processed under identical conditions in a single tube, the molecules would experience uniform experimental conditions, and inter-sample variations would be minimized. In an embodiment, tagging of nucleic acids from different samples can be achieved in consistent proportions and then used to enable quantitative comparisons of nucleic acid concentrations across samples. In addition to quantifying DNA, targeted early barcoding can enable quantifying RNA (e.g., RNA expression levels across different samples). Once barcodes are attached, targeted nucleic acids bearing different sample-specific barcodes can be amplified in a combined reaction volume by competitive end-point PCR, and relative counts of different barcodes in amplified products could be used to quantify associated nucleic acids in various samples. Thus, early barcoding can be used to quantify a total amount of various targeted nucleic acids, and not just variants, across many samples.

In an embodiment, well-defined mixtures of primers are produced containing combinations of sample-specific barcodes and consistent ratios of gene-specific segments. Such primers can be used for targeted early barcoding and subsequent batched sample processing. These primers can also be used for quantitation of DNA or RNA in different samples. In an embodiment, such primers allow parallel processing and analysis of multiple mutation-prone genomic target regions from multiple samples in a simplified and uniform manner.

Embodiments include methods that accurately quantify mutant DNA rather than simply determining its presence or absence. In an embodiment, an amount of mutant DNA provides information about tumor burden and prognosis. Embodiments are capable of analyzing DNA that is highly fragmented due to degradation by blood-borne nucleases as well as due to degradation upon release from cells undergoing apoptotic death. Since somatic mutations can occur at many possible locations within various cancer-related genes, an embodiment can evaluate mutations in many genes simultaneously from a given sample. Embodiments are capable of finding mutations in ctDNA without knowing beforehand which mutations are present in a patient's tumor. An embodiment is able to screen for many different types of cancer by evaluating multiple regions of genomic DNA that are prone to developing tumor-specific somatic mutations. An embodiment includes multiple samples combined together in the same reaction tube to minimize inter-sample variations.

Although the methods described herein have been optimized for measurement of small amounts of mutant circulating tumor DNA (ctDNA) in a background of normal (wild-type) cell-free DNA in the plasma or serum of a patient having cancer, it is understood that they could be applied more broadly to the analysis of nucleic acid variants from a variety of sources. Examples of such sources include, but are not limited to lymph nodes, tumor margins, pleural fluid, urine, stool, serum, bone marrow, peripheral white blood cells, cheek swabs, circulating tumor cells, cerebrospinal fluid, peritoneal fluid, amniotic fluid, cystic fluid, frozen tumor specimens, and tumor specimens that have been formalin-fixed and paraffin-embedded.

### Features

Methods include identifying and measuring low-abundance variants occurring in multiple mutation-prone regions of genomes from multiple samples in parallel. One aspect includes early attachment of sample-specific DNA barcodes to a plurality of nucleic acid targets that are derived from a plurality of samples. Specifically, a mixture of gene-specific primers, all bearing the same barcode, are used to make tagged copies of several different genomic target regions from nucleic acids in a given sample in a single reaction volume. For each additional sample, this process is repeated in a separate reaction volume using a similar mixture of gene-specific primers bearing a different barcode. All members of a given primer mix have the same sample-specific barcode, but different primer mixes have different barcodes. Once barcodes have been attached, the DNA from multiple samples can be combined into a single volume for further processing.

If many DNA targets from many samples are to be analyzed, large numbers of primers would need to be produced, each having different combinations of barcoded 5′ segments and gene-specific 3′ segments. Targeted early barcoding allows combining nucleic acids from different samples and processing of the nucleic acids together in a combined reaction volume. Batched processing has an advantage of simplified workflow and greater experimental consistency and uniformity across different samples. Batched processing decreases potential quantitative variability arising from very small inter-sample concentration or temperature differences. Although the variability may be small at time of initial input, the end result may have substantial variability due to the exponential nature of PCR. Amplification of differently barcoded nucleic acid copies in a combined reaction volume by competitive end-point PCR followed by high throughput sequencing of the products would allow direct enumeration of the various barcodes associated with a given genomic target region. The relative quantity of each targeted nucleic acid in the different samples could be deduced from the relative abundance of the various barcodes within the sequence data.

Another aspect includes producing primers by combining modular oligonucleotide segments. Implementing targeted early barcoding requires generating well-defined mixtures of large numbers of primers. Primer mixtures are produced in such a way that each mixture contains identical proportions of 3′-gene-specific segments, ensuring that target nucleic acids from different samples are copied in consistent ratios. This makes it possible to quantitatively compare nucleic acid concentrations across different samples. In an embodiment, combining modular oligonucleotide segments is used. More specifically, to generate each mixture, a portion of a uniform pool of various gene-specific 3′ oligonucleotide segments is joined to a single, uniquely-barcoded 5′ segment. Since the 3′ segments used to produce each final mixture are derived from a common pool (or master-mix), each uniquely barcoded primer mix has similar proportions of the different 3′ gene-specific segments. Several approaches are described herein for joining the modular 5′ and 3′ segments. This modular approach to producing primer mixes allows the production of thousands of primer and barcode combinations that would have otherwise been very costly and laborious to produce. Furthermore, the consistency of gene-specific primer ratios that can be achieved across different mixes would not be possible by mixing individually synthesized primers. Methods described herein utilize next-generation, high-throughput DNA sequencing technologies to identify and quantify nucleic acid variants. These technologies are able to quickly and inexpensively produce sequences from millions of DNA molecules in a massively parallel fashion. By oversampling sequences of a large number of DNA molecules from a particular genomic region using ultra-deep sequencing, it would be possible to identify and enumerate rare sequence variants. The sensitivity of the sequencing is limited by the inherent error rate of the sequencer since incorrectly read bases might be mistaken for true mutant DNA copies. Mutant ctDNA has been reported to comprise on average 0.2% of total plasma DNA (Diehl et al., Nat Med. 2008; 14: 985-990)-a range in which sequencer misreads can be problematic. This is a limitation of massively parallel sequencing to measure very low-abundance mutations.

Herein methods are described that use clonal overlapping paired-end sequencing to achieve sequence redundancy in mutation-prone regions, thereby allowing base calls to be made with much greater confidence. Embodiments include methods of reducing, suppressing, and distinguishing sequencer-derived errors. Using an Illumina® next-generation sequencing platform, an embodiment includes obtaining a read in one direction from a clonal cluster of DNA molecules, and then subsequently obtaining a read in the opposite direction (from the opposite strand of the duplex). The length of each “paired-end” read can be 36, 50, 75, 100, or 150 bp or longer. An embodiment includes sequencing short PCR amplicons in a paired-end fashion to obtain overlapping reads from both strands of a clone. By designing the mutation-prone region to be in the area of sequence overlap, clonal sequence redundancy can be achieved in this region. Thus, each clonal sequence from a mutation-prone region is read in one direction, and then is proofread in the other direction. Read-pairs that do not have perfect agreement in the overlapping region (after obtaining a reverse-complement of one of the reads) can be attributed to sequencer error, and can be ignored in the final analysis. In this way, sequencer-generated errors in a region of interest can be reduced since a probability of finding the same sequencer error in reads from both strands of a clone is exceedingly low. By reducing the background of sequencer errors, it becomes possible to achieve better detection sensitivity for rare mutant molecules. Detection sensitivity is especially important in patients with early-stage cancers who are likely to have a very low concentration of mutant ctDNA molecules in their blood.

Another aspect includes distinguishing nucleotide misincorporation errors that can be introduced during DNA copying, amplification, or processing. After suppression of sequencer-derived errors, variant sequences are still found that do not correspond to authentic mutations arising from mutant template DNA molecules. A majority of these variant sequences arise from incorporation of incorrect nucleotides when DNA template molecules are copied or amplified. Possible causes of such misincorporation errors include but are not limited to DNA damage (for example, cytosine deamination during heating) or polymerase-induced errors.

To distinguish variant sequences arising from true mutant template molecules versus those arising from misincorporation errors, an embodiment includes molecular lineage tagging. In molecular lineage tagging, a degenerate sequence called a molecular lineage tag (MLT) is incorporated into primers that make a small number of copies (between 2 to 20) of an original template DNA molecule. An MLT is a stretch of degenerate sequence having an approximately equal probability of having A, T, C, or G at each position and can be about 2 to about 10 bases in length, but preferably would be 6, 7, or 8 bases long. An MLT sequence can also be split into segments that are separated by non-degenerate positions within an oligonucleotide.

It is not necessary that each template molecule be tagged with a unique MLT, but only that each template molecule should have a low probability of being tagged with any given MLT-sequence. For example, if the MLT region consisted of 8 degenerate positions, then 4\hat{ }8=65,536 possible MLT sequences could be generated. MLT-containing primers are used to make a limited number of copies of the template DNA molecules, via either a few cycles (2 to 4) of PCR or primer-extension. Thus, each template copy would be tagged with one of 65,536 possible MLT sequences. When these tagged copies are amplified by PCR, the “progeny” molecules derived from amplification of a given “parent” copy should retain the same identifying MLT sequence as the parent molecule. If a variant sequence arose from a true mutant template molecule, then many copies of a given MLT sequence should be associated with that variant sequence (since that MLT was associated with the mutant copy at the beginning of the amplification process). On the other hand, if an error was introduced during amplification or processing, one would expect a smaller number of copies of a given MLT to be associated with the erroneous variant sequence (unless the error occurred at a very early cycle of amplification). It is important to note that if several thousand template molecules are tagged with MLTs, there is a high probability that some MLT sequences may be assigned to more than one template molecule.

With non-unique MLT's, it is less informative to evaluate the percentage of mutant and wild-type sequences associated with a particular MLT sequence. Rather, it is preferable to identify mutant sequences, and then to evaluate distribution of MLT sequences associated with those variants. If the number of sampled clonal sequences (post-amplification) is several-fold greater than the number of tagged template copies, then variant sequences arising from true mutant template molecules would be associated with multiple copies of a given MLT sequence, whereas variants arising from misincorporation errors would be likely to be associated with fewer copies of any given MLT. Analysis of MLT distributions (number of different MLT sequences and number of copies of each sequence) associated with a particular variant made it possible to identify the majority of variants arising from misincorporation errors, thereby further improving the sensitivity for detecting true template-derived mutations.

Another aspect includes distinguishing sequences that are misclassified as belonging to a wrong sample. Such incorrect classification of a sequence can occur if it is associated with an inappropriate barcode. Since barcodes are designed to differ from all other barcodes in a set at a minimum of two distinct positions, misclassification due to barcode sequence errors would be rare. However, cross-over of barcodes has been observed from differently barcoded molecules that undergo combined polymerization or amplification in the same reaction volume. This can happen, for example, if primer-extension stalls before a polymerase has completed extending on a template during a given cycle of PCR. That partially-extended strand (possibly containing a mutant or wild-type sequence) could then anneal to a different template during the next cycle of PCR, and could incorporate an inappropriate barcode. Alternatively, if two strands of DNA containing different barcodes are annealed to each other via a common complementary sequence, the 3′-5′ exonuclease activity of a proofreading polymerase can digest the barcode on one strand and then extend that strand using the opposite strand's barcode as a template. MLT sequences can be used to distinguish sequences derived from such barcode “cross-over” events. If an MLT region is positioned in proximity to or adjacent to a barcode sequence, then it can be used to track the lineage of the barcode. If a variant is tagged with an inappropriate barcode as a result of cross-over during the process of amplification, then one would expect fewer than average copies of a particular MLT sequence to be associated with that barcode/variant combination. To further aid in distinguishing cross-over sequences, a second MLT can be positioned on the opposite side of the mutation-prone region (so that the sequence order, for example, could be MLT-1/Barcode/mutation-prone region/MLT-2). In this case, DNA molecules that undergo cross-over between a barcode and a mutation prone region would also undergo cross-over of MLT-1 between MLT-2. Thus, such crossed-over sequences could be identified because the number of copies of a particular MLT-1/MLT-2 combination would be lower than for sequences that did not undergo cross-over. Thus, MLT sequences can allow differently barcoded molecules to be amplified in a combined reaction volume while maintaining accurate assignment of mutations to specific samples.

Another aspect includes highly-specific tagging, copying, and amplification of several genomic target regions from several samples simultaneously in a single reaction volume while minimizing accumulation of unwanted, spurious amplification products. Such highly multiplexed processing and amplification is prone to accumulation of spurious products because of the presence of large numbers of different primers. Having a complex mixture of primers with different combinations of barcodes, degenerate sequence regions, and gene-specific regions in a single PCR amplification can lead to formation of many primer dimers and non-specific amplification products. An embodiment includes multi-step tagging and amplifying without having to compromise primer concentrations. An embodiment of a process includes highly stringent purification of desired amplification products between each amplification step to remove unextended primers, spurious extension products, and genomic template DNA as well as enzyme, buffer, and nucleotides. An embodiment utilizes biotin-tagged oligonucleotides to mediate specific isolation of desired products. Another embodiment utilizes high-temperature washes when using biotin-tagged oligonucleotides. Another embodiment includes digesting unwanted single-stranded products and primers with an exonuclease to further improve amplification specificity. An embodiment also uses nested primers to provide further selectivity for desired products. An embodiment includes universal PCR primers for the final amplification. Under the stringent conditions described herein, universal PCR primers can be used for the final amplification without significant accumulation of spurious products.

### Methods

### Producing Combinations of Modular Oligonucleotide Segments for Tagging of Nucleic Acids

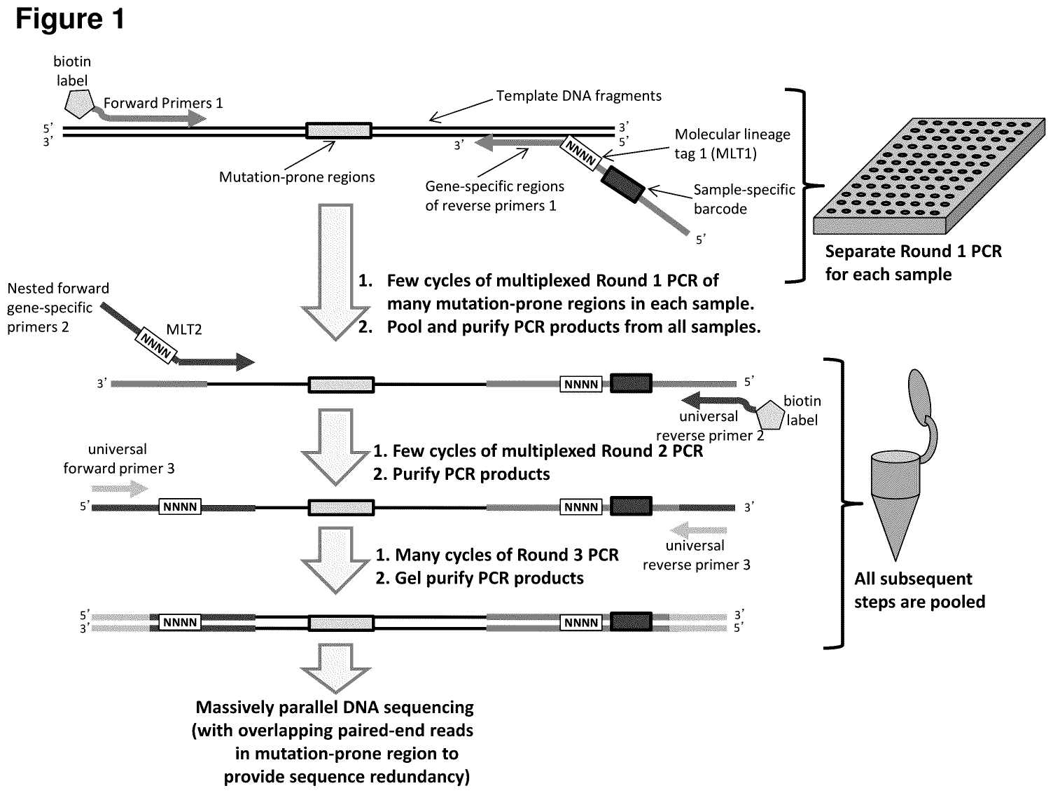

In an embodiment, tagged copies of multiple nucleic acid targets are made from template DNA or RNA derived from a given sample. To produce such tagged copies, a mixture of primers is used in which the 3′-segments of the primers are able to hybridize to RNA or DNA targets by sequence complementarity (as illustrated, for example, by the reverse primers 1 in FIG. 1). A polymerase (such as a reverse transcriptase or a DNA polymerase) can then be used to extend the primers in the 5′ to 3′ direction using the targeted nucleic acids as templates. In an embodiment, a sample-specific DNA barcode sequence can be incorporated into the 5′-segment of each primer such that the barcode becomes attached to the copy of the target template after undergoing primer-extension. Since multiple target templates are to be copied from a given sample in a single reaction tube, a mixture of primers is required having various target-specific sequences in their 3′-segments and all having the same sample-specific barcode sequence in their 5′-segments. If several different samples are to be analyzed, then similar mixtures of primers must be made for each sample, with each mixture containing a unique, sample-specific barcode sequence in the 5′-segment. In some embodiments, the 5′-segment of each primer can also contain other elements such as sequencing adapters (to facilitate sequencing of the copied DNA), binding sites for PCR primers, or stretches of degenerate sequence (having equal probability of A, C, T, or G bases at each position) that can serve as tags to follow the lineage of molecules during copying, amplification, and sequencing.

In an embodiment, a barcode comprises a unique sequence (typically 6 to 12 nucleotides long) that is used to identify molecules derived from a particular sample after molecules from multiple samples are pooled and sequenced in batch. In an embodiment, a computer program can be used to sort clonal sequences derived from each molecule based on barcode identity. In order to minimize the chance that a sequence derived from one sample might be misclassified as being derived from another sample, each barcode sequence is designed to differ from all other barcodes in the set by at least 2 nucleotides (so that a single sequencing error would not lead to misclassification).

In an embodiment, multiple gene-specific primer regions (at the 3′-ends of primers) are attached in separate batches, to unique sample-specific barcodes (near the 5′-regions of primers). If many genomic targets are to be analyzed from many samples, the number of combinations of primer 3′-ends and 5′-ends can become very large. For example, if 40 target gene regions are to be evaluated from 96 different samples, 40×96=3,840 different oligonucleotides would need to be made, each with a unique combination of 3′ gene-specific sequence and 5′ barcode. If conventional oligonucleotides were individually synthesized, a mixture of 40 different gene-specific primers having a particular barcode would be used to primer-extend nucleic acid targets from a given sample within a single tube. Thus, all 40 target regions would be tagged with the same sample-specific barcode. However, synthesis and purification of 3,840 oligonucleotides individually would be impractical. Because termination sequences would be abundant when making long primers, full-length oligonucleotides would have to be purified by methods including but not limited to polyacrylamide gel electrophoresis, high performance liquid chromatography, or reverse-phase cartridge purification.

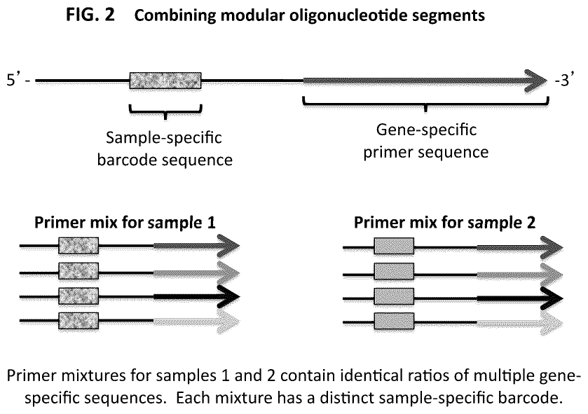

To address the need for producing uniform mixtures of multiple gene-specific primer, with each mixture having a unique barcode sequence, embodiments are described in which combinations of modular gene-specific 3′ oligonucleotide segments can be attached to a modular barcoded 5′ oligonucleotide segment. In various embodiments, production of modular oligonucleotides allows multiple gene-specific 3′ segments to be synthesized and uniformly mixed, and then attached in separate batches to each barcoded 5′ segment (FIG. 2). Resulting uniquely barcoded primer mixes would each have consistent ratios of gene-specific primer sequences. In some embodiments, such uniform primer mixes could be used to copy and label DNA or RNA molecules from different samples with consistent efficiency (so that the resulting tagged copies would be proportionate to the amount of target nucleic acids in each sample). Subsequent pooled amplification of differently barcoded molecules by competitive PCR, followed sequencing to count barcoded sequences, would enable accurate quantitation of DNA or RNA targets in the various samples. Such uniform primer mixes would be very difficult to achieve by simply mixing individually synthesized primers.

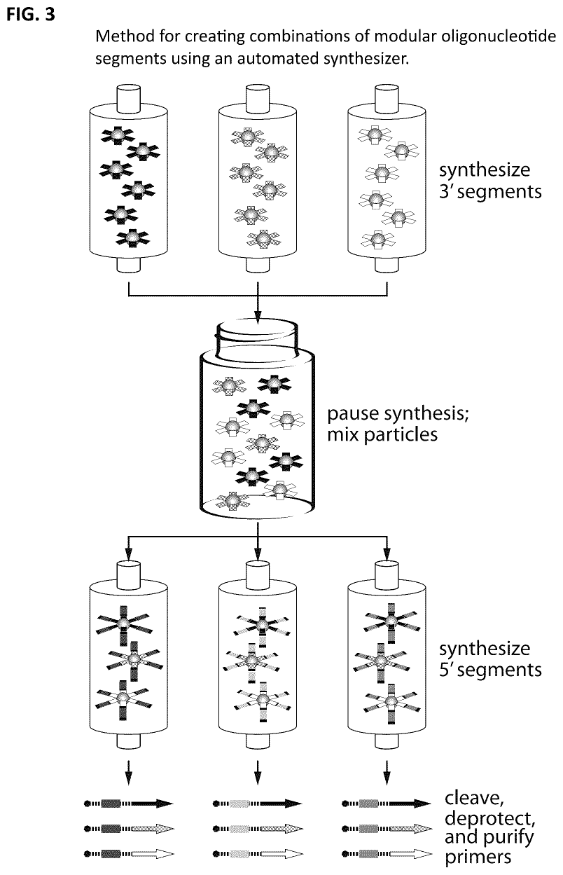

In some embodiments, multiple 3′ oligonucleotide segments are produced and mixed, and then the mixture is joined in separate batches to unique 5′ oligonucleotide segments. In an embodiment (FIG. 3), different 3′-segments can be synthesized in separate columns on an automated oligonucleotide synthesizer (on solid supports). Synthesis can then be paused and the solid supports from the different columns can be uniformly mixed. Then the mixture can be dispensed into several fresh columns. Synthesis can then be continued, adding a uniquely barcoded 5′ segment to each new column. After cleavage, deprotection, and purification, the desired uniquely barcoded uniform primer mixtures are obtained. In another embodiment, mixtures of 5′-phosphorylated 3′-segments can be ligated to different barcoded oligonucleotides using splint-mediated enzymatic ligation. In another embodiment, primer extension can be used to produce combinations of modular segments. In this approach, a single barcoded 5′ oligonucleotide can be hybridized via a common sequence to a mixture of complementary templates. These templates can be designed to produce various gene-specific 3′-ends when the barcoded oligonucleotide is primer extended on these templates. In an embodiment, biotin tags on the templates can be used to separate the templates from the desired uniquely barcoded primer mix. Similar primer extension reactions can be performed in separate reaction volumes using different barcoded oligonucleotides to produce several similar uniform primer mixes. In yet another embodiment, mixtures of 3′ oligonucleotide segments having reactive chemical conjugation moieties on their 5′-end can be combined in separate batches with uniquely barcoded 5′ oligonucleotides segments having reactive conjugation moieties on at their 3′ ends. Such chemical conjugation would allow post-synthesis combination of oligonucleotide segments. Special conjugation chemistries have been previously described that can conjugate two oligonucleotide segments together leaving a phosphodiester bond at the junction (or similar bond that would be compatible with subsequent enzymatic processes).

### Isolation of Template DNA

Embodiments provide methods for purification or isolation of DNA or RNA from various clinical or experimental specimens. Many kits and reagents are commercially available to facilitate nucleic acid purification. Depending on the type of sample to be analyzed, appropriate nucleic acid isolation techniques can be selected. Substances that might inhibit subsequent enzymatic reaction steps (such as polymerization) should be removed or reduced to non-inhibitory concentrations in purified DNA or RNA samples. A yield of nucleic should be maximized whenever possible. It would be disadvantageous to lose DNA during purification, wherein the lost DNA might include rare variant DNA. When isolating DNA from plasma, about 10 ng to 100 ng of cell-free DNA can be purified from 1 mL of plasma, which corresponds to 3,500 to 35,000 genome copies. To note, DNA yields can vary dramatically, especially in patients with an ongoing disease process such as cancer.

In an embodiment, DNA can also be analyzed from other sample types, including but not limited to the following: pleural fluid, urine, stool, serum, bone marrow, peripheral white blood cells, circulating tumor cells, cerebrospinal fluid, peritoneal fluid, amniotic fluid, cystic fluid, lymph nodes, frozen tumor specimens, and tumor specimens that have been formalin-fixed and paraffin-embedded.

### Producing a Limited Number of Tagged Copies of Targeted Nucleic Acids

In an embodiment, a limited number of tagged copies (e.g., fewer than 20) of targeted nucleic acid molecules are made at an early step in the process. After DNA or RNA is purified from the original sample, targeted nucleic acid template molecules can be copied by specifically hybridizing and polymerizing tagged primers. When a plurality of target regions are to be copied and tagged from a given sample, a mixture of modular barcoded primers can be used (as described above). In an embodiment, targeted nucleic acid regions are mutation-prone regions (also called mutation hotspots). A mixture of primers for a given sample can contain sequences at their 3′-ends that specifically hybridize to an area of DNA near or adjacent to a target region. All primers used for a given sample would have the same sample-specific barcode sequence, and different samples would have different barcodes. In some embodiments, the primers can also contain stretches of degenerate bases known as molecular lineage tags (MLTs) that can be helpful in distinguishing sequences arising from true mutant template molecules versus those arising from misincorporation errors occurring during amplification or processing. The MLTs can also help to identify sequences that are assigned to the wrong barcode due to cross-over of barcodes during pooled amplification of differently barcoded molecules. In an embodiment, primers can also contain adapter sequences that are necessary for sequencing, and universal primer binding sites that can be used in subsequent amplifications.

In an embodiment, a DNA polymerase can be used to extend the primers on hybridized templates, thus producing copies of the target nucleic acids with sample-specific barcodes attached. A DNA polymerase can be a thermostable or non-thermostable enzyme, and may or may not have proofreading activity. Examples of polymerases include, but are not limited to, Taq, Phusion®, VentR®, Pfu, Pfx, DNA Polymerase I (Klenow fragment), or reverse transcriptase. When specific primer annealing and extension is to be carried out at temperatures above 50° C., thermostable polymerases with hot-start capability are preferred in order to minimize spurious polymerization at room temperature during reaction set-up. Copies of template nucleic acids can be made by a single primer extension step, by a few cycles of primer extension (1 to 10 cycles, with heat-denaturation of the extended products between cycles), or by a few cycles of PCR in which opposite primers are also added (2 to 5 cycles). A few tagged copies of each template molecule can be produced so that a complete sampling of sequences can be obtained even if there is some loss of copies during the various purification, processing, and amplification steps. However, the number of tagged copies must be limited to avoid assigning too many different MLTs to each template molecule, which would require greater sequencing depth for analysis. In an embodiment, after a limited number of tagged copies are made, the polymerase is inactivated, and barcoded copies from different samples can then be pooled into a combined volume for further processing.

### Purification of Tagged Copies

In an embodiment, tagged, primer-extended copies of target sequences are purified away from un-extended and non-specifically extended primers and from excess template nucleic acids. Purification also removes other reaction components such as buffer, dNTPs, and polymerase. Removal of un-extended primers and non-specifically extended primers is preferred so that they are not carried over to the next polymerization step. Also, removal of excess primers and template molecules allows greater specificity of polymerization in subsequent steps.

In an embodiment, purification of specifically tagged and extended products is mediated by capture using biotin-labeled complementary oligonucleotides that hybridize to the specifically extended products. Oligonucleotides can be designed to anneal to sequences produced when tagged primers are extended beyond the mutation-prone region (or target region). Such hybridization of the biotin-labeled capture oligonucleotides to the extended tagged copies can be achieved either by using the biotinylated primers in PCR (FIG. 1), or by subsequently annealing them to primer-extended copies (FIG. 10). In an embodiment, immobilized streptavidin (or an analogue with affinity for biotin) is used to isolate and purify the tagged, extended copies that hybridize to the capture oligonucleotides. Immobilized streptavidin is available in many forms, including but not limited to surface-bound, agarose bead-bound, magnetic bead-bound, or filter-bound. In some embodiments, removal of non-specifically annealed nucleic acids can be achieved by washing the bound molecules at room temperature or at elevated temperatures that would selectively disrupt short, non-specific stretches of hybridization, but would not disrupt specifically-annealed products. In some embodiments, nuclease treatment of the bound molecules can also be used to digest non-specifically annealed products. Nucleases that could be used include but are not limited to Exonuclease I, Exonuclease VII, and Rec Jf. In some embodiments, elution of specifically-annealed copies can be achieved by heat-denaturation or by alkaline-denaturation to separate biotin-labeled strands from the desired single-stranded tagged copies. Biotin labeled strands should remain attached to the immobilized streptavidin since the biotin-streptavidin interaction is not substantially disrupted by heat or moderate alkaline conditions.

In another embodiment, specifically primer-extended copies can be purified by carrying out limited cycles of PCR and then digesting single-stranded nucleic acids to remove un-extended primers. In yet another embodiment, oligonucleotides can be specifically hybridized to primer-extended products to protect their 3′-ends from digestion by a 3′ to 5′ single-stranded exonuclease such as Exonuclease I.

Double-stranded products that survive digestion can be purified by a variety of approaches, including but not limited to ethanol precipitation, silica membrane partitioning, or binding to magnetic Solid Phase Reversible Immobilization (SPRI) beads.

### Second Round of Tagging, Copying, and Purification

In an embodiment, the tagged, pooled, and purified DNA copies from multiple samples can be subjected to another round of limited-cycle primer-extension or limited-cycle PCR (similar number of cycles as described for the first round). Primers used in this second round would be designed to incorporate MLTs on the opposite side of the mutation-prone region relative to the MLTs incorporated in the first round (FIG. 1). This second MLT region could be used to distinguish sequences arising from barcode cross-over events that occurred during pooled amplification or processing. Use of nested primers in the second round of PCR or primer-extension would provide additional selectivity for the targeted genomic sequences. In an embodiment, primers used in the second round could contain universal primer binding sites that would be used for subsequent amplification with universal primers. In an embodiment, primers could also contain adapter sequences that facilitate sequencing using a next-generation sequencer.

In an embodiment, a limited number of specifically primer-extended copies produced in the second round could be purified away from un-extended or non-specifically extended primers and other reaction components using similar approaches as described for the first round. In an embodiment, purification can be achieved using biotinylated capture oligonucleotides designed to specifically hybridize to sites on the opposite primer-extended strands (relative to the hybridization sites of the biotinylated oligonucleotides used in the first round). In an embodiment, nuclease treatment may be used to digest un-extended or non-specifically extended primers.

### Amplification and Purification of Specifically Copied and Tagged Products

In an embodiment, products from the first two rounds of copying, tagging, and purification are used as templates for further PCR amplification. In an embodiment, universal primers are used for PCR that are designed to bind to sequences introduced by primers in the first two rounds. Since universal primers are used, it is very important that only desired targeted products remain as templates for the final PCR after the second-round purification. Presence of even small amounts of primer dimers or other spurious products could lead to competitive amplification of undesired templates by the universal primers. In an embodiment, this round of PCR can be carried out for a larger number of cycles than were used in the first 2 rounds. A total of 5 to 40 PCR cycles may be used, depending on the amount of template nucleic acid present and the number of samples being multiplexed. A final PCR is designed to produce sufficient DNA as required for massively parallel sequencing (which can differ depending on the sequencing platform being used). In some embodiments, a final PCR may not be necessary if the required input of the sequencer is satisfied by the amount of DNA product generated after the first 2 rounds. In some embodiments, the DNA products are gel-purified to select products of the desired size and to eliminate unused primers before subjecting to massively parallel sequencing. In some embodiments, other approaches to purification could be used, including but not limited to high-performance liquid chromatography, capillary electrophoresis, silica membrane partitioning, or binding to magnetic Solid Phase Reversible Immobilization (SPRI) beads.

### Massively Parallel Sequencing and Data Analysis

In an embodiment, a next-generation sequencer is used to obtain large numbers of sequences from the tagged, amplified, and purified products. Clonal sequences (each sequence arising from a single nucleic acid molecule) produced by such a sequencer can be used to identify and quantify variant molecules using an approach known as ultra-deep sequencing. In principle, because large numbers of sequences can be obtained for each target site and for each sample, rare variants can be detected and measured. However, the error rate of the sequencer can limit the sensitivity of detection because such errors might be mistaken as true variants. To minimize the contribution of sequencer errors, an embodiment uses clonal overlapping paired-end sequences. By separately sequencing opposite strands of DNA from each clonal population, and comparing the overlapping regions of the sequences, the vast majority of variants arising from sequencer errors can be eliminated. In an embodiment, the region of sequence overlap is designed to be in the mutation-prone area. In an embodiment, only read-pairs that perfectly match in the overlapping region are retained for further analysis. For such analysis, instruments that produce clonal paired-end reads (such as the Illumina platform) are preferred. In some embodiments, other massively parallel sequencing platforms that provide sequence redundancy can also be utilized.

In an embodiment, errors introduced into the DNA during amplification or processing can be distinguished from true template-derived mutant sequences by analyzing the distribution of molecular lineage tags (MLTs) associated with variant sequences. In an embodiment, MLTs can also be used to distinguish sequences bearing incorrect barcodes due to cross-over events during pooled amplification.

The present technology may be better understood by reference to the following examples. These examples are intended to be representative of specific embodiments of the invention and are not intended to limit the scope of the invention.

### EXAMPLES

### Example 1

This example demonstrates application of a deep sequencing approach in which 3 mutation hotspot regions were analyzed from multiple plasma samples. The method in this example includes redundancy within each clonal sequence to produce extremely high quality base-calls in short, mutation-prone regions of plasma DNA. Amplification of both mutated and wild-type sequences was carried out by unbiased PCR in the same tube, ensuring highly accurate and reproducible quantitation. The scheme was designed to have flexibility to simultaneously analyze mutations in several genes from multiple patient samples, making it practically feasible to screen plasma samples for mutant ctDNA without prior knowledge of the tumor's mutation profile.

### Materials and Methods

### Patient Plasma and Tumor Samples

Under the approval of the Human Investigation Committees at the Yale School of Medicine and at Lawrence & Memorial Hospital, plasma samples were obtained from 30 patients with stage I-IV non-small cell lung cancer (NSCLC) between July 2009 and July 2010. Informed consent was obtained from all patients. Most patients were recruited in the radiation oncology clinic, and underwent treatment with radiation therapy, chemotherapy, targeted systemic therapy, and/or surgery. Whenever possible, blood samples were collected from patients before starting the current course of treatment and then at subsequent times during and after treatment. A total of 117samples were obtained. Formalin-fixed, paraffin-embedded tumor specimens were obtained for all patients with non-squamous histology whose tumors had not already been tested for mutations by a clinical laboratory, and for whom sufficient tissue was available in the block after standard pathology evaluation.

### Extraction and Amplification of Plasma DNA

Blood was collected in EDTA-containing tubes (Becton Dickinson) and was centrifuged at 1000 g for 10 minutes within 3 hours of collection. Plasma was transferred to cryovials, being careful to avoid the buffy coat, and was stored at −80° C. until further processing. Frozen plasma aliquots stored at −80° C. were thawed to room temperature, and DNA was purified using the QIAamp® DNA Blood Mini kit (Qiagen Sciences, Valencia, CA) as per the manufacturer's instructions. 5 μg of carrier RNA was added to each 200 μL plasma sample as recommended to improve adsorption of low-concentration nucleic acids to the silica membrane.

Purified plasma DNA was then subjected to 2 rounds of amplification by PCR (in triplicate) using primers designed to amplify short DNA segments that included codons 12 and 13 of KRAS, codon 858 of EGFR, and codon 600 of BRAF. The sequences of the primers used in both rounds of PCR are listed in Table 1.

TABLE 1
Primers used in first and second rounds of PCR.

| PCR | | SEQ ID |
|---|---|---|
| Primer | Sequence (5′-3′) | NO: |
| Round 1 | GGCCTGCTGAAAATGACTGAATATAAAC | 1 |
| Forward | | |
| KRAS* | | |
| Round 1 | TTCGTCCACAAAATGATTCTGAATTAGC | 2 |
| Reverse | | |
| KRAS* | | |
| Round 1 | TCATGAAGACCTCACAGTAAAAATAGGTG | 3 |
| Forward | | |
| BRAF* | | |
| Round 1 | CACAAAATGGATCCAGACAACTGTTC | 4 |
| Reverse | | |
| BRAF* | | |
| Round 1 | GTACTGGTGAAAACACCGCAGCAT | 5 |
| Forward | | |
| EGFR* | | |
| Round 1 | CTTACTTTGCCTCCTTCTGCATGGTATT | 6 |
| Reverse | | |
| EGFR* | | |
| Round 2 | GG[FWD | 7 |
| Forward | BC]CGAACAGTCTCCGAATATAAACTTGTGG | |
| KRAS | TAGTTGG | |
| Round 2 | GC[REV | 8 |
| Reverse | BC]GGATGAGTGCAGTGAATTAGCTGTATCG | |
| KRAS | TCAAG | |
| Round 2 | GG[FWD | 9 |
| Forward | BC]CGAACAGTCTCCAAATAGGTGATTTT | |
| BRAF | GGTCTAGC | |
| Round 2 | GC[REV | 10 |
| Reverse | BC]GGATGAGTGCAGCCAGACAACTGTTCAA | |
| BRAF | ACTGA | |
| Round 2 | GG[FWD | 11 |
| Forward | BC]CGAACAGTCTCCCAGCATGTCAAGAT | |
| EGFR | CACAGATT | |
| Round 2 | GC[REV | 12 |
| Reverse | BC]GGATGAGTGCAGGCATGGTATTCTTT | |
| EGFR | CTCTTCC | |

*Primers were gel-purified prior to use.
FWD BC = Forward barcode
REV BC = Reverse barcode

In a first round of PCR, all hotspot regions from a given sample were amplified in a multiplexed fashion. Three aliquots of purified plasma DNA from each sample were used as templates in three identical multiplexed PCRs containing 1× Kapa Fidelity buffer (Kapa Biosystems, Inc., Woburn, MA), 300 μM each dNTP, 50 nM each primer (Round 1 Forward and Reverse KRAS, BRAF, and EGFR primers), and 1 unit/50 μL HiFi Hotstart DNA polymerase (Kapa Biosystems). Mineral oil was added to all PCR tubes to minimize evaporation during heating. Temperature cycling parameters were 95° C. for 2 minutes, followed by 35 cycles of 98° C. for 20 sec, 64° C. for 20 sec, and 72° C. for 30 sec. A final extension was performed at 72° C. for 1minute, prior to cooling the reaction at 4° C. EDTA was then added at a final concentration of 5mM to stop polymerase activity.

The amplicons from each first round PCR were diluted 5000-fold and used as templates for 3 separate second round PCRs to individually amplify the hotspot regions of KRAS, BRAF, or EGFR. To promote specific amplification, the second-round primers were nested relative to the primers used in the first round of PCR. The nested primers were labeled with sample-specific barcode sequences to allow multiplexed sequencing of DNA from many samples. The barcode sequences were 6 nucleotides in length, and were designed to differ from all other barcodes in the set at a minimum of 2 positions so that a single sequencing error would not lead to misclassification of samples. Different combinations of 16 forward and 16 reverse barcoded primers could be used to uniquely identify up to 256 different samples. PCR was carried out using the same reaction conditions as were used in the first round, with the following modifications: the annealing temperature was increased to 65° C., and the 3 pairs of multiplexed primers were replaced with a single pair of barcoded primers (Round 2 Forward and Reverse KRAS, BRAF, or EGFR primers listed in Table 1) at a final concentration of 200 nM each. After addition of 5 mM EDTA, the PCR products were mixed together to produce 3 pools, one for each of the 3 replicate reactions. All PCR steps were carried out using a high-fidelity polymerase (HiFi HotStart, Kapa Biosystems).

### Production of Barcoded PCR Primers

In order to build flexibility and scalability into the design of the deep sequencing scheme, barcoded oligonucleotides and gene-specific PCR primers were combined in a modular fashion, as illustrated in FIG. 9. A set of 16 unique barcodes was produced for the forward primers, and a different set of 16 barcodes was produced for the reverse primers. These barcodes were 6nucleotides in length, and were designed to differ from all other barcodes in the set by at least 2nucleotides (to minimize the probability that miscalled bases would cause misclassification of sequences). Each barcode was incorporated into an oligonucleotide, which was primer-extended using a partially complementary single-stranded template containing the reverse-complement of the gene-specific primer sequence. The sequences of the template and barcode-containing oligonucleotides are listed in Table 2.

TABLE 2
Oligonucleotides used to produce barcoded primers.

| Oligonucleotide | Sequence (5′-3′) | SEQ ID NO: |
|---|---|---|
| Forward KRAS | Biotin- | 13 |
| template | CCAACTACCACAAGTTTATATTCGGAGACTGTTCG | |
| Forward EGFR | Biotin-AATCTGTGATCTTGACATGCTGGGAGACT | 14 |
| template | GTTCG | |
| Forward BRAF | Biotin-GCTAGACCAAAATCACCTATTTGGAGAC | 15 |
| template | TGTTCG | |
| Forward barcode | GGAACCTTCGAACAGTCTCC | 16 |
| 1 oligo | | |
| Forward barcode | GGAACGTACGAACAGTCTCC | 17 |
| 2 oligo | | |
| Forward barcode | GGAAGCATCGAACAGTCTCC | 18 |
| 3 oligo | | |
| Forward barcode | GGAAGGAACGAACAGTCTCC | 19 |
| 4 oligo | | |
| Forward barcode | GGATCCATCGAACAGTCTCC | 20 |
| 5 oligo | | |
| Forward barcode | GGATCGAACGAACAGTCTCC | 21 |
| 6 oligo | | |
| Forward barcode | GGATGCAACGAACAGTCTCC | 22 |
| 7 oligo | | |
| Forward barcode | GGATGGTACGAACAGTCTCC | 23 |
| 8 oligo | | |
| Forward barcode | GGTACCTACGAACAGTCTCC | 24 |
| 9 oligo | | |
| Forward barcode | GGTACGAACGAACAGTCTCC | 25 |
| 10 oligo | | |
| Forward barcode | GGTACGTTCGAACAGTCTCC | 26 |
| 11 oligo | | |
| Forward barcode | GGTAGCTTCGAACAGTCTCC | 27 |
| 12 oligo | | |
| Forward barcode | GGTAGGATCGAACAGTCTCC | 28 |
| 13 oligo | | |
| Forward barcode | GGTTCGATCGAACAGTCTCC | 29 |
| 14 oligo | | |
| Forward barcode | GGTTGCATCGAACAGTCTCC | 30 |
| 15 oligo | | |
| Forward barcode | GGTTGCTACGAACAGTCTCC | 31 |
| 16 oligo | | |
| Reverse KRAS | Biotin- | 32 |
| template | CTTGACGATACAGCTAATTCACTGCACTCATCC | |
| Reverse EGFR | Biotin- | 33 |
| template | GGAAGAGAAAGAATACCATGCCTGCACTCATCC | |
| Reverse BRAF | Biotin- | 34 |
| template | TCAGTTTGAACAGTTGTCTGGCTGCACTCATCC | |
| Reverse barcode 1 | GCAATCAAGGATGAGTGCAG | 35 |
| oligo | | |
| Reverse barcode 2 | GCAATGATGGATGAGTGCAG | 36 |
| oligo | | |
| Reverse barcode 3 | GCAAGATAGGATGAGTGCAG | 37 |
| oligo | | |
| Reverse barcode 4 | GCAACATTGGATGAGTGCAG | 38 |
| oligo | | |
| Reverse barcode 5 | GCATCATAGGATGAGTGCAG | 39 |
| oligo | | |
| Reverse barcode 6 | GCATAGTTGGATGAGTGCAG | 40 |
| oligo | | |
| Reverse barcode 7 | GCATCAATGGATGAGTGCAG | 41 |
| oligo | | |
| Reverse barcode 8 | GCATGTATGGATGAGTGCAG | 42 |
| oligo | | |
| Reverse barcode 9 | GCTAACATGGATGAGTGCAG | 43 |
| oligo | | |
| Reverse barcode | GCTAGTAAGGATGAGTGCAG | 44 |
| 10 oligo | | |
| Reverse barcode | GCTATGTAGGATGAGTGCAG | 45 |
| 11 oligo | | |
| Reverse barcode | GCTTACAAGGATGAGTGCAG | 46 |
| 12 oligo | | |
| Reverse barcode | GCTTCATTGGATGAGTGCAG | 47 |
| 13 oligo | | |
| Reverse barcode | GCTTAGTAGGATGAGTGCAG | 48 |
| 14 oligo | | |
| Reverse barcode | GCTTCTAAGGATGAGTGCAG | 49 |
| 15 oligo | | |
| Reverse barcode | GCTACAATGGATGAGTGCAG | 50 |
| 16 oligo | | |

Barcode sequences are boldfaced and underlined.

Each forward barcode oligo (8 μM) was annealed to each forward template oligo (8 μM) in separate reaction tubes containing 1× NEBuffer 2 (New England Biolabs, Ipswich, MA), 200μM each dNTP, and 1 mM dithiothreitol. Annealing was carried out by heating the solution to 95° C. for 2 minutes, 60° C. for 1 minute, and then slowly cooling to 25° C. over approximately 15minutes. All possible combinations of forward barcode and template oligos were produced. The set of reverse oligos were annealed in a similar manner. 1 unit/10 μL of DNA polymerase I, Large (Klenow) Fragment (New England Biolabs) was added to each tube, and the reaction was incubated at 25° C. for 30 minutes. The reaction was stopped by adding 25 mM ethylenediaminetetraacetic acid (EDTA) and heating to 75° C. for 20 minutes. A biotin tag attached to the 5′-end of the template oligonucleotide was used to purify the primer-extended products from the reaction mix by binding to high capacity streptavidin-coated agarose resin (ThermoFisher Scientific, Wilmington, MA) (5 μL resin slurry added per 50 μL reaction). The resin particles were agitated constantly in the solution at room temperature for 8 hours. The resin was washed three times in buffer containing 10 mM Tris pH 7.6 and 50 mM NaCl. The barcoded PCR primers were then released from the resin-bound template oligos into a fresh 40 μL volume of the same buffer by heat denaturation at 95° C. for 1 minute. After concentration adjustment, the primers were ready for use in PCR.

### Analysis of Cell Line DNA

Genomic DNA was purified from human cancer cell lines using the same method used for purifying plasma DNA, after suspending cells in 0.2 mL of phosphate-buffered saline. The following cell lines were used: A549 (having a KRAS Glyl2Ser mutation), H1957 (having an EGFR Leu858Arg mutation), and YUSAC (having a BRAF Val600Glu mutation). Cells were passed in culture for no more than 6 months after being thawed from original stocks. Because cell lines were used only for analysis of short regions of genomic DNA, authentication of lines by our laboratory was limited to sequencing of those regions. To test the performance of the deep sequencing method for a particular gene, DNA derived from cells known to be either mutant or wild-type with respect to that gene was mixed in various ratios between 10,000:1 and 1:10,000. Cell line DNA samples were then amplified and sequenced according to the same methods that were used for plasma DNA.

### Ultra-Deep Sequencing

Barcoded PCR products from all samples were mixed to produce 3 separate pools, each corresponding to one set of replicate reactions. Uniquely indexed TruSeq® adapters (Illumina, Inc., San Diego, CA) were ligated to each of the 3 pools of PCR amplicons using a modified version of the manufacturer's protocol. Amplicon pools were purified by phenol-chloroform-isoamyl alcohol (PCA, Sigma-Aldrich Co., St. Louis, MO) extraction followed by ethanol precipitation. Addition of deoxyadenosine to the 3′-ends of the blunt-ended amplicons was performed according to Illumina's recommendations. PCA extraction and ethanol precipitation were again used for purification. TruSeq adapters were ligated and the products were purified on a 2% agarose gel according to the standard protocol. DNA concentration was estimated using a Bioanalyzer 2100 (Agilent Technologies, Santa Clara, CA). Without further amplification, the 3 pools were combined and loaded onto a single lane of an Illumina HiSeq® 2000 instrument. Prior to loading, the samples were diluted by adding between 2-and 8-fold excess Phi-X DNA to improve cluster discrimination. Sequencing was carried out in multiplexed, 75 base pair, paired-end mode at the Yale Center for Genomic Analysis.

### Data Analysis

A computer script was written to filter, assort, align, and count millions of paired-end sequences. First, a read-pair was assigned to a data bin based on the barcode of each read in the pair. Then, based on PCR primer sequences, the pair was assigned to one of the reference genes. Next, the longest stretch of perfect sequence agreement between each pair of reads was determined, and this was used to align the reads to the reference sequence for the gene. A read pair was discarded if either member did not pass Illumina filtering or a nucleotide was reported to be “.”; if there was an inconsistency in barcodes, strands, or PCR tags; or if their region of perfect sequence agreement was less than 36 nucleotides in length. Finally, variant sequences confirmed by reads from both strands were identified and counted within each data bin based on comparison to the reference sequence. A module used to perform sequence alignments using a Smith-Waterman algorithm was taken, with permission, from Dr. Conrad Huang, Resource for Biocomputing, Visualization & Informatics, University of California, San Francisco. A module used to determine the longest common substring was taken from a web resource.

### Confirmation of Mutations in Tumor Tissue

Genomic DNA was isolated from paraffin-embedded tumor tissue samples using the QuickExtract™ FFPE DNA Extraction Kit (Epicentre Biotechnologies, Madison, WI). Mutation hotspot regions of KRAS, BRAF, and EGFR were amplified using the same PCR primers that were used in the first round of PCR described above. Sanger sequencing was performed on gel-purified amplicons, and mutations were identified from chromatograms using Mutation Surveyor software (SoftGenetics LLC, State College, PA).

### Determining the Absolute Concentration of Mutant DNA in Plasma

Real-time quantitative PCR was used to measure the concentration of KRAS DNA fragments in each patient's plasma sample. This value was multiplied by the fraction of mutant molecules as determined by deep sequencing in order to calculate the absolute mutant KRAS DNA concentration. PCR conditions were the same as those used in the first round of amplification described above except for the use of a single pair of primers (Round 1 KRAS Fwd and Rev) at 200 nM final concentration, and the addition of SYBR® Green dye (Stratagene, La Jolla, CA) at 1:60,000 final dilution. Amplification was carried out using an IQ5 Real-time PCR Detection System with version 2.1 software (Bio-Rad Laboratories, Hercules, CA). To enable determination of absolute copy numbers, a standard curve was generated using known concentrations of a cartridge-purified oligonucleotide that was designed to mimic the fragment of KRAS DNA being amplified from plasma. The sequence of the oligonucleotide was: 5′-AAGGCCTGCTGAAAATGACTGAATATAAACTTGTGGTAGATGGAGCTGGTGGCGTA AGCAAGAGTG CCTTGACGATACAGCTAATTCAGAATCATTTTGTGGACGAATA-3′ (SEQ ID No: 51). Real-time PCRs were performed in triplicate, and the KRAS DNA concentration was determined using the mean of the 3 measurements.

### Results

### Error Suppression Reveals Low-Abundance Variants

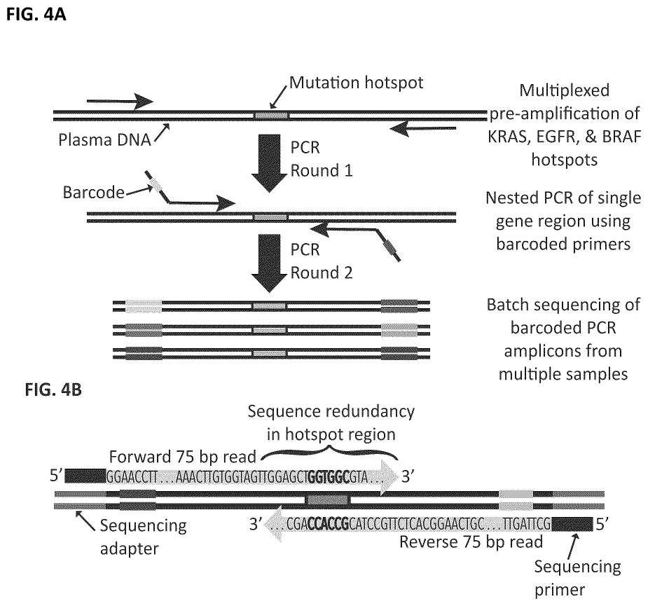

To determine the relative abundance of tumor-specific mutations, massively parallel sequencing was performed on PCR amplicons derived from plasma DNA fragments containing known mutation hotspots. Thousands of clonal sequence reads from each plasma sample were compared to reference sequences in order to identify and quantify variants. For proof of concept, analysis was restricted to frequently mutated codons within 3 oncogenes that commonly develop somatic mutations in various malignancies: codons 12 and 13 of KRAS, codon 600 of BRAF, and codon 858 of EGFR. By designing PCR primers that flank very short regions (<50 bp) surrounding these mutation hotspots, adequate amplification of highly fragmented plasma DNA could be ensured and greater sequence depth could be achieved. Modular attachment of DNA barcode tags to the 5′-ends of the PCR primers allowed sequencing of up to 256 DNA samples in batch (FIG. 4A and FIG. 9). A median depth of 108,467 read pairs was obtained per mutation site per sample after filtering and de-multiplexing a total of 86,359,980 raw sequences generated on a single lane of an Illumina HiSeq® 2000 flow cell.

Importantly, the design of short PCR amplicons enabled us to devise a sequencing strategy that could distinguish mutant from wild-type DNA molecules with very high confidence. Illumina's paired-end sequencing mode was modified to achieve partial overlap of 75 base-pair bidirectional reads obtained sequentially from the forward and reverse strands of each clonal DNA cluster on the flow cell (FIG. 4B). Mutation hotspots were included in the overlapping sequence region so that the hotspot within each clone would be read from one strand and then proofread from the opposite strand. By discarding clones that did not have perfect sequence agreement between the two paired-end reads, the vast majority of sequencer-generated errors were eliminated. Imperfect sequence agreement was found in 22% of read pairs that had already passed Illumina's chastity filter. A median error frequency of 0.31% per base was observed when directly comparing single reads derived from either strand of wild-type control samples to known reference sequences. The frequency of such errors was reduced to 0.07% per base in the region of overlap after removing read pairs that lacked sequence consistency.

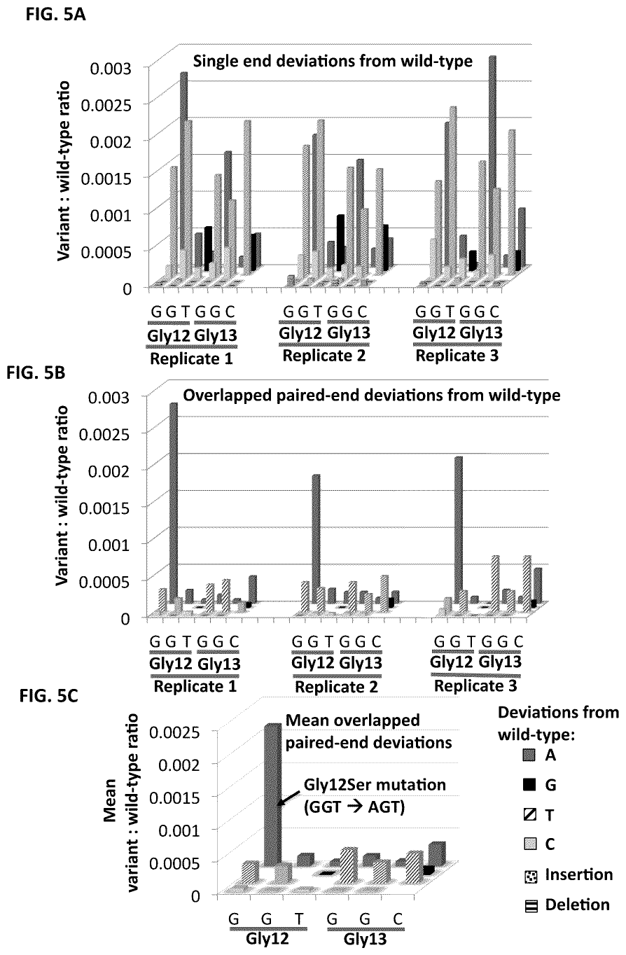

Any remaining errors were highly unlikely to be caused by coincidentally consistent misreads from opposite ends of a clone. Rather, most of these errors were probably present within the DNA molecules being sequenced, introduced by polymerase misincorporations or DNA damage. To further discriminate true mutations from such errors, all amplification and processing steps were performed in triplicate, and the mean of the three mutation counts was determined. This was done based on the premise that true mutations would be reproducibly counted in all three instances, whereas counts from randomly occurring errors would be more variable (recognizing that the distribution of errors is not entirely random). Using this approach, the frequency of miscalls of specific mutations from known wild-type samples was reduced to a median value of 0.014% (interquartile range [IQR]: 0.0052% to 0.023%; Table 3). Suppression of errors in this manner permitted rare mutations to be identified with a high degree of certainty (FIG. 5).

TABLE 3
Background level of spurious mutation counts
obtained from known wild-type samples.

| | Fraction of mutant: wild-type |
|---|---|
| Mutation Type | counts (from 3 replicate PCRs) |
| BRAF Val600Glu | 0.00013 |
| EGFR Leu858Arg | 0.000047 |
| KRAS Gly12Ser | 0.00029 |
| KRAS Gly12Val | 0.00018 |
| KRAS Gly12Arg | 0.000057 |
| KRAS Gly12Asp | 0.00015 |
| KRAS Gly12Ala | 0.000014 |
| KRAS Gly12Cys | 0.00025 |
| KRAS Gly13Ser | 0.00015 |
| KRAS Gly13Val | 0.00029 |
| KRAS Gly13Arg | 0.000050 |
| KRAS Gly13Asp | 0.000044 |
| KRAS Gly13Ala | 0.000058 |
| KRAS Gly13Cys | 0.00049 |
| Median Value | 0.00014 |
| Interquartile range | 0.000052 to 0.00023 |

### Sensitive and Accurate Quantitation of Mutant DNA

Next, mutant and wild-type DNA levels were measured over a broad range of relative concentrations. Genomic DNA from KRAS-, BRAF-, or EGFR-mutant cancer cell lines was mixed in different ratios, and then subjected to amplification and deep sequencing. Mutant DNA could be accurately and reproducibly measured in a linear manner over approximately 8 orders of magnitude and down to levels as low as 1 in 10,000 molecules (FIG. 6). Also, by testing combinations of DNA from multiple mutant cell lines, the assay was able to simultaneously quantify more than one mutation from a given sample.

Monitoring ctDNA Levels in Cancer Patients

To compare with clinical samples, plasma collected from patients with non-small cell lung cancer (NSCLC) at various times before, during, or after treatment was analyzed. Patients were enrolled in the study (and their plasma DNA was tested) without prior knowledge of the mutation status of their tumors. A total of 117 samples were obtained from 30 patients (17 patients with adenocarcinoma, 9 with undifferentiated NSCLC, and 4 with squamous cell carcinoma). KRAS Gly12Asp, Gly12Val, Gly12Cys, or Gly13Asp point-mutations were detectable in the plasma DNA of 6 patients out of 26 with adenocarcinoma or undifferentiated NSCLC. As expected, no KRAS mutations were found in specimens from patients with squamous cell carcinoma. BRAF and EGFR mutations were not detectable in any plasma samples. This was somewhat surprising for EGFR, which has a reported prevalence of activating mutations in NSCLC of approximately 10% (Lynch et al., N Engl J Med. 2004; 350: 2129-2139; Paez et al., Science. 2004; 304: 1497-1500; Pao et al., Proc. Natl. Acad. Sci. USA. 2004; 101: 13306-13311). However, evaluation of 21 available tumor tissue specimens confirmed the absence of EGFR mutations in this population (mutations occurring outside of the sequenced hotspot region may have been missed). The presence or absence of KRAS mutations in all tested tumor samples was tested to be concordant with the findings in plasma: 5 patients had identical KRAS mutations in both tumor and plasma, and 16 patients had no KRAS mutations detected from either source. Tumor tissue was unavailable or insufficient for 1 patient with mutant KRAS in the plasma, and for 4 patients with no plasma mutations. Table 4 lists the clinical characteristics and mutation findings for all enrolled patients.

TABLE 4
Clinical characteristics and mutation findings.

| | | | | | | Tumor Tissue | | | |
|---|---|---|---|---|---|---|---|---|---|
| | | | | Plasma Samples | | | | | Method of |
| Patient | | | | Mutation | No. of | NSCLC | Tissue | Tissue | Mutation |
| No. | Sex | Age | Stage | Type* | Samples | Histology | Source | Mutation | Testing |
| 1 | M | 82 | IV | KRAS | 2 | Adeno | Cells in | KRAS | Clinical |
| | | | | WT | | | pleural | WT | lab |
| | | | | EGFR | | | fluid | EGFR | |
| | | | | WT | | | | WT | |
| | | | | BRAF | | | | | |
| | | | | WT | | | | | |
| 2 | M | 68 | IV | KRAS | 2 | Adeno | Lung | Tissue not | |
| | | | | WT | | | core | available from | |
| | | | | EGFR | | | Bx | outside hospital | |
| | | | | WT | | | | | |
| | | | | BRAF | | | | | |
| | | | | WT | | | | | |
| 3 | F | 51 | IV | KRAS | 3 | Adeno | Tracheal | KRAS | Sanger |
| | | | | Gly12Asp | | | Bx | Gly12Asp | seq. |
| | | | | EGFR | | | | EGFR | |
| | | | | WT | | | | WT | |
| | | | | BRAF | | | | BRAF | |
| | | | | WT | | | | WT | |
| 4 | M | 71 | IIIB | KRAS | 12 | Squam | Para- | Not tested | |
| | | | | WT | | | tracheal | (squamous | |
| | | | | EGFR | | | lymph | histology) | |
| | | | | WT | | | node | | |
| | | | | BRAF | | | Bx | | |
| | | | | WT | | | | | |
| 5 | M | 44 | IV | KRAS | 5 | Adeno | Lung | KRAS | Sanger |
| | | | | Gly12Val | | | core | Gly12Val | seq. |
| | | | | EGFR | | | Bx | EGFR | |
| | | | | WT | | | | WT | |
| | | | | BRAF | | | | BRAF | |
| | | | | WT | | | | WT | |
| 6 | M | 68 | Lung: | KRAS | 13 | Adeno | Lung | Excess tissue not | |
| | | | IA | WT | | | core | available | |
| | | | Prost.: | EGFR | | | Bx | | |
| | | | II | WT | | | | | |
| | | | Esoph.: | BRAF | | | | | |
| | | | III | WT | | | | | |
| 7 | M | 59 | IIIA | KRAS | 8 | Squam | Lung | Not tested | |
| | | | | WT | | | core | (squamous | |
| | | | | EGFR | | | Bx | histology) | |
| | | | | WT | | | | | |
| | | | | BRAF | | | | | |
| | | | | WT | | | | | |
| 8 | F | 70 | IIIA | KRAS | 3 | Adeno | Lung | KRAS | Sanger |
| | | | | WT | | | core | WT | seq. |
| | | | | EGFR | | | Bx | EGFR | |
| | | | | WT | | | | WT | |
| | | | | BRAF | | | | BRAF | |
| | | | | WT | | | | WT | |
| 9 | M | 72 | IIIB | KRAS | 4 | Undiff | Bronchial | KRAS | Sanger |
| | | | | Gly12Val | | | brushing | Gly12Val | seq. |
| | | | | EGFR | | | | EGFR | |
| | | | | WT | | | | WT | |
| | | | | BRAF | | | | BRAF | |
| | | | | WT | | | | WT | |
| 10 | F | 62 | Lung: | KRAS | 1 | Lung | Iliac | KRAS | Sanger |
| | | | IV | WT | | Adeno | wing | WT | seq. |
| | | | Breast: | EGFR | | and | core | EGFR | |
| | | | I | WT | | Breast | Bx | WT | |
| | | | | BRAF | | Adeno | | BRAF | |
| | | | | WT | | | | WT | |
| 11 | F | 79 | IV | KRAS | 1 | Adeno | Lung | KRAS | Sanger |
| | | | | Gly12Val | | | fine | Gly12Val | seq. |
| | | | | EGFR | | | needle | EGFR | |
| | | | | WT | | | aspirate | WT | |
| | | | | BRAF | | | | BRAF | |
| | | | | WT | | | | WT | |
| 12 | M | 69 | IV | KRAS | 3 | Adeno | Scapula | KRAS | Clinical |
| | | | | WT | | | mass | WT | lab |
| | | | | EGFR | | | Bx | EGFR | |
| | | | | WT | | | | WT | |
| | | | | BRAF | | | | | |
| | | | | WT | | | | | |
| 13 | F | 61 | Lung: | KRAS | 3 | Undiff | Pre- | KRAS | Clinical |
| | | | IV | WT | | | tracheal | WT | lab |
| | | | Breast: | EGFR | | | lymph | EGFR | |
| | | | I | WT | | | node | WT | |
| | | | | BRAF | | | needle | | |
| | | | | WT | | | aspirate | | |
| 14 | M | 77 | IV | KRAS | 2 | Adeno | Calf mass | KRAS | Sanger |
| | | | | Gly12Cys | | | excision | Gly12Cys | seq. |
| | | | | EGFR | | | | EGFR | |
| | | | | WT | | | | WT | |
| | | | | BRAF | | | | BRAF | |
| | | | | WT | | | | WT | |
| 15 | M | 65 | IV | KRAS | 2 | Undiff | Bronchial | Excess tissue not | |
| | | | | Gly13Asp | | | brushing | available | |
| | | | | EGFR | | | | | |
| | | | | WT | | | | | |
| | | | | BRAF | | | | | |
| | | | | WT | | | | | |
| 16 | F | 73 | IV | KRAS | 3 | Undiff | Bronchial | KRAS | Sanger |
| | | | | WT | | | Bx | WT | seq. |
| | | | | EGFR | | | | EGFR | |
| | | | | WT | | | | WT | |
| | | | | BRAF | | | | BRAF | |
| | | | | WT | | | | WT | |
| 17 | F | 65 | IA | KRAS | 5 | Adeno | Lung | KRAS | Sanger |
| | | | | WT | | | core | WT | seq. |
| | | | | EGFR | | | Bx | EGFR | |
| | | | | WT | | | | WT | |
| | | | | BRAF | | | | BRAF | |
| | | | | WT | | | | WT | |
| 18 | F | 77 | IV | KRAS | 1 | Adeno | Lung | Excess tissue not | |
| | | | | WT | | | core | available | |
| | | | | EGFR | | | Bx | | |
| | | | | WT | | | | | |
| | | | | BRAF | | | | | |
| | | | | WT | | | | | |
| 19 | F | 75 | IV | KRAS | 2 | Adeno | Bronchial | KRAS | Clinical |
| | | | | WT | | | Bx | WT | lab |
| | | | | EGFR | | | | EGFR | |
| | | | | WT | | | | WT | |
| | | | | BRAF | | | | | |
| | | | | WT | | | | | |
| 20 | M | 73 | IB | KRAS | 5 | Squam | Lung | Not tested | |
| | | | | WT | | | lobec- | (squamous | |
| | | | | EGFR | | | tomy | histology) | |
| | | | | WT | | | | | |
| | | | | BRAF | | | | | |
| | | | | WT | | | | | |
| 21 | M | 73 | IIB | KRAS | 4 | Adeno | Lung | KRAS | Sanger |
| | | | | WT | | | core | WT | seq. |
| | | | | EGFR | | | Bx | EGFR | |
| | | | | WT | | | | WT | |
| | | | | BRAF | | | | BRAF | |
| | | | | WT | | | | WT | |
| 22 | F | 68 | IV | KRAS | 3 | Undiff | Lung | KRAS | Clinical |
| | | | | WT | | | tumor | WT | lab |
| | | | | EGFR | | | excision | EGFR | |
| | | | | WT | | | | WT | |
| | | | | BRAF | | | | | |
| | | | | WT | | | | | |
| 23 | F | 79 | IA | KRAS | 3 | Undiff | Lung | Tissue not | |
| | | | | WT | | | core | available from | |
| | | | | EGFR | | | Bx | outside hospital | |
| | | | | WT | | | | | |
| | | | | BRAF | | | | | |
| | | | | WT | | | | | |
| 24 | M | 64 | IIIB | KRAS | 8 | Squam | Lung | Not tested | |
| | | | | WT | | | core | (squamous | |
| | | | | EGFR | | | Bx | histology) | |
| | | | | WT | | | | | |
| | | | | BRAF | | | | | |
| | | | | WT | | | | | |
| 25 | F | 73 | Locally | KRAS | 8 | Undiff | Lung | KRAS | Sanger |
| | | | recur. | WT | | | lobec- | WT | seq. |
| | | | IB | EGFR | | | tomy | EGFR | |
| | | | | WT | | | | WT | |
| | | | | BRAF | | | | BRAF | |
| | | | | WT | | | | WT | |
| 26 | F | 63 | IIIB | KRAS | 1 | Adeno | Lung | KRAS | Sanger |
| | | | | WT | | | core | WT | seq. |
| | | | | EGFR | | | Bx | EGFR | |
| | | | | WT | | | | WT | |
| | | | | BRAF | | | | BRAF | |
| | | | | WT | | | | WT | |
| 27 | F | 74 | IIIA | KRAS | 4 | Undiff | Para- | KRAS | Sanger |
| | | | | WT | | | tracheal | WT | seq. |
| | | | | EGFR | | | lymph | EGFR | |
| | | | | WT | | | node | WT | |
| | | | | BRAF | | | Bx | BRAF | |
| | | | | WT | | | | WT | |
| 28 | F | 61 | IV | KRAS | 3 | Adeno | Spine | KRAS | Sanger |
| | | | | WT | | | Met Bx | WT | seq. and |
| | | | | EGFR | | | | EGFR | Clinical |
| | | | | WT | | | | WT | lab |
| | | | | BRAF | | | | BRAF | |
| | | | | WT | | | | WT | |
| 29 | F | 82 | IV | KRAS | 2 | Undiff | Lung | KRAS | Sanger |
| | | | | WT | | | core | WT | seq. and |
| | | | | EGFR | | | Bx | EGFR | Clinical |
| | | | | WT | | | | WT | lab |
| | | | | BRAF | | | | BRAF | |
| | | | | WT | | | | WT | |
| 30 | F | 69 | Lung: | KRAS | 1 | Adeno | Lung | KRAS | Sanger |
| | | | IV | WT | | | fine | WT | seq. |
| | | | Breast: | EGFR | | | needle | EGFR | |
| | | | IV | WT | | | aspirate | WT | |
| | | | | BRAF | | | | BRAF | |
| | | | | WT | | | | WT | |

The list is ordered by date of first specimen collection.
*Plasma DNA was only tested for mutations at codons 12 and 13 of KRAS, 858 of EGFR, and 600 of BRAF.
Squam = Squamous cell carcinoma
Adeno = Adenocarcinoma
Undiff = Undifferentiated NSCLC (not otherwise specified)
WT = Wild-type
Bx = Biopsy
Sanger Seq. = Direct Sanger sequencing of tissue-derived PCR amplicons by our laboratory.
Clinical lab = Mutations tested for clinical purposes in a laboratory certified under the Clinical Laboratory Improvement Amendments of 1988 (CLIA). Tissue was not tested for BRAF mutations by clinical laboratories because of low prevalence in NSCLC.

For patients with detectable plasma DNA mutations, changes in measured ctDNA levels were followed in the context of therapeutic interventions or disease progression. To determine the absolute concentration of mutant KRAS DNA fragments in a plasma sample, the total concentration of KRAS fragments was measured by real-time PCR and then multiplied by the fraction of mutant molecules determined by deep sequencing. The median concentration among samples with detectable mutations was 5,694 mutant KRAS molecules per mL (IQR: 2,655 to 25,123). Time-courses of mutant ctDNA measurements for patients who had 3 or more samples collected are shown in FIG. 7 (data for patients with fewer measurements are shown in FIG. 8). In two cases, the ctDNA level decreased upon treatment with radiation and/or systemic therapy. Aggressive progression of metastatic disease in a different patient was accompanied by a substantial rise in ctDNA. In another two cases, ctDNA levels increased shortly after initiating treatment, perhaps because more tumor DNA was released into the bloodstream as cancer cells were being killed.

### Example 2

This example includes methods that incorporate elements of Example 1, but also includes several modifications. (FIGS. 10 and 11). In this example, 40 different genomic target regions were analyzed. Of the 40 genomic target regions, 38 were prone to developing somatic mutations, and 2 were included as controls that were not expected to be mutated.

### Preparation of Mixtures of Primers Having Combinations of Modular Oligonucleotide Segments

As described previously, early tagging of targeted DNA template molecules required the production of mixtures of primers having a common barcode in their 5′ region, and having several different gene-specific primer segments at their 3′ end. Herein modular oligonucleotide segments were combined during oligonucleotide synthesis on an automated synthesizer, “modular automated synthesis and purification”, and the approach is illustrated in (FIG. 3).

Each different gene-specific 3′-portion was synthesized on separate oligonucleotide synthesis columns. Standard phosphoramidite chemistry was used, and the oligonucleotides were grown on a solid support. Both polystyrene and controlled-pore-glass were used as solid supports, but polystyrene was preferable. Both types of supports performed similarly. The solid support consisted of small particles that appeared as a powder. The powder was contained within an oligonucleotide synthesis column, sandwiched loosely between two frits. Multiple different 3′-segments were grown (oligomerized by chemical coupling of phosphoramidite monomers) in separate synthesis columns on an automated synthesizer in the 3′ to 5′ direction. The synthesis was paused, and partially synthesized oligonucleotides were left on the column in the protected state with the trityl group left on.

“Pipette tip”-style oligonucleotide synthesis columns were utilized with sufficient controlled-pore glass (1000 angstrom pore size) or polystyrene to synthesize oligos at the 40 nanomole or 200 nanomole scale (3-Prime, Aston, PA). Forty different partial 3′ oligonucleotide segments were synthesized on 40 separate columns using a Dr. Oligo 192 automated synthesizer. The oligonucleotides were not cleaved from the solid supports, were not deprotected, and the trityl group was left on so that further synthesis could be continued. The sequences of these 40 different 3′ segments are listed in Table 5.

TABLE 5
List of forty 3′ oligonucleotide segments synthesized in separate
columns for first phase of modular automated synthesis.

| | | SEQ |
|---|---|---|
| Name | DNA Sequence | ID NO: |
| 3′ segment1 | AGACGTGTGCTCTTCCGATCTNNNNNNCTGTGCTGTGACT | 52 |
| | GCTTG | |
| 3′ segment2 | AGACGTGTGCTCTTCCGATCTNNNNNNTAGCACATGACGG | 53 |
| | AGGTT | |
| 3′ segment3 | AGACGTGTGCTCTTCCGATCTNNNNNNACAAATACTCCAC | 54 |
| | ACGCAAATT | |
| 3′ segment4 | AGACGTGTGCTCTTCCGATCTNNNNNNATATTTGGATGAC | 55 |
| | AGAAACACTT | |
| 3′ segment5 | AGACGTGTGCTCTTCCGATCTNNNNNNCTGTGATGATGGT | 56 |
| | GAGGATGG | |
| 3′ segment6 | AGACGTGTGCTCTTCCGATCTNNNNNNCTGGGACGGAACA | 57 |
| | GCTTTGAG | |
| 3′ segment7 | AGACGTGTGCTCTTCCGATCTNNNNNNTGCAATTTCTACA | 58 |
| | CGAGATCCTCT | |
| 3′ segment8 | AGACGTGTGCTCTTCCGATCTNNNNNNTCTTTGGAGTATTT | 59 |
| | CATGAAACAAATGA | |
| 3′ segment9 | AGACGTGTGCTCTTCCGATCTNNNNNNAACAGTAAAAATA | 60 |
| | GGTGATTTTGGTCTA | |
| 3′ segment10 | AGACGTGTGCTCTTCCGATCTNNNNNNTGCAACTACTGGA | 61 |
| | CGCTGGAC | |
| 3′ segment11 | AGACGTGTGCTCTTCCGATCTNNNNNNCTCAATTTTGTTTC | 62 |
| | AGGACCTGCT | |
| 3′ segment12 | AGACGTGTGCTCTTCCGATCTNNNNNNCTGGCAGCAACAG | 63 |
| | TCTTACCT | |
| 3′ segment13 | AGACGTGTGCTCTTCCGATCTNNNNNNACCCAGCTTG | 64 |
| | GAGGCTGC | |
| 3′ segment14 | AGACGTGTGCTCTTCCGATCTNNNNNNAGCCAGGCCGCTG | 65 |
| | AAGACA | |
| 3′ segment15 | AGACGTGTGCTCTTCCGATCTNNNNNNGGCAATTCACTGT | 66 |
| | AAAGCTGGAAAG | |
| 3′ segment16 | AGACGTGTGCTCTTCCGATCTNNNNNNATGAAGATATATT | 67 |
| | CCTCCAATTCAGGAC | |
| 3′ segment17 | AGACGTGTGCTCTTCCGATCTNNNNNNGCGTTTCCTTTAA | 68 |
| | CCACATAATTAGAATC | |
| 3′ segment18 | AGACGTGTGCTCTTCCGATCTNNNNNNGTTTTCCCTTTCTC | 69 |
| | CCCACAG | |
| 3′ segment19 | AGACGTGTGCTCTTCCGATCTNNNNNNGTTCCTGTAGCAA | 70 |
| | AACCAGAAATC | |
| 3′ segment20 | AGACGTGTGCTCTTCCGATCTNNNNNNCGGTGAGAAAGTT | 71 |
| | AAAATTCCCGTC | |
| 3′ segment21 | AGACGTGTGCTCTTCCGATCTNNNNNNAAGCATGTCAAGA | 72 |
| | TCACAGATTTTG | |
| 3′ segment22 | AGACGTGTGCTCTTCCGATCTNNNNNNCTCACCTCCACCG | 73 |
| | TGCAGCT | |
| 3′ segment23 | AGACGTGTGCTCTTCCGATCTNNNNNNGACCACCCGCACG | 74 |
| | TCTGT | |
| 3′ segment24 | AGACGTGTGCTCTTCCGATCTNNNNNNTCTTCCATACTTG | 75 |
| | ATTCATGATATTTTACT | |
| 3′ segment25 | AGACGTGTGCTCTTCCGATCTNNNNNNGACCTCCTCAAAC | 76 |
| | AGCTCAAAC | |
| 3′ segment26 | AGACGTGTGCTCTTCCGATCTNNNNNNATGGGAGATCTTC | 77 |
| | ACGCTGG | |
| 3′ segment27 | AGACGTGTGCTCTTCCGATCTNNNNNNTCCCTGAGCGTCA | 78 |
| | TCTGCC | |
| 3′ segment28 | AGACGTGTGCTCTTCCGATCTNNNNNNCGCTGGTGGAGGC | 79 |
| | TGACGA | |
| 3′ segment29 | AGACGTGTGCTCTTCCGATCTNNNNNNGTTCCCTATCAAA | 80 |
| | TATGTCAACGACT | |
| 3′ segment30 | AGACGTGTGCTCTTCCGATCTNNNNNNAATTTTGGTCTTG | 81 |
| | CCAGAGACA | |
| 3′ segment31 | AGACGTGTGCTCTTCCGATCTNNNNNNTATCGACTCCACC | 82 |
| | GAGGTCA | |
| 3′ segment32 | AGACGTGTGCTCTTCCGATCTNNNNNNATACTTGGAGGAC | 83 |
| | CTGCACG | |
| 3′ segment33 | AGACGTGTGCTCTTCCGATCTNNNNNNGTCGTCAAGGCAC | 84 |
| | TCTTGCCT | |
| 3′ segment34 | AGACGTGTGCTCTTCCGATCTNNNNNNCGATATTCTCGAC | 85 |
| | ACAGCAGGT | |
| 3′ segment35 | AGACGTGTGCTCTTCCGATCTNNNNNNATCAGTGCGCTTT | 86 |
| | TCCCA | |
| 3′ segment36 | AGACGTGTGCTCTTCCGATCTNNNNNNTGACATACTGGAT | 87 |
| | ACAGCTGGA | |
| 3′ segment37 | AGACGTGTGCTCTTCCGATCTNNNNNNGTGGTCAGCGCAC | 88 |
| | TCTTGCCC | |
| 3′ segment38 | AGACGTGTGCTCTTCCGATCTNNNNNNTCATCCTGGATAC | 89 |
| | CGCCGGC | |
| 3′ segment39 | AGACGTGTGCTCTTCCGATCTNNNNNNATCCTGTTTATAA | 90 |
| | TATTGACAAAACACCT | |
| 3′ segment40 | AGACGTGTGCTCTTCCGATCTNNNNNNATCAGGACAAAGT | 91 |
| | CCGGATTGA | |

These oligonucleotides were synthesized at the 200 nanomole scale, with the oligo left on the column in the protected state with the trityl group left on. Positions marked “N” have equal probability of being A, C, G, or T.

The solid supports of all 40 partially synthesized oligonucleotides were dried by blowing argon gas through the columns, and then the controlled-pore glass or polystyrene powder from all 40 columns was mixed by pouring the contents of each column (after cutting the tops off of the columns) into a common container (such as a glass vial). The solid support particles were then suspended in a solvent of similar density so that the particles could be thoroughly mixed and then the mixture could be dispensed into fresh oligonucleotide synthesis columns. When using polystyrene supports, a 3:1 mixture of dichloromethane: acetonitrile was used as the suspension liquid, and when using controlled-pore glass supports, a 5:1 mixture of 1,2-dibromoethane: acetonitrile was used as the suspension liquid. The particles were maintained as a uniform slurry in the liquid by constantly swirling or agitating the vial while using a pipette to dispense equal volumes of the slurry into fresh columns (with the bottom frit already in place). The slurry was dispensed into 96 fresh columns. The particles settled onto the frits, while the liquid drained out from the bottom of the columns by gravity. To ensure that the particles had all settled onto the frit, the columns were filled with acetonitrile and this was again allowed to drain out from the bottom by gravity. After the acetonitrile had fully drained out, the top frits were put in place to secure the powder into the columns.

The new columns were then placed back on the automated synthesizer, and the oligonucleotide synthesis was continued. Each column was assigned a different barcode sequence that was incorporated into the 5′ oligonucleotide segment. A “dummy base” was added to the 3′ end of the 5′ segment sequence when programming the synthesizer in order to account for the partially synthesized oligonucleotides that were already present on the solid supports. The sequences of the 96 different 5′ segments consisted of the following common sequence with each of 96 different barcodes inserted in the position marked [BC1-96]. One unique barcode was used per oligonucleotide synthesis column.

(SEQ ID NO: 92)
5′-CGAGACGGATCAAGCAGAAGACGGCATACGAGATNN[BC1-
96]GTGACTGGAGTTC(T)-3′

The “T” in parentheses at the 3′-end of the sequence is the “dummy base”. The 96 barcodes that were used are listed in Table 6. The automated synthesizer was programmed to carry out synthesis at a 40 nmole scale (which determines the volume of reagents passed through the columns), although the actual amount of solid support in each column was likely to produce less than 40nmoles of oligonucleotides.

TABLE 6
List of 96 sample-specific barcodes

| | Sequence |
|---|---|
| Barcode # | (BBBBBBBB) |
| 1 | CCGATATT |
| 2 | GCCATATT |
| 3 | TCTGGATT |
| 4 | ACTCGATT |
| 5 | TCACGATT |
| 6 | ACTGCATT |
| 7 | TCAGCATT |
| 8 | TCTCCATT |
| 9 | ATTGGTGT |
| 10 | TTAGGTGT |
| 11 | ATACGTGT |
| 12 | ATAGCTGT |
| 13 | ATTCCTGT |
| 14 | TTACCTGT |
| 15 | CTCTATGT |
| 16 | CTCTTAGT |
| 17 | CTGATAGT |
| 18 | GTCATAGT |
| 19 | ATAGGAGT |
| 20 | ATTCGAGT |
| 21 | TTACGAGT |
| 22 | ATTGCAGT |
| 23 | TTAGCAGT |
| 24 | ATACCAGT |
| 25 | AGTGGTCT |
| 26 | TGAGGTCT |
| 27 | TGTCGTCT |
| 28 | TGTGCTCT |
| 29 | AGTCCTCT |
| 30 | TGACCTCT |
| 31 | CGCTATCT |
| 32 | CGCTTACT |
| 33 | CGGATACT |
| 34 | GGCATACT |
| 35 | TGTGGACT |
| 36 | AGTCGACT |
| 37 | TGACGACT |
| 38 | AGTGCACT |
| 39 | TGAGCACT |
| 40 | TGTCCACT |
| 41 | TCATTGTG |
| 42 | TCTATGTG |
| 43 | ACTATCTG |
| 44 | ACTTACTG |
| 45 | TCATACTG |
| 46 | ATTATCGG |
| 47 | TTATACGG |
| 48 | TGATTGCG |
| 49 | TGTATGCG |
| 50 | AGTATCCG |
| 51 | AGTTACCG |
| 52 | TGATACCG |
| 53 | ACTATGTC |
| 54 | ACTTAGTC |
| 55 | TCATAGTC |
| 56 | TCATTCTC |
| 57 | TCTATCTC |
| 58 | ATTATGGC |
| 59 | TTATAGGC |
| 60 | TTATTCGC |
| 61 | AGTATGCC |
| 62 | AGTTAGCC |
| 63 | TGATAGCC |
| 64 | TCTGGTTA |
| 65 | TCACGTTA |
| 66 | TCAGCTTA |
| 67 | TCTCCTTA |
| 68 | CCGTATTA |
| 69 | GCCTATTA |
| 70 | CCGTTATA |
| 71 | GCCTTATA |
| 72 | TCAGGATA |
| 73 | TCTCGATA |
| 74 | TCTGCATA |
| 75 | TCACCATA |
| 76 | CTGATTGA |
| 77 | GTCATTGA |
| 78 | TTACGTGA |
| 79 | TTAGCTGA |
| 80 | CTGTATGA |
| 81 | GTCTATGA |
| 82 | CGGATTCA |
| 83 | GGCATTCA |
| 84 | TGTGGTCA |
| 85 | TGACGTCA |
| 86 | TGAGCTCA |
| 87 | TGTCCTCA |
| 88 | CGGTATCA |
| 89 | GGCTATCA |
| 90 | CGGTTACA |
| 91 | GGCTTACA |
| 92 | CGCATACA |
| 93 | TGAGGACA |
| 94 | TGTCGACA |
| 95 | TGTGCACA |
| 96 | TGACCACA |

After completion of the second phase of the modular synthesis, the oligos were cleaved off the solid supports with the trityl group still left on. They underwent rapid deprotection followed by purification on a separate Glen-Pak DNA reverse-phase cartridge for each of the 96 oligonucleotide mixtures (Glen Research, Sterling, VA). The trityl group at the 5′-end of completed oligonucleotides was selectively retained by the cartridge, enriching for full-length products and removed failure sequences that did not contain the trityl group. The trityl group was removed upon completion of purification. The purified oligonucleotides were then dried and re-suspended in 10 mM Tris pH 7.6 to produce a 33 micromolar working stock solution. Polyacrylamide gel purification was used in some cases to further purify the full-length oligonucleotides.

### Collection and Processing of Patient Plasma Samples

Blood was collected by venipuncture into a vacuum tube containing potassium-EDTA. Various tube sizes were used, typically between 3 mL and 10 mL. Blood was inverted in the tube several times at the time of collection to ensure even mixing of the K2-EDTA. Samples were stored temporarily and transported at room temperature (20-25° C.) prior to separation of plasma. Plasma was separated and frozen as soon as possible after blood collection, preferably within 3 or 4 hours. The collection tubes were centrifuged at 1000× g for 10 minutes in a clinical centrifuge with a swinging bucket rotor with slow acceleration and deceleration (brake off). Plasma was removed from the red blood cells and buffy coat using a 1 mL pipette, being careful not to disturb the cells at the bottom of the tube (to avoid aspirating white blood cells which would lead to increased background wild-type DNA levels). The plasma was dispensed into 1.5 mL cryovials in 0.5 to 1 mL aliquots. The plasma was then frozen at −80° C. until needed for further processing.

### Extraction and Purification of DNA From Plasma

Plasma was removed from the −80° C. freezer and was thawed at room temperature for 15 to 30 minutes before proceeding with DNA extraction. Thawed plasma was then centrifuged at 6800× g for 3 minutes to remove any cryoprecipitate. The supernatant was transferred to a fresh tube for further processing.

The QiaAmp® DNA Blood Mini Kit (Qiagen) was used for purification from plasma volumes up to 200 μL (elution volume of 50 μL), and the QiaAmp® MinElute® Virus Vacuum Kit (Qiagen) has also been used for plasma volumes up to 1 mL (elution volume as low as 20 μL). For larger volumes of a particular sample of plasma, more than one column of the QiaAmp® MinElute® Virus Vacuum Kit was used for purification. All kits were used according to the manufacturer's instructions, generally eluting the DNA into the lowest recommended volume (preferably 20 μL). To process 1 mL of plasma using the QiaAmp® MinElute® Virus Vacuum Kit, 5 micrograms of carrier RNA (cRNA; Qiagen) were added per mL, and the user-developed protocol found on the Qiagen website was followed.

### Primer-Extension Reaction

Specific mutation-prone regions of purified, plasma-derived template DNA molecules were copied using targeted gene-specific primers. The number of different gene-specific primer sequences used in each tube depended on the number of targeted DNA regions within the genome. A combination of 40 different gene-specific primers were used in each sample to target 40 different gene regions. As described previously, each set of gene-specific primers had a unique, sample-specific DNA sequence (a barcode) near the 5′-end of the primers that were incorporated in a modular fashion. Each sample underwent primer-extension using an approximately equimolar concentration of 40 different gene-specific primers, all of which had the same sample-specific barcode. These primers also included degenerate sequence regions known as molecular lineage tags (MLTs) as well as common sequences at the 5′-end that allowed for hybridization of “universal” PCR primers in subsequent steps.

Control DNA molecules containing known mutations were spiked into each primer extension reaction to serve as internal quantitative standards. These DNA molecules were cartridge-purified oligonucleotides that were synthesized to contain variations from the wild-type sequence at two distinct positions (which would be extremely unlikely to occur in plasma-derived DNA). These variations allowed the control sequences to be readily distinguished from other variants within DNA purified from a clinical sample. The sequences of the top strands of these control DNA oligonucleotides are listed in Table 7. Reverse complements of these 40 sequences were also separately synthesized to produce bottom strands. In order to make the control DNA as similar as possible to the clinically-derived DNA, both strands were annealed to make them double-stranded before adding them to the primer-extension reaction. The double-stranded DNA was quantified by UV spectrometry and then diluted to the desired concentration. To each primer-extension reaction, approximately 200 copies of the double-stranded control DNA fragments corresponding to each of the 40 gene target sites were added.

TABLE 7
List of spiked-in quantitative standard oligonucleotides containing
mutations at 2 distinct sites relative to wild-type.

| | | SEQ ID |
|---|---|---|
| Name | DNA Sequence | NO: |
| CFTP-1 | TCAACAAGATGTTTTGCCAACTGGCCAAGACCTGCCC | 93 |
| | TGTGCAGCTGTGGGTTGATTCCACACCCCCGCCCGGC | |
| | ACCCGCGTCCGCGTCATGACCATCTACAAGCAGTCAC | |
| | AGCACATGA | |
| CFTP-2 | TCACAGCACATGACGGAGGTTGTGAGGCGCTGCCACC | 94 |
| | ACCATGTGCGCTGCTCAGATAGCGATGGTGAGCAGCT | |
| | GGGGCTGGAGAGACGACAGGGCTG | |
| CFTP-3 | ACAGGGCTGGTTGCCCAGGGTCCCCAGGCCTCTGATT | 95 |
| | CCTCACTGATTGCTCTTAGGTCTGGCCCCTCCTCAGCA | |
| | TCATATCCGAGTCGAAGGAAATTTGCGTGTGGAGTAT | |
| | TTGGATG | |
| CFTP-4 | GAGTATTTGGATGACAGAAACACTTTTCGACACAGTG | 96 |
| | TGGTGATGCCCTATGAGCCGCCTGAGGTCTGGTTTGC | |
| | AACTGGGGTCTCTGGGAGGAGGGGTTAAGGGTGGTT | |
| | GTCAGTGGCCCTC | |
| CFTP-5 | CTGGCCTCATCTTGGGCCTGTGTTATCTCCTAGGTTGG | 97 |
| | CTCTGACTGTACCACCATCCACTACAACGACATGTGT | |
| | AACTGTTCCTGCATGGGCGGCATGAACCGGAGGCCCA | |
| | TCCTCACCATCATCACACTGG | |
| CFTP-6 | TACTGGGACGGAACAGCTTTGAGGTGCGTGTTTGTGC | 98 |
| | ATGTCCTGGGACAGACCGGCGCACAGAGGAAGAGAA | |
| | TCTCCGCAAGAAAGGGGAGCCTCACCACGAGCTGCC | |
| | CCCAG | |
| CFPIK-1 | CAAAGCAATTTCTACACGAGATCCTCTCTCTGAAGTC | 99 |
| | AGTGAGCAGGAGAAAGATTTTCTATGGAGTCACAGG | |
| | TAAGTGCTAAAATGGAGATTCTCTGTTTCTTTTTC | |
| CFPIK-2 | GAGGCTTTGGAGTATTTCATGAAACAAATGAATCATA | 100 |
| | CACATCATGGTGGCTGGACAACAAAAATGGATTGGA | |
| | TCTTCCACACAATTAAACAGCATGCATTGAACTGAAA | |
| | AG | |
| CFBRAF | CCTCACAGTAAAAATAGGTGATTTTGGTCTAGCGACA | 101 |
| | GTGAAAGCTCGATGGAGTGGGTCCCATCAGTTTGAAC | |
| | AGTTGTCTGGATCCATTTTGTGGATGGTAAGAATT | |
| CFFox | AAGGGCAACTACTGGACGCTGGACCCGACCTGCGCA | 102 |
| | GACATGTTCGAGAAGGGCAACTACCGGCGCCGCCGC | |
| | CGCATGAAGAGGCCCTTCCGGCCG | |
| CFGNAS | ACCTCAATTTTGTTTCAGGACCTGCTTCACTGCCGTAT | 103 |
| | CCTGACTTCTGGAATCTTTGAGACCAAGTTCCAGGTG | |
| | GACAAAGTCAACTTCCAGTAAGCCAACT | |
| CFCTNN | CACTGGCAGCAACAGTCTTACCTGGACTCTGGAATCC | 104 |
| | ATTCTGATGCCACTACCACAGATCCTTCTCTGAGTGG | |
| | TAAAGGCAATCCTGAGGAAGAGGATGTGGATACCTC | |
| | CCAAGTCCTGTAT | |
| CFPPP-1 | CTGCCTGCTGCCTCAGGATCCCCGTCCCCGACTCCCA | 105 |
| | GGTACTTCCGGAACCTGTGCTCAGATGACACCCCCAC | |
| | GGTGCGGCGGACCGCAGCCTCCAAGCTGGGGGAG | |
| CFPPP-2 | CTGCGCCAGGCCGCTGAAGACAAGACCTGGCGCATC | 106 |
| | CGCTACATGGTGGCTGACAAGTTCACAGAGGTAGATG | |
| | AGCGACCGTTGACATTGTCCCACTGGT | |
| CFPTEN-1 | TGCAGCAATTCACTGTAAAGCTGGAAAGGGACGAAC | 107 |
| | AGGTGTAATGACATGTGCATATTTATTACATCGGGGC | |
| | AAATTTTTAAAGGCACAAGAGGCCCTAGATTTCTATG | |
| | GGGAAG | |
| CFPTEN-2 | AGGTGAAGATATATTCCTCCAATTCAGGACCCTCACG | 108 |
| | ACGGGTAGACAAGTTCATGTACTTTGAGTTCCCTCAG | |
| | CCGTTACCTGTGTGTGGTGATATCAAAGTAGAGTTCT | |
| CFKIT-1 | GAGACTTGGCAGCCAGAAATATCCTCCTTACTCATGG | 109 |
| | TCGGATCACAAAGATTTGTGATTTTGGTCTAGCCATA | |
| | GACATCACGAATGATTCTAATTATGTGGTTAAAGGAA | |
| | ACGTGAG | |
| CFKIT-2 | TATTTTTCCCTTTCTCCCCACAGAAACCTATGTATGAA | 110 |
| | GTACAGTGGAAGGATGTTGAGGAGATAAATGGAAAC | |
| | AATTATGTTTACATAGACCCAACACAACTTCCTTATG | |
| | ATCACAAATGGGAGTTTC | |
| CFKIT-3 | GTTTTCCTGTAGCAAAACCAGAAATCCTGACTTACGA | 111 |
| | CAGGCTAGTGAATGGCATGCTCCAATGTGTGGCAGCA | |
| | GGATTCCCAGAGCCCACAATAGATTGGTATTTTT | |
| CFEG-1 | AGAAGGTGAGAAAGTTAAAATTCCCGTCGCTATGAA | 112 |
| | GGAATTAAGAGAAGCAACATCTCCGTAAGCCAACAA | |
| | GGAAATCCTCGATGTGAGTTTCTGCTTTGCTGTGTGG | |
| | GGGTC | |
| CFEG-2 | CCGCAGCATGTCAAGATCACAGATTTTGGGCTGGACA | 113 |
| | AACAGCTGGGTGCGGAAGAGAAAGAATACCATGCAG | |
| | AAGGAGGCAAAGTAAGGAGGTGGCTTTAG | |
| CFEG-3 | GCCTCACCTCCACCGTGCAGCTCATGACGTAGCTCAT | 114 |
| | GCCCTTCGGCTGCCTCCTGGACTATGTCCGGGAACAC | |
| | AAAGACAATA | |
| CFAKT1 | TCTCACCACCCGCACGTCTGTAGAGGACTACATCAAG | 115 |
| | ACCTGGCGGCCACGCTACTTCCTCCTCAAGAATGATG | |
| | GCACCTTCATTGG | |
| CFATM | TGTACTTCCATACTTGATTCATGATATTTTACTCCTAG | 116 |
| | ATACGAATGAATCATGGAGAAATCTGCTTTCTACACA | |
| | TGTTCAGGGATTTTTCACCAGCTGTCTTCGACACTTCT | |
| | CGC | |
| CFAPC | CACCACCTCCTCAAACAGCTCAAACCATGCGATAAGT | 117 |
| | ACCTAAAAATAAAGCACCTACTGCTGAAAAGAGAGA | |
| | GAGTGGACCTAAGCAAGCTGCAGT | |
| CFFGFR-1 | GCTCTGGGAGATCTTCACGCTGGGGGACTCCCCGTAT | 118 |
| | CCCGGCATCCCTGTGGAGGAGCTCTTCAAGCTGCTGA | |
| | AGGAGGGCCACCGCATGGACAAGCCCGCCA | |
| CFFGFR-2 | TGGCCCCTGAGCGTCATCTGCCCCCACTGAGCGCTCC | 119 |
| | ACGCACCGGCCCATCCTGCAGGCGGGGCTGCCGGCC | |
| | AACCAGACGGCGGTGCTGGGCAGCGACGTGGAGTTC | |
| | C | |
| CFFGFR-3 | AGGAGCTGGTGGAGGCTGACGAGGCGGGCAGTATGT | 120 |
| | ATACAGGCATCCTCAGCTACGGGGTGGGCTTCTTCCT | |
| | GTTCATCCTGGTGGTGGCGGCTGTGAC | |
| CFMET-1 | GCATTCCCTATCAAATATGTCAACGACTTCATCAACA | 121 |
| | AGATAGTCAACAAAAACAATGTGAGATGTCTCCAGC | |
| | ATTTTTACGGACCCAATCATGAGCACTGCTTTAATAG | |
| | GGTAA | |
| CFMET-2 | GCTGATTTTGGTCTTGCCAGAGACATGTATCATAAAC | 122 |
| | AATACTATAGTGTACACAACAAAACAGGTGCAAAGC | |
| | TGCCAGTGAAGTGGATGGCTTTGGAAAGTCTG | |
| CFSTK-1 | CCGCATCGACTCCACCGAGGTCATCTACCAGCCGAGC | 123 |
| | CGCATGCGGGCCAAGCTCATCGGCAAGTACCTGATGG | |
| | GGGACCTGCTGGGGGAAGGCTCTTACGGCAAGGTGA | |
| | AGGAGGTGCTGGACTCGGAG | |
| CFSTK-2 | CCGTACTTGGAGGACCTGCACGGCGCGGATGAGGAC | 124 |
| | GAGGACCACTTCGACATCGAGGATGACATCATCTACA | |
| | CTCAGGACTTCACGGTGCCCGGTGAGTCTGGCGGGGG | |
| CFKRAS-1 | TATAGTCACATTTTCATTATTTTTATTATAAGGCCTGC | 125 |
| | TGAAAATGACTGAATATAAACTTGTGGTAGTTGCAGA | |
| | TGGTGGCGTAGGCAAGAGTGCCTTGACGATAC | |
| CFKRAS-2 | CTTGGATATTCTCGACACAGCAGGTCAAGACGAGTAC | 126 |
| | TGTGCAATGAGGGACCAGTACATGAGGACTGGGGAG | |
| | GGCTTTCTTTGTGTATTTGCCATAAATAATACTAAA | |
| CFNRAS-1 | TGTAGATGTGGCTCGCCAATTAACCCTGATTACTGGT | 124 |
| | TTCCAACAGGTTCTTGCTGGTGTGAAATGACTGAGTA | |
| | CAAACTGGTCGTGGATGGAGCAGGTGGTGTTGGGAA | |
| | AAGCGCACTGACAAT | |
| CFNRAS-2 | GTTGGACATACTGGATACAGCTGGACAAGAAGAGCA | 128 |
| | CAGTGACATGAGAGACCAATACATGAGGACAGGCGA | |
| | AGGCTTCCTCTGTGTATTTGCCATCAATAATAGCAAG | |
| | TCAT | |
| CFHRAS-1 | GGTGGGGCAGGAGACCCTGTAGGAGGACCCCGGGCC | 129 |
| | GCAGGCCCCTGAGGAGCGATGACGGAATATAAGCTG | |
| | GTGGTCGTGGACGCCGGCGGTGTGGGCAAGAGTGCG | |
| | CTGACCATCC | |
| CFHRAS-2 | TGGACATCCTGGATACCGCCGGCCAGGAGTACTACAG | 130 |
| | CGCCATGCGGGACCAGTACATGCGCACCGGGGAGGG | |
| | CTTCCTGTGTGTGTTTGCCATCAACAACACCAAGTCTT | |
| | TTGAGGA | |
| CFK-Ctrl | TGTTCCTGTTTATAATATTGACAAAACACCTTAGCGG | 131 |
| | ATGACATTTAAGAATTCTAAAAGTCCTAATATATGTA | |
| | ATATATATTCAGTTGCCTGAAGAGAAACATAAAGAAT | |
| | CCTTTCTTAAT | |
| CFB-Ctrl | ATGTCAGGACAAAGTCCGGATTGAATATAACTCTGCT | 132 |
| | TTATATTATAGGCCTATGAAGAATACACCAGCAAGCT | |
| | AGATGCACTCCAACAAAGAGAACAACAGTTATTGGA | |
| | ATCTCTGGG | |

*All oligos synthesized at 40 nmole scale with cartridge purification.

Conditions were optimized so that on average, more than one copy of each original DNA template molecule would be present at the beginning of the next amplification step. Typically between 2 and 10 cycles of primer-extension were carried out. Primer extension was performed using Accuprime Taq polymerase (Invitrogen) as described below.

### Primer-Extension Reaction Setup (30 μL Reaction)

| Purified template DNA (with co-eluted | 20 μL (or less) | |
|---|---|---|
| carrier RNA [cRNA]) | | |
| 100 copies of control mutant DNA in | (as needed) | |
| 10 mM Tris (with 300 ng per mL cRNA) | | |
| 10 mM Tris with cRNA (300 ng per mL) | (as needed for final | |
| | 30 μL volume) | |
| 10 x concentrated Accuprime Buffer #2 | 3 | μL |
| Mix of 40 modular barcoded primers | 4.8 | μL |
| (50 μM total stock) | | |
| (final ~200 nM each) | | |
| Accuprime Taq polymerase | 0.6 | μL |
| Total | 30 | μL |

Temperature Cycling Conditions (Carried Out on a BioRad iCycler®)
a. 94° C. for 120 sec
b. 94° C. for 20 sec
c. 60° C. for 20 sec (this step provides more time for annealing)
d. 55° C. for 1 min (may decrease this temperature to improve primer annealing)
e. 72° C. for 20 sec
f. repeat steps b-e for 3 more cycles (total 4 cycles)
g. 4° C. for up to 20 minutes

As quickly as possible once the reactions had reached 4° C., 1 μL of 300 mM EDTA was added (to make a final concentration of 10 mM) to terminate the activity of the polymerase. Each tube was agitated gently to ensure even mixing of the EDTA. Because the primer-extended molecules had sample-specific barcodes attached, the products of all reactions that were derived from different samples could be pooled together into a single tube.

### Purification of Primer-Extended Products

The purification of primer-extended products was achieved via pull-down and elution steps using complementary biotinylated “capture” oligonucleotides and streptavidin-agarose beads (Thermo-Fisher). First, a mixture of complementary biotinylated oligonucleotides was added to the pooled primer-extension products. These oligonucelotides were designed to anneal to the specific sequences that should be produced if the primers were extended using the intended genomic DNA target region as their templates. A list of the 40 biotinylated oligos that were used in the present example is included in Table 8. By capturing with these biotinylated oligos, it was possible to ensure that only the specifically extended primers were isolated, and that any un-extended primers and any primers that were extended on non-specific DNA templates were not pulled down. For every 30 microliter reaction volume (plus 1 microliter of EDTA added), a final concentration of 200 nM of each biotinylated oligo was added (by addition of 3.5 μL of an 80 micromolar oligonucleotide mix for a final total concentration of 8 micromolar biotinylated oligos [all 40 oligos]). Annealing of the biotinlyated capture oligos with the primer-extended products was achieved by heating the mixture to 95° C. for 30 seconds, then to 70° C. for 20 seconds, then cooling by 2.5° C. every 20 seconds until the mixtures reached 25° C.

TABLE 8
List of biotinylated target-specific capture DNA
oligonucleotides

| | | SEQ ID |
|---|---|---|
| NAME | DNA OLIGO SEQUENCE | NO: |
| BNTP53-1 | 5′-BIO-CAAGATGTTTTGCCAACTGGCC | 133 |
| BNTP53-2 | 5′-BIO-CCTGTCGTCTCTCCAGCCCCAG | 134 |
| BNTP53-3 | 5′-BIO-GGCTGGTTGCCCAGGGTCCC | 135 |
| BNTP53-4 | 5′-BIO-GCCACTGACAACCACCCTTAACC | 136 |
| BNTP53-5 | 5′-BIO-CCTCATCTTGGGCCTGTGTTATCT | 137 |
| BNTP53-6 | 5′-BIO-GGGCAGCTCGTGGTGAGGC | 138 |
| BNPIK-1 | 5′-BIO- | 139 |
| | AAGAAACAGAGAATCTCCATTTTAGCAC | |
| BNPIK-2 | 5′-BIO-TCAGTTCAATGCATGCTGTTTAATTGTG | 140 |
| BNBRAF | 5′-BIO-CTTACCATCCACAAAATGGATCCAGAC | 141 |
| BNFoxL2 | 5′-BIO-CGGAAGGGCCTCTTCATGCGGC | 142 |
| BNGNAS | 5′-BIO-GGCTTACTGGAAGTTGACTTTGTCCAC | 143 |
| BNCTNN | 5′-BIO-AGGACTTGGGAGGTATCCACATCC | 144 |
| BNPPP-1 | 5′-BIO-CTGCTGCCTCAGGATCCCCGTCC | 145 |
| BNPPP-2 | 5′-BIO-GTGGGACAATGTCAACGGTCGCT | 146 |
| BNPTEN-1 | 5′-BIO-CCCATAGAAATCTAGGGCCTCT | 147 |
| BNPTEN-2 | 5′-BIO-CTCTACTTTGATATCACCACACACAGG | 148 |
| BNKIT-1 | 5′-BIO- | 149 |
| | CTTGGCAGCCAGAAATATCCTCCTTACTC | |
| BNKIT-2 | 5′-BIO-CTCCCATTTGTGATCATAAGGAAGTTG | 150 |
| BNKIT-3 | 5′-BIO-ATACCAATCTATTGTGGGCTCTGG | 151 |
| BNEG-1 | 5′-BIO-CCCACACAGCAAAGCAGAAAC | 152 |
| BNEG-2 | 5′-BIO-AGCCACCTCCTTACTTTGCCTCC | 153 |
| BNEG-3 | 5′-BIO-GTCTTTGTGTTCCCGGACATAGTCC | 154 |
| BNAKT1 | 5′-BIO-TGAAGGTGCCATCATTCTTGAGGAG | 155 |
| BNATM | 5′-BIO-GAAGTGTCGAAGACAGCTGGTGAA | 156 |
| BNAPC | 5′-BIO-CAGCTTGCTTAGGTCCACTCTCTC | 157 |
| BNFGFR-1 | 5′-BIO-GGGCTTGTCCATGCGGTGGCC | 158 |
| BNFGFR-2 | 5′-BIO-CTCCACGTCGCTGCCCAGCACC | 159 |
| BNFGFR-3 | 5′-BIO-CAGCCGCCACCACCAGGATGAAC | 160 |
| BNMET-1 | 5′-BIO-CCTATTAAAGCAGTGCTCATGATTGG | 161 |
| BNMET-2 | 5′-BIO-CTTTCCAAAGCCATCCACTTCAC | 162 |
| BNSTK-1 | 5′-BIO-GAGTCCAGCACCTCCTTCACCTTG | 163 |
| BNSTK-2 | 5′-BIO-CGCCAGACTCACCGGGCACC | 164 |
| BNKRAS-1 | 5′-BIO- | 165 |
| | GTCACATTTTCATTATTTTTATTATAAGGCCTGC | |
| BNKRAS-2 | 5′-BIO- | 166 |
| | GTATTATTTATGGCAAATACACAAAGAAAGC | |
| BNNRAS-1 | 5′-BIO-GATGTGGCTCGCCAATTAACCCTGA | 167 |
| BNNRAS-2 | 5′-BIO-CTTGCTATTATTGATGGCAAATACACAG | 168 |
| BNHRAS-1 | 5′-BIO-GGGCAGGAGACCCTGTAGGAG | 169 |
| BNHRAS-2 | 5′-BIO-CAAAAGACTTGGTGTTGTTGATGGCA | 170 |
| BNK-Ctrl | 5′-BIO- | 171 |
| | AGAAAGGATTCTTTATGTTTCTCTTCAGG | |
| BNB-Ctrl | 5′-BIO-GAGATTCCAATAACTGTTGTTCTCTTTGT | 172 |

5′-Bio = 5′-Biotin

Then, 7 μL of high capacity streptavidin-agarose bead slurry (Thermo-Fisher) was added (per 30 μL primer-extension reaction). Tubes were turned end-over-end constantly for at least 2 hours to promote binding of biotinylated oligos to the streptavidin beads. Beads were then centrifuged briefly, and any unbound supernatant was carefully removed, avoiding aspiration of any beads. The beads were then washed in about 200 μL of 10 mM Tris pH 7.6 and 50 mM NaCl (referred to hereafter as wash buffer). Beads were suspended in wash buffer by gentle agitation, then were briefly centrifuged, and the supernatant wash buffer was removed and discarded. A second wash was performed in the same way, except that once the beads were suspended, they were incubated at 45° C. for 30 minutes while the tube was turned end-over-end (this was to promote dissociation of any DNA molecules that may have annealed non-specifically to the biotinylated capture oligos). The beads were again centrifuged briefly, and the supernatant wash buffer was removed. The captured primer-extended products were eluted from the surface of the washed beads by heat-denaturation. Since the biotin-streptavidin interaction was not substantially disrupted by heating at 95° C., only the captured primer-extended products were eluted from the beads, whereas the biotinylated capture oligos remained bound to the beads. Elution was carried out directly into the pre-amplification PCR cocktail as described below.

### Multiplexed Pre-Amplification PCR

The purified primer-extension products were eluted directly into a cocktail of buffer, nucleotides, and primers that was used to carry out the multiplexed pre-amplification reaction. The primer-extended DNA was eluted into the following cocktail:

| Molecular grade water | (enough to make 100 μL | |
|---|---|---|
| | total reaction volume) | |
| 10x Accuprime Taq PCR buffer #1 or pfx | 10 | μL |
| buffer (with dNTPs already added) | | |
| Forward primer mix for 40 amplicons | 40 | μL |
| (20 uM Fwd mix stock, 200 nM final each) | | |
| Universal reverse primer - ExtV2Rev | 10 | μL |
| (2 uM stock, 200 nM final) | | |
| Total | 100 | μL |

The beads in the pre-amplification cocktail were heated at 95° C. for 30 seconds, were quickly and gently centrifuged, and the supernatant was transferred to a clean PCR tube. When the cocktail reached room temperature, 2 L of Accuprime hotstart Taq polymerase (or 1 μL Accuprime Pfx) was added to the tube, and mixed by pipetting up and down. Then 30 μL of mineral oil was added to prevent evaporation during thermal cycling which was carried out as follows:
a. 94° C. for 2 minutes (95° C. if using Accuprime Pfx)
b. 94° C. for 20 seconds (95° C. if using Accuprime Pfx)
c. 63° C. for 30 seconds
d. 72° C. for 20 seconds
e. repeat (b) to (d) for a total of 15 cycles
f. 72° C. for 2 minutes

Then, 11 μL of 100 mM EDTA was added (10 mM final concentration) to the completed reaction to chelate divalent cations and thus terminate polymerase activity.

The forward primers used in this pre-amplification reaction were designed to hybridize to regions on the target sequences that were nested relative to the binding sites of the biotinylated capture oligonucleotides that were used in the first primer extension reaction. This nested design provided an additional level of specificity so that the desired target DNAs would be preferentially amplified. The sequences of the universal pre-amplification reverse primer (ExtV2Rev), and the 40 different nested forward primers are listed in Table 9.

TABLE 9
List of 40 forward primers and the single universal reverse
primer (ExtV2Rev) used for the pre-amplification reaction

| | | SEQ ID |
|---|---|---|
| Name | DNA Sequence | NO: |
| ExtV2REV | CGAGACGGATCAAGCAGAAGACG | 173 |
| ExF-TP53-1 | GCCAACTGGCCAAGACCTGC | 174 |
| ExF-TP53-2 | CTCCAGCCCCAGCTGCTCAC | 175 |
| ExF-TP53-3 | GTCCCCAGGCCTCTGATTCCTC | 176 |
| ExF-TP53-4 | CCTCCCAGAGACCCCAGTTGC | 177 |
| ExF-TP53-5 | TGGGCCTGTGTTATCTCCTAGGTTG | 178 |
| ExF-TP53-6 | GCAGCTCGTGGTGAGGCTCC | 179 |
| ExF-PIK3CA-1 | AGAAACAGAGAATCTCCATTTTAGCACTTACC | 180 |
| ExF-PIK3CA-2 | TTCAATGCATGCTGTTTAATTGTGTGGAAG | 181 |
| ExF-BRAF | TCCACAAAATGGATCCAGACAACTGTTC | 182 |
| ExF-FoxL2 | GGCGCCGGTAGTTGCCCTTC | 183 |
| ExF-GNAS | GGAAGTTGACTTTGTCCACCTGGAAC | 184 |
| ExF-CTNNB1 | GGAGGTATCCACATCCTCTTCCTCAG | 185 |
| ExF-PPP2R1A-1 | CGACTCCCAGGTACTTCCGGAAC | 186 |
| ExF-PPP2R1A-2 | TGTCAACGGTCGCTCATCTACCTC | 187 |
| ExF-PTEN-1 | CCATAGAAATCTAGGGCCTCTTGTGC | 188 |
| ExF-PTEN-2 | CACCACACACAGGTAACGGCTG | 189 |
| ExF-KIT-1 | GAAATATCCTCCTTACTCATGGTCGGATCA | 190 |
| ExF-KIT-2 | CCCATTTGTGATCATAAGGAAGTTGTGTTG | 191 |
| ExF-KIT-3 | GTGGGCTCTGGGAATCCTGCTG | 192 |
| ExF-EGFR-1 | CCACACAGCAAAGCAGAAACTCAC | 193 |
| ExF-EGFR-2 | ACCTCCTTACTTTGCCTCCTTCTGC | 194 |
| ExF-EGFR-3 | GTGTTCCCGGACATAGTCCAGGAG | 195 |
| ExF-AKT1 | GCCATCATTCTTGAGGAGGAAGTAGC | 196 |
| ExF-ATM | AGACAGCTGGTGAAAAATCCCTGAAC | 197 |
| ExF-APC | TGCTTAGGTCCACTCTCTCTCTTTTCAG | 198 |
| ExF-FGFR3-1 | GCGGTGGCCCTCCTTCAGCAG | 199 |
| ExF-FGFR3-2 | CCAGCACCGCCGTCTGGTTG | 200 |
| ExF-FGFR3-3 | CCACCAGGATGAACAGGAAGAAGC | 201 |
| ExF-MET-1 | CAGTGCTCATGATTGGGTCCGT | 202 |
| ExF-MET-2 | GCCATCCACTTCACTGGCAGC | 203 |
| ExF-STK11-1 | CTTCACCTTGCCGTAAGAGCCTTC | 204 |
| ExF-STK11-2 | CTCACCGGGCACCGTGAAGTC | 205 |
| ExF-KRAS-1 | CATTATTTTTATTATAAGGCCTGCTGAAAATGACTGA | 206 |
| ExF-KRAS-2 | TGGCAAATACACAAAGAAAGCCCTCC | 207 |
| ExF-NRAS-1 | CAATTAACCCTGATTACTGGTTTCCAACAG | 208 |
| ExF-NRAS-2 | GGCAAATACACAGAGGAAGCCTTCG | 209 |
| ExF-HRAS-1 | CAGGAGACCCTGTAGGAGGACC | 210 |
| ExF-HRAS-2 | TGATGGCAAACACACACAGGAAGC | 211 |
| ExF-KRAS- | AGGATTCTTTATGTTTCTCTTCAGGCAACTG | 212 |
| Cntrl | | |
| ExF-BRAF-Cntrl | ACTGTTGTTCTCTTTGTTGGAGTGCATC | 213 |

### Purification of the Products of the Pre-Amplification Reaction

The products of the pre-amplification reaction were purified using a QIAquick® PCR purification kit (Qiagen) according to the manufacturer's instructions. This removed the enzyme, dNTPs, and unincorporated primers from the double-stranded reaction products. Elution of the DNA from the column was carried out in 60 μL of EB buffer (composed of 10 mM Tris). This elution volume allowed 1 μL to be used in each of the 40 individual PCRs (see next section), with approximately 20 μL left over in case any failed reactions need to be repeated. The purified DNA can be stored at 4° C. for several days if necessary. Extra care was taken when handling any of the amplified products to avoid contamination of these products into the reagents used for reaction set-up (separate work-spaces were maintained for reagents and for amplification products).

### Separate PCR Amplification of Individual Gene Targets (Mutation Hotspots)

After purification, products of the pre-amplification reaction were subjected to further amplification by PCR in separate tubes (one tube for each of the 40 target gene regions). These individual PCRs were performed in order to provide an additional layer of amplification specificity, since the multiplexed pre-amplification reaction was likely to have produced many spurious products in addition to the amplicons of interest. Using PCR primers that were nested relative to the primers used in the previous pre-amplification step allowed the desired target DNAs to be preferentially amplified. Also, by carrying out each individual PCR to saturation and using the same concentration of primers in each reaction, similar numbers of copies of each target region could be produced. Normalization of molecular counts in this way allowed a similar sequencing depth to be achieved for each target.

A different gene-specific forward primer was paired with a universal reverse primer in each of the 40 PCR tubes. Both primers were nested relative to the primers used in the pre-amplification reaction so that further amplification specificity could be achieved (a nested primer is designed so that its 3′-end hybridizes to a region within the desired target sequence that was flanked by the primers used in the earlier round of amplification). The forward primers contained extra sequences on their 5′-ends that were necessary for subsequent sequencing on an Illumina flow cell. The reverse primer was also designed to produce a product that was compatible with the Illumina sequencer without the need for attachment of additional adapter sequences. The sequences of the universal reverse PCR primer (called IntV2Rev) and the 40 different, target-specific forward PCR primers are listed in Table 10. A 4 nucleotide stretch of degenerate sequence was included in the forward primer to provide greater sequence diversity at the first few read positions, thereby improving cluster discrimination on the Illumina sequencer. Although these primers were designed to be compatible with the Illumina next-generation sequencing system, the method can relatively easily be adapted to other sequencing platforms. The PCR setup of each individual tube was as follows:

| | | |
|---|---|---|
| Molecular grade water | 4.8 | μL |
| 10x Accuprime Taq Buffer #1 | 1 | μL |
| Forward gene-specific primer (1 uM stock, 200 nM | 2 | μL |
| final) | | |
| Universal reverse primer IntV2Rev (2 uM stock, | 1 | μL |
| 200 nM final) | | |
| Template DNA purified after pre-amplification | 1 | μL |
| reaction | | |
| Accuprime Taq DNA polymerase | 0.2 | μL |
| Total | 10 | μL |

TABLE 10
List of 40 nested forward primers and the single nested universal reverse primer
(IntV2Rev)

| | | |
|---|---|---|
| IntV2REV | CAAGCAGAAGACGGCATACGAGA | 217 |
| InF- | AATGATACGGCGACCACCGAGATCTACACTCTTTCCCTACACGACGCTCTTCCGAT | 218 |
| TP53- | CTNNNNACTTGCAGCTGTGGGTTGATTCCAC | |
| 1 | | |
| InF- | AATGATACGGCGACCACCGAGATCTACACTCTTTCCCTACACGACGCTCTTCCGAT | 219 |
| TP53- | CTNNNNACTCCAGCTGCTCACCATCGCTATCT | |
| 2 | | |
| InF- | AATGATACGGCGACCACCGAGATCTACACTCTTTCCCTACACGACGCTCTTCCGAT | 220 |
| TP53- | CTNNNNACTTCCTCACTGATTGCTCTTAGGTCTGG | |
| 3 | | |
| InF- | AATGATACGGCGACCACCGAGATCTACACTCTTTCCCTACACGACGCTCTTCCGAT | 221 |
| TP53- | CTNNNNACTCAAACCAGACCTCAGGCGGCTC | |
| 4 | | |
| InF- | AATGATACGGCGACCACCGAGATCTACACTCTTTCCCTACACGACGCTCTTCCGAT | 222 |
| TP53- | CTNNNNACTGCTCTGACTGTACCACCATCCAC | |
| 5 | | |
| InF- | AATGATACGGCGACCACCGAGATCTACACTCTTTCCCTACACGACGCTCTTCCGAT | 223 |
| TP53- | CTNNNNACTTCCCCTTTCTTGCGGAGATTCTCT | |
| 6 | | |
| InF- | AATGATACGGCGACCACCGAGATCTACACTCTTTCCCTACACGACGCTCTTCCGAT | 224 |
| PIK3CA- | CTNNNNACTCACTTACCTGTGACTCCATAGAAAATCTTTC | |
| 1 | | |
| InF- | AATGATACGGCGACCACCGAGATCTACACTCTTTCCCTACACGACGCTCTTCCGAT | 225 |
| PIK3CA- | CTNNNNACTGAAGATCCAATCCATTTTTGTTGTCCAGC | |
| 2 | | |
| InF- | AATGATACGGCGACCACCGAGATCTACACTCTTTCCCTACACGACGCTCTTCCGAT | 226 |
| BRAF | CTNNNNACTCAAACTGATGGGACCCACTCCATC | |
| InF- | AATGATACGGCGACCACCGAGATCTACACTCTTTCCCTACACGACGCTCTTCCGAT | 227 |
| FoxL2 | CTNNNNACTCGGTAGTTGCCCTTCTCGAACATG | |
| InF- | AATGATACGGCGACCACCGAGATCTACACTCTTTCCCTACACGACGCTCTTCCGAT | 228 |
| GNAS | CTNNNNACTACTTGGTCTCAAAGATTCCAGAAGTCAG | |
| InF- | AATGATACGGCGACCACCGAGATCTACACTCTTTCCCTACACGACGCTCTTCCGAT | 229 |
| CTNNB1 | CTNNNNACTCCTCAGGATTGCCTTTACCACTCAG | |
| InF- | AATGATACGGCGACCACCGAGATCTACACTCTTTCCCTACACGACGCTCTTCCGAT | 230 |
| PPP2R1A- | CTNNNNACTCCGGAACCTGTGCTCAGATGACAC | |
| 1 | | |
| InF- | AATGATACGGCGACCACCGAGATCTACACTCTTTCCCTACACGACGCTCTTCCGAT | 231 |
| PPP2R1A- | CTNNNNACTCTGTGAACTTGTCAGCCACCATGTAG | |
| 2 | | |
| InF- | AATGATACGGCGACCACCGAGATCTACACTCTTTCCCTACACGACGCTCTTCCGAT | 232 |
| PTEN- | CTNNNNACTCCTTTAAAAATTTGCCCCGATGTAATAAATATGC | |
| 1 | | |
| InF- | AATGATACGGCGACCACCGAGATCTACACTCTTTCCCTACACGACGCTCTTCCGAT | 233 |
| PTEN- | CTNNNNACTGGCTGAGGGAACTCAAAGTACATGAAC | |
| 2 | | |
| InF- | AATGATACGGCGACCACCGAGATCTACACTCTTTCCCTACACGACGCTCTTCCGAT | 234 |
| KIT-1 | CTNNNNACTGGATCACAAAGATTTGTGATTTTGGTCTAGC | |
| InF- | AATGATACGGCGACCACCGAGATCTACACTCTTTCCCTACACGACGCTCTTCCGAT | 235 |
| KIT-2 | CTNNNNACTGGTCTATGTAAACATAATTGTTTCCATTTATCT | |
| InF- | AATGATACGGCGACCACCGAGATCTACACTCTTTCCCTACACGACGCTCTTCCGAT | 236 |
| KIT-3 | CTNNNNACTGCCACACATTGGAGCATGCCA | |
| InF- | AATGATACGGCGACCACCGAGATCTACACTCTTTCCCTACACGACGCTCTTCCGAT | 237 |
| EGFR4 | CTNNNNACTGAAACTCACATCGAGGATTTCCTTGTTG | |
| InF- | AATGATACGGCGACCACCGAGATCTACACTCTTTCCCTACACGACGCTCTTCCGAT | 238 |
| EGFR- | CTNNNNACTTGCATGGTATTCTTTCTCTTCCGCAC | |
| 2 | | |
| InF- | AATGATACGGCGACCACCGAGATCTACACTCTTTCCCTACACGACGCTCTTCCGAT | 238 |
| EGFR- | CTNNNNACTGAGGCAGCCGAAGGGCATGAG | |
| 3 | | |
| InF- | AATGATACGGCGACCACCGAGATCTACACTCTTTCCCTACACGACGCTCTTCCGAT | 240 |
| AKT1 | CTNNNNACTGTGGCCGCCAGGTCTTGATG | |
| InF- | AATGATACGGCGACCACCGAGATCTACACTCTTTCCCTACACGACGCTCTTCCGAT | 241 |
| ATM | CTNNNNACTCATGTGTAGAAAGCAGATTTCTCCATGATTC | |
| IrIF- | AATGATACGGCGACCACCGAGATCTACACTCTTTCCCTACACGACGCTCTTCCGAT | 242 |
| APC | CTNNNNACTTTCAGCAGTAGGTGCTTTATTTTTAGGTAC | |
| InF- | AATGATACGGCGACCACCGAGATCTACACTCTTTCCCTACACGACGCTCTTCCGAT | 243 |
| FGFR34 | CTNNNNACTCAGCTTGAAGAGCTCCTCCACAG | |
| InF- | AATGATACGGCGACCACCGAGATCTACACTCTTTCCCTACACGACGCTCTTCCGAT | 244 |
| FGFR3- | CTNNNNACTCCTGCAGGATGGGCCGGTG | |
| 2 | | |
| InF- | AATGATACGGCGACCACCGAGATCTACACTCTTTCCCTACACGACGCTCTTCCGAT | 245 |
| FGFR3- | CTNNNNACTCCACCCCGTAGCTGAGGATGC | |
| 3 | | |
| InF- | AATGATACGGCGACCACCGAGATCTACACTCTTTCCCTACACGACGCTCTTCCGAT | 246 |
| MET- | CTNNNNACTGCTGGAGACATCTCACATTGTTTTTGTTG | |
| 1 | | |
| InF- | AATGATACGGCGACCACCGAGATCTACACTCTTTCCCTACACGACGCTCTTCCGAT | 247 |
| MET- | CTNNNNACTGCTTTGCACCTGTTTTGTTGTGTACAC | |
| 2 | | |
| InF- | AATGATACGGCGACCACCGAGATCTACACTCTTTCCCTACACGACGCTCTTCCGAT | 248 |
| STK114 | CTNNNNACTCCCATCAGGTACTTGCCGATGAG | |
| InF- | AATGATACGGCGACCACCGAGATCTACACTCTTTCCCTACACGACGCTCTTCCGAT | 249 |
| STK11- | CTNNNNACTCTGAGTGTAGATGATGTCATCCTCGATG | |
| 2 | | |
| InF- | AATGATACGGCGACCACCGAGATCTACACTCTTTCCCTACACGACGCTCTTCCGAT | 250 |
| KRAS4 | CTNNNNACTGCTGAAAATGACTGAATATAAACTTGTGGTA | |
| InF- | AATGATACGGCGACCACCGAGATCTACACTCTTTCCCTACACGACGCTCTTCCGAT | 251 |
| KRAS-2 | CTNNNNACTCAGTCCTCATGTACTGGTCCCTCATT | |
| InF- | AATGATACGGCGACCACCGAGATCTACACTCTTTCCCTACACGACGCTCTTCCGAT | 252 |
| NRAS4 | CTNNNNACTTCTTGCTGGTGTGAAATGACTGAGTAC | |
| InF- | AATGATACGGCGACCACCGAGATCTACACTCTTTCCCTACACGACGCTCTTCCGAT | 253 |
| NRAS-2 | CTNNNNACTTCGCCTGTCCTCATGTATTGGTCT | |
| InF- | AATGATACGGCGACCACCGAGATCTACACTCTTTCCCTACACGACGCTCTTCCGAT | 254 |
| HRAS-1 | CTNNNNACTCCTGAGGAGCGATGACGGAATATAAG | |
| InF- | AATGATACGGCGACCACCGAGATCTACACTCTTTCCCTACACGACGCTCTTCCGAT | 255 |
| HRAS-2 | CTNNNNACTATGTACTGGTCCCGCATGGCG | |
| InF- | AATGATACGGCGACCACCGAGATCTACACTCTTTCCCTACACGACGCTCTTCCGAT | 256 |
| KRAS- | CTNNNNACTAGGACTTTTAGAATTCTTAAATGTCATCCGC | |
| Cntrl | | 257 |
| InF- | AATGATACGGCGACCACCGAGATCTACACTCTTTCCCTACACGACGCTCTTCCGAT | |
| BRAF- | CTNNNNACTTCTAGCTTGCTGGTGTATTCTTCATAGG | |
| Cntrl | | |

Mineral oil (20 μL) was added to each tube to prevent evaporation during PCR. Again, both Accuprime Taq polymerase as well as Accuprime Pfx were tested for PCR amplification, and both worked. The temperature cycling conditions used for PCR were as follows:

a. 94° C. for 2 minutes (95° C. if using Accuprime Pfx)

b. 94° C. for 20 seconds (95° C. if using Accuprime Pfx)

c. 64° C. for 30 seconds

d. 72° C. for 20 seconds

e. repeat b to d for a total of 36 to 45 cycles (36 cycles for Taq and 45 cycles for Pfx).

f. 72° C. for 2 minutes

g. 4° C. until removed from thermal cycler
While the PCR tubes were still at 4° C., 5 μL of 30 mM EDTA was added to inactivate the polymerase (10 mM EDTA final). This was added under the mineral oil layer, and was pipetted up and down to mix. Products from all 40 reactions were pooled into a single tube (equal volumes from each of the 40 reactions were added to the final mix).

### Preparation of DNA for Next-Generation Sequencing

The pooled PCR reaction products were purified on a 2% agarose gel with ethidium bromide and 1× TBE buffer. Since all PCR products were of a similar final length, the pooled products appeared on the gel as a somewhat diffuse band. This diffuse band was excised from the gel using a fresh scalpel blade, ensuring that the gel was cut a few millimeters above and below the visible band to include any low-intensity bands that may have run faster or slower and were not well-visualized. Using a QIAquick® Gel Extraction kit (Qiagen) according to the manufacturer's instructions, the DNA was isolated from the gel slice. The DNA was eluted into 50 μL of elution buffer, EB.

### Next-Generation Sequencing

To prepare the sample for loading onto an Illumina HiSeq flow cell, the concentration of the DNA was measured using an Agilent Bioanalyzer®, and the DNA was diluted to the concentration recommended by Illumina. In order to increase sequence diversity on the flow cell, Phi-X control DNA (Illumina) was added so that the total molar amount of Phi-X DNA was approximately 30% of the final sample that was loaded onto the flow cell.

Cluster formation was carried out on the flow cell according to Illumina's protocol. The sample was loaded onto a single lane of a flow cell. The sequencing was performed on a HiSeq® 2000 instrument in multiplexed paired-end mode, with a read length of 75 base pairs in each direction. An index read was also performed, and the length of the index read was increased from the standard 7 cycles up to 13 cycles so that our longer custom barcodes and MLT sequences could be appropriately read. A control lane was designated that contained either phi-X DNA or genomic DNA so that matrix generation for phasing/prephasing would be based on a sample having greater sequence diversity than was present in our sample. Demultiplexing of the sequences was performed using custom computer code.

### Outline of Algorithm for Sequence Analysis

The sequences that survived the filtering process were comprised of the PCR amplicons of interest as well as sequences derived from control Phi-X DNA. Our algorithm effectively ignored Phi-X sequences because those sequences did not conform to the filtering requirements described below.

A computer algorithm was designed to sort, align, and count the millions of sequences that were generated by the high-throughput sequencer. The sequence elements used in the algorithm are identified in FIG. 14. The following steps provide an outline of the process and rationale used to analyze the sequence data:
1. Only clonal sequences that had passed Illumina's chastity filter were included in the analysis. Any sequences that had a “.” at any position were eliminated (these were counted as dot rejects). If an unusually large number of dots were found at a particular sequence position (indicating sequencer failure at that cycle), the filter was modified in order to avoid filtering out an unreasonably large fraction of sequences.
2. The 8 nucleotide barcode from the index read (read #2) was used to assign each filtered clonal sequence to a sample-specific bin. The sequence in the region of the barcode was expected to be in the format, BBBBBBBBNNAT, where “B” was a barcode nucleotide and “N” was a nucleotide belonging to the molecular lineage tag (a position designated as N had an approximately equal probability of being A, C, G, or T in any given molecule. In order for the clonal sequence to be assigned to a sample-specific bin, the following conditions had to be satisfied:

a. the sequence BBBBBBBB at positions 1-8 had to exactly match the reverse complement of one of the 96 barcodes listed in Table 6;

AND

b. the nucleotides at position 11 and 12 of the index read had to be AT. If a clonal sequence failed to satisfy both above conditions, it was classified as a barcode reject. In case the lack of sequence diversity at these positions 11 and 12 caused the read quality to be greatly diminished, leading to a high rate of miscalls or “.” calls, requirement (b) was optionally modified or eliminated.
3. Each clonal sequence that was assigned to a sample-specific bin was further sub-classified according to the targeted gene segment from which it arose. The primer sequences from both the forward and reverse reads were used to assign each clone to a particular gene segment. In the present example, 40 distinct gene segments were analyzed. In order to assign a clonal sequence to a gene target bin, the conditions (a), (b), and (c) had to be satisfied.

a. In the forward read, the first 8 nucleotides of the primer sequence (designated by a “F” in FIG. 14) had to exactly match the first 8 nucleotides of one of the 40 forward gene-specific primer sequences.

b. In the reverse read, the first 8 nucleotides of the primer sequence (designated by a “R” in FIG. 14) had to exactly match the first 8 nucleotides of one of the 40 reverse gene-specific primer sequences.

c. The forward primer and reverse primer reads had to lead to assignment of each clone to the same gene segment. Assignment of a single clonal sequence to more than one gene segment bin was not permissible.
If a clonal sequence failed to satisfy these conditions, it was classified as a gene segment reject.
4. Each clonal sequence that was successfully assigned to a sample-specific barcode bin and to a gene segment bin then had its forward and reverse reads aligned to each other using a Smith-Waterman algorithm, as described in Example 1 (the reverse-complement of read #3 was derived to facilitate alignment). This enabled identification of the region of overlap between the forward and reverse reads. Different lengths of overlap were expected for different gene segments since the forward and reverse read-lengths were constant but the PCR amplicon length differed for different gene segments. The length of overlap could also vary because of the presence of insertion or deletion mutations. The forward and reverse reads were also aligned to the wild-type reference sequence for its assigned gene segment (the full-length wild-type reference sequences are listed in Table 11).

TABLE 11
List of wild-type reference sequences for all 40 targeted gene segments

| Gene | | |
|---|---|---|
| Segment | Reference Sequence (FFFF....FFFF[xxxx......xxxx]RRRR.....RRRR) | |
| TP53-1 | TGCAGCTGTGGGTTGATTCCAC[acccccgcccggcacccgcgtccgcgccatggcca | 258 |
| | tcta]CAAGCAGTCACAGCACAG | |
| TP53-2 | CCAGCTGCTCACCATCGCTATCT[gagcagcgctcatggtgggggcagcgcctcac] | 259 |
| | AACCTCCGTCATGTGCTA | |
| TP53-3 | TCCTCACTGATTGCTCTTAGGTCTGG[cccctcctcagcatcttatccgagtggaagg | 260 |
| | a]AATTTGCGTGTGGAGTATTTGT | |
| TP53-4 | CAAACCAGACCTCAGGCGGCTC[atagggcaccaccacactatgtcgaa]AAGTG | 261 |
| | TTTCTGTCATCCAAATAT | |
| TP53-5 | GCTCTGACTGTACCACCATCCAC[tacaactacatgtgtaacagttcctgcatgggcggc | 262 |
| | atgaaccggaggc]CCATCCTCACCATCATCACAG | |
| TP53-6 | TCCCCTTTCTTGCGGAGATTCTCT[tcctctgtgcgccggtctcctcccaggacaggcac | 263 |
| | aaacacgcac]CTCAAAGCTGTTCCGTCCCAG | |
| PIK3CA- | CACTTACCTGTGACTCCATAGAAAATCTTTC[tcctgctcagtgatttcagag]A | 264 |
| 1 | GAGGATCTCGTGTAGAAATTGCA | |
| PIK3CA- | GAAGATCCAATCCATTTTTGTTGTCCAGC[caccatgatgtgcatcat]TCATT | 265 |
| 2 | TGTTTCATGAAATACTCCAAAGA | |
| BRAF | CAAACTGATGGGACCCACTCCATC[gagatttcactgtagc]TAGACCAAAA | 266 |
| | TCACCTATTTTTACTGTT | |
| FoxL2 | CGGTAGTTGCCCTTCTCGAACATG[tcttcgcaggccgg]GTCCAGCGTCC | 267 |
| | AGTAGTTGCA | |
| GNAS | ACTTGGTCTCAAAGATTCCAGAAGTCAG[gacacggcagcga]AGCAGGT | 268 |
| | CCTGAAACAAAATTGAG | |
| CTNNB1 | CCTCAGGATTGCCTTTACCACTCAG[agaaggagctgtggtagtggcaccagaatgg | 269 |
| | attccagagtcc]AGGTAAGACTGTTGCTGCCAG | |
| PPP2R1A- | CCGGAACCTGTGCTCAGATGACAC[ccccatggtgcggcgggcc]GCAGCCT | 270 |
| 1 | CCAAGCTGGGT | |
| PPP2R1A- | CTGTGAACTTGTCAGCCACCATGTAG[cggacgcgccaggact]TGTCTTCA | 271 |
| 2 | GCGGCCTGGCT | |
| PTEN- | CCTTTAAAAATTTGCCCCGATGTAATAAATATGC[acatatcattacaccagtt | 272 |
| 1 | cgtcc]CTTTCCAGCTTTACAGTGAATTGCC | |
| PTEN- | GGCTGAGGGAACTCAAAGTACATGAAC[ttgtcttcccgtcgtgtgg]GTCCTG | 273 |
| 2 | AATTGGAGGAATATATCTTCAT | |
| KIT-1 | GGATCACAAAGATTTGTGATTTTGGTCTAGC[cagagacatcaagaat]GAT | 274 |
| | TCTAATTATGTGGTTAAAGGAAACGC | |
| KIT-2 | GGTCTATGTAAACATAATTGTTTCCATTTATCT[cctcaacaaccttccactgta | 275 |
| | cttcatacatgggttt]CTGTGGGGAGAAAGGGAAAAC | |
| KIT-3 | GCCACACATTGGAGCATGCCA[ttcacgagcctgtcgtaagtcag]GATTTCTGG | 276 |
| | TTTTGCTACAGGAAC | |
| EGFR- | GAAACTCACATCGAGGATTTCCTTGTTG[gctttcggagatgttgcttctcttaattcc | 277 |
| 1 | ttgatagc]GACGGGAATTTTAACTTTCTCACCG | |
| EGFR- | TGCATGGTATTCTTTCTCTTCCGCAC[ccagcagtttggccagcc]CAAAATC | 278 |
| 2 | TGTGATCTTGACATGCTT | |
| EGFR- | GAGGCAGCCGAAGGGCATGAG[ctgcgtgatg]AGCTGCACGGTGGAGG | 279 |
| 3 | TGAG | |
| AKT1 | GTGGCCGCCAGGTCTTGATG[tactcccct]ACAGACGTGCGGGTGGTC | 280 |
| ATM | CATGTGTAGAAAGCAGATTTCTCCATGATTC[atttgtatcttgg]AGTAAA | 281 |
| | ATATCATGAATCAAGTATGGAAGA | |
| APC | TTCAGCAGTAGGTGCTTTATTTTTAGGTAC[ttctcgcttg]GTTTGAGCT | 282 |
| | GTTTGAGGAGGTC | |
| FGFR3- | CAGCTTGAAGAGCTCCTCCACAG[ggatgccggggtacggggagcccc]CCAGC | 283 |
| 1 | GTGAAGATCTCCCAT | |
| FGFR3- | CCTGCAGGATGGGCCGGTG[cggggagcgctctgtggg]GGCAGATGACGCT | 284 |
| 2 | CAGGGA | |
| FGFR3- | CCACCCCGTAGCTGAGGATGC[ctgcatacacactgcccgcc]TCGTCAGCCT | 285 |
| 3 | CCACCAGCG | |
| MET-1 | GCTGGAGACATCTCACATTGTTTTTGTTG[acgatcttgttgaaga]AGTCGT | 286 |
| | TGACATATTTGATAGGGAAC | |
| MET-2 | GCTTTGCACCTGTTTTGTTGTGTACAC[tatagtattctttatcataca]TGTCTCT | 287 |
| | GGCAAGACCAAAATT | |
| STK11- | CCCATCAGGTACTTGCCGATGAG[cttggcccgcttgcggcgcggctggtaga]TG | 288 |
| 1 | ACCTCGGTGGAGTCGATA | |
| STK11- | CTGAGTGTAGATGATGTCATCCTCGATG[tcgaagaggtcctcgtcctcgtccgcg | 289 |
| 2 | c]CGTGCAGGTCCTCCAAGTAT | |
| KRAS- | GCTGAAAATGACTGAATATAAACTTGTGGTA[gttggagctggtggcgt]AG | 290 |
| 1 | GCAAGAGTGCCTTGACGAC | |
| KRAS- | CAGTCCTCATGTACTGGTCCCTCATT[gcactgtactcctcttg]ACCTGCTGT | 291 |
| 2 | GTCGAGAATATCG | |
| NRAS- | TCTTGCTGGTGTGAAATGACTGAGTAC[aaactggtggtggttggagcaggtggtg | 292 |
| 1 | t]TGGGAAAAGCGCACTGAT | |
| NRAS- | TCGCCTGTCCTCATGTATTGGTCT[ctcatggcactgtactcttcttg]TCCAGCTG | 293 |
| 2 | TATCCAGTATGTCA | |
| HRAS- | CCTGAGGAGCGATGACGGAATATAAG[ctggtggtggtgggcgccggcggtgt]G | 294 |
| 1 | GGCAAGAGTGCGCTGACCAC | |
| HRAS- | ATGTACTGGTCCCGCATGGCG[ctgtactcctcctg]GCCGGCGGTATCCAG | 295 |
| 2 | GATGA | |
| KRAS- | AGGACTTTTAGAATTCTTAAATGTCATCCGC[at]AGGTGTTTTGTCA | 296 |
| Cntrl | ATATTATAAACAGGAT | |
| BRAF- | TCTAGCTTGCTGGTGTATTCTTCATAGG[cctataaaataaagcagacttatat]TC | 297 |
| Cntrl | AATCCGGACTTTGTCCTGAT | |

Note:
Forward and reverse primer sequences are in capital letters, to the left and right of the square brackets, respectively. The actual reverse primer sequence would be the reverse-complement of that shown above. The genomic wild-type amplicon target sequence is in lower case letters within the square brackets.

5. Next, any variants or mutations that existed within the amplicon target region for each gene segment were identified and quantified (nucleotides in this region were designated by a “X” in FIG. 14). Wild-type sequences of the amplicon target regions (region between flanking primers) for all 40 gene segments are listed in lower case letters within square brackets in Table 11. All clones belonging to a particular sample-specific bin and a particular gene segment bin were compared to the wild-type sequence in the amplicon target region. If a clonal sequence had perfect agreement between its two overlapping reads in the amplicon target region, but deviated from the wild-type sequence, then that clone was identified and counted as a “consistent variant”. If a clonal sequence had perfect agreement between its two overlapping reads in the amplicon target region, and was perfectly consistent with the wild-type sequence, then it was identified and counted as an “exact match to wild type”. If a clonal sequence had deviations from the amplicon target reference sequence seen in either or both of the forward and reverse reads, but the two reads were not perfectly consistent with each other, then that clone was identified and counted as an “inconsistent variant”. Any mismatches, insertions, or deletions relative to the reference sequence (whether found in both reads or in a single read) were counted and tabulated for each position within the amplicon target region for all sequences in a given bin (For purposes of illustration, results of a hypothetical experiment are shown in FIG. 15).
6. In order to distinguish mutant sequences that were present in the original template DNA molecules from those arising due to sequencing errors or errors introduced during PCR amplification or sample processing, sequences called “molecular lineage tags” (MLTs) were used. As shown in FIG. 14, the sequence for MLT-1 was comprised of a total of 8 degenerate nucleotide positions (derived by concatenating 6 positions of MLT-la and 2 positions of MLT-1b). Each of the eight N positions had an approximately equal likelihood of having an A, C, G, or T nucleotide, so that 4\hat{ }8 =65,536 possible MLT sequences could be generated. Thus, a particular primer molecule would be expected to have any one of the 65,536 possible MLT sequences. Prior to amplification by PCR, the DNA template molecules were copied by primer-extension, and a MLT-1 sequence became attached to each primer-extended copy. Thus, each template copy was tagged with one of 65,536 possible MLT-1 sequences.

To identify variants arising from mutant template DNA molecules, first a list of all “consistent variants” was generated. If a “consistent variant” sequence was seen in more than one clone within a bin of sequences, then the number of copies of such variants was counted. These variants were listed along with the number of clonal copies (in descending order of frequency) as shown in FIG. 15. Then, for all clones belonging to a particular “consistent variant” sequence, a list of MLT-1 sequences associated with the clones was generated (the actual list of MLT-1 sequences was not displayed). Within each list, any MLT sequence that was found to be associated with more than one clone was classified as a “multiply occurring MLT”. A histogram of such multiply occurring MLTs was generated for each variant (as shown in FIG. 15). The count of different MLT-1 sequences occurring “N” times for a given variant was listed in a numerical table (where N was the number of copies of the same MLT). An alternate way to present the MLT-1 counts was to list the “N” value and the number of different MLTs having that number of copies (e.g. N×Z, where Z is the number of different MLT sequences having N copies). For variant sequences arising from one or more mutant DNA template molecules, there was a high probability of finding multiply occurring MLTs with a high “N” value (because the number of clonal sequences sampled post-amplification was several-fold greater than the number of template DNA molecules that were copied and tagged in the primer-extension reaction). In contrast, for variant sequences arising from errors introduced during amplification of wild-type template molecules, it was unlikely to find MLT-1 sequences with “N” values as high as those associated with true mutant templates. Since known mutant template oligonucleotides had been spiked into each sample prior to amplification, these internal standards were used to determine the range of “N” values that should be expected for variant sequences derived from unknown mutant templates. Values falling below that range were presumed to be associated with variants arising from errors of amplification or sequencing.
7. A “mutation authenticity score” (MAS) can be used to facilitate the identification of variant sequences arising from mutant template DNA molecules. The MLT copy numbers, “N”, that are associated with the spiked-in mutant internal control oligos (having mutations at two distinct positions) can be evaluated (Table 7). The variant sequences that arise from these authentic mutant templates are associated with MLT-1 sequences having relatively high “N” values. The value “Nₐᵤₜₕ” is in one embodiment the mean “N” value for these known authentic mutant templates. The “Nₐᵤₜₕ” value can be weighted or unweighted. If a mutation were introduced during the first cycle of PCR, the “N” value of such a variant sequence would be approximately (1/1.7)×Nₐᵤₜₕ (if each cycle of PCR yields approximately 1.7-fold amplification). Similarly, a variant sequence would have “N” values of approximately (1/1.7y)×Nₐᵤₜₕ (if a mutation were introduced during the y^{th} cycle of PCR). Thus, a mutation authenticity score is calculated for each “consistent variant” sequence based on how close the “N” values of its MLTs are to the “Nₐᵤₜₕ” values of the authentic mutants that are spiked into the reaction. A variant would be likely to be authentic if its “N” values were distributed within a defined range of the Nₐᵤₜₕ values.

### Results

A set of control plasma-derived DNA samples was tested. These samples contained various ratios of normal plasma DNA spiked with known amounts of mutant oligonucleotides (listed in Table 7). It was consistently observed that the PCR products were formed in a highly specific manner for all 40 gene segments included in the panel. The methods were extensively tested using a real-time quantitative thermal cycler, and comparisons to negative controls having no plasma DNA or having mouse DNA confirmed that the intended targets were being amplified. The products of all 40 PCRs were run on an agarose gel, and the production of appropriate-sized amplicons was confirmed.

Sequencing of the 40 pooled amplicons from multiple barcoded samples on the Illumina HiSeq® 2000 platform further confirmed that all intended gene segments were amplified. The total number of raw clonal sequences yielded was 282,965,036. After filtering, the rejected sequences were as follows:

| | |
|---|---|
| Failed Illumina's chastity filter: | 79,320,290 |
| Positions 5-7 in forward read were not “ACT”: | 23,751,168 |
| Rejected because of the presence of an N in | 27,477,576 |
| position 8 or beyond: | |
| Failed to recognize forward primer: | 6,304,833 |
| Barcode did not exactly match one in our set | 15,921,744 |
| of 96: | |
| Positions 11-12 of index read were not “AT”: | 2,903,342 |
| Failed to recognize reverse primer: | 17,064,651 |
| Remaining filtered reads: | 110,221,432 |

The total number of filtered counts assigned to each of the 40 gene segments is listed in Table 12. These data revealed a relatively even distribution of counts across the various amplicons.

TABLE 12
Number of filtered sequence counts associated
with each targeted gene segment.

| | |
|---|---|
| AKT1 | 1927236 |
| APC | 3263261 |
| ATM | 2988621 |
| BRAF | 2644671 |
| BRAF-Cntrl | 2827920 |
| CTNNB1 | 3387874 |
| EGFR-1 | 2582553 |
| EGFR-2 | 2670482 |
| EGFR-3 | 1908549 |
| FGFR3-1 | 2441848 |
| FGFR3-2 | 1907661 |
| FGFR3-3 | 2154173 |
| FoxL2 | 2782971 |
| GNAS | 2481154 |
| HRAS-1 | 2173717 |
| HRAS-2 | 2456244 |
| KIT-1 | 1960170 |
| KIT-2 | 4202032 |
| KIT-3 | 2739896 |
| KRAS-1 | 5647782 |
| KRAS-2 | 3421539 |
| KRAS-Cntrl | 3076757 |
| MET-1 | 2923088 |
| MET-2 | 2956664 |
| NRAS-1 | 4037334 |
| NRAS-2 | 2462906 |
| PIK3CA-1 | 2124016 |
| PIK3CA-2 | 2807504 |
| PPP2R1A-1 | 2087213 |
| PPP2R1A-2 | 1966662 |
| PTEN-1 | 3125952 |
| PTEN-2 | 2562118 |
| STK11-1 | 2969119 |
| STK11-2 | 2613351 |
| TP53-1 | 2494793 |
| TP53-2 | 2248788 |
| TP53-3 | 3074251 |
| TP53-4 | 2964973 |
| TP53-5 | 2632869 |
| TP53-6 | 2522720 |
| Total | 110221432 |

The sequence data were processed using a modified version of the computer code that was used in Example 1. The results demonstrated that control double-mutant oligonucelotides that were spiked into plasma DNA could be reliably detected and quantified. Requiring consistency of overlapping paired-end reads appeared to eliminate the vast majority sequencer errors. Also, analysis of the MLT sequences associated with “consistent variants” made it possible to distinguish sequences arising from authentic mutant templates from those introduced during amplification or sequencing. An example of processed data for the BRAF gene target region for a sample in which approximately equal numbers of copies of normal plasma DNA and double-mutant control oligos were mixed is shown in FIG. 16. This output represented the analysis of data from a single bin (single gene segment, single barcode). In this example, a total count of 103,742 clonal sequences were assigned to the bin, and 65,143 of these counts arose from amplification of the double-mutant oligonucleotide that was spiked in. The double-mutant sequences comprised approximately half of the total sequences in this bin. The MLT counts associated with each consistent variant were listed in the format N×y where y was the number of unique MLT sequences that had N copies associated with that particular consistent variant. It was observed that the variant sequence arising from spiked-in control mutant templates was associated with several MLT-1 sequences having high copy numbers (N). The highest value of N for this variant was 3742, and there were many distinct MLTs that had copy numbers in the thousands. In contrast, the next most abundant variant had only 965 total counts and was associated with only a few distinct MLTs. The highest value of N for this variant was 576, and only one other MLT had a count in the hundreds range. All other consistent variants in the list were associated with very low MLT copy numbers. Based on these observations, it could be confirmed that only the spiked-in control oligonucleotides produced variant sequences that had high MLT counts. MLT counts associated with variant sequences that likely arose from errors of PCR or sequencing were much lower, as predicted.

### Example 3

This example demonstrates the application of methods that incorporated methods of Example 2, and included modifications thereof. A modification included elimination of separate PCRs for each target DNA in the final step. Instead, the final amplification was performed in a single tube using universal PCR primers. This also eliminated the requirement for a pre-amplification step. Pooled amplification was made possible by copying, tagging, and purifying the targeted DNA regions in a highly selective manner; spurious templates that could be amplified by universal primers in the final PCR would be minimized (FIG. 1). In this example, the same 40 genomic target regions were analyzed as in Example 2.

### Methods

### Preparation of Mixtures of Primers Having Combinations of Modular Oligonucleotide Segments

Mixtures of primers having combinations of modular barcode segments and gene-specific segments were prepared as described in Example 2. The preferred approach, called “modular automated synthesis and purification”, is schematized in FIG. 3, and is described in detail in Example 2.

### Collection and Processing of Patient Plasma Samples

Blood was collected and processed as described in Example 2.

### Extraction and Purification of DNA From Plasma

DNA was extracted from plasma as described in Example 2.

### Round 1 PCR

In order to make a limited number of tagged copies (fewer than 20) of the plasma-derived template DNA molecules, a few cycles of PCR were performed (in contrast to primer extension that was performed in Example 2). The reverse primers used in the first round of PCR were the modular barcoded mixtures of gene-specific primers as described above (same as the primers used in the primer extension reaction in Example 2). For forward primers, the same oligonucleotides were used as the biotinylated capture oligonucleotides that had been used in Example 2 to purify the primer-extension products. The sequences of the forward primers are listed in Table 8.

Forty different gene regions were targeted, and therefore a combination of 40 different biotinylated forward primers and 40 different modular barcoded gene-specific reverse primers were used in the Round 1 PCR for each sample. For a given sample, the mixture of gene-specific reverse primers all had the same, sample-specific barcode in the 5′ segment. The primer mixes were produced so that an approximately equimolar concentration of 40 different forward and 40 different reverse primers would be present in the reaction (final concentration of approximately 100 nM each primer). In addition to sample-specific barcodes, the reverse primers also contained degenerate sequence regions known as molecular lineage tags (MLTs) as well as common sequences at the 5′-end that allowed for hybridization of “universal” PCR primers in subsequent steps. The MLT assigned to each copy in Round 1 PCR was referred to as MLT-1.

Control DNA molecules containing known mutations were spiked into each Round 1 PCR to serve as internal quantitative standards. As described in Example 2, these DNA molecules were cartridge-purified oligonucleotides that were synthesized to contain variations from the wild-type sequence at two distinct positions. These variations allowed the control sequences to be readily distinguished from other variants within DNA purified from a clinical sample. The sequences of the top strands of these control DNA oligonucleotides are listed in Table 7. Bottom strands were also synthesized corresponding to the reverse complements of these 40 sequences. In order to make the control DNA as similar as possible to the clinically-derived DNA, both strands were annealed to make them double-stranded before adding them to the primer-extension reaction. The double-stranded DNA was quantified by UV spectrometry and then diluted to the desired concentration. To each PCR, approximately 200 copies of the double-stranded control DNA fragments corresponding to each of the 40 gene target sites were added.

The Round 1 PCR amplification consisted of the following components: (1) template DNA purified from plasma and eluted in 20 microliters of Qiagen elution buffer AVE, (2) 1× Phusion® buffer HF, (3) 200 mM of each dNTP (dATP, dCTP, dGTP, and dTTP), (4) mixture of 40 reverse barcoded primers, 100 nM each, (5) mixture of 40 forward biotinylated primers, 100 nM each, (6) 200 copies of double-stranded control DNA, (7) molecular grade water as needed to make the desired total volume, and (8) Phusion® Hot Start Flex DNA polymerase, (0.04 U/μL). The total volume of each reaction was 40 microliters (for each 20 μ eluted plasma DNA sample). A separate reaction was set up for each sample.

Thermal cycling was carried out on a BioRad iCycler® using the following protocol: (1) 98° C. for 45 seconds, (2) 98° C. for 10 seconds, (3) 70° C. for 30 seconds, (4) slowly cooling by 1° C. every 30 seconds down to 56° C., (5) 55° C. for 2 minutes, (6) 72° C. for 1 minute, (7) repeat steps 2 to 6 for 3 cycles total, and (8) hold temperature at 72° C. indefinitely.

As quickly as possible, while the reaction was still at 72° C., EDTA (10 mM final concentration) was added to terminate the polymerase activity. Each tube was agitated gently to ensure even mixing of the EDTA. Since the PCR products now had sample-specific barcodes attached, the products of all reactions could be pooled together into a single tube.

### Purification of Round 1 PCR Products

Since the forward primers used in the Round 1 PCR contained biotin tags at their 5′-ends, these tags were incorporated into the PCR products and were used to purify the products. To capture the biotin-tagged PCR products, 10 μL of high capacity streptavidin-agarose bead slurry (Thermo-Fisher) was added (per 40 μL PCR). Thus, for example, if fifty Round 1 PCRs were performed in a volume of 40 μL each, then the volume after combining all samples would be 2 mL, and 500 μL of bead slurry would be used. Tubes were turned end-over-end constantly for at least 2 hours at room temperature to promote binding of biotinylated DNA to the streptavidin beads. Beads were then gently and briefly centrifuged at low speed, and any unbound supernatant was carefully removed, avoiding aspiration of any beads. The beads were then washed in 200 μL of buffer containing 10 mM Tris pH 7.6, 50 mM NaCl, and 1 mg/mL salmon sperm DNA (“wash buffer”). Beads were suspended in wash buffer by gentle agitation, were gently centrifuged, and then the supernatant wash buffer was discarded. A second wash was performed in the same way, except that the suspended beads were incubated at 50° C. for 25 minutes followed by 60° C. for 5 minutes while the tube was turned end-over-end to promote dissociation of any DNA molecules that may have annealed non-specifically to the biotinylated oligonucleotides. The beads were again centrifuged gently, and the supernatant wash buffer was removed.

Optionally, between the first and second washes, the beads were treated with Exonuclease I (New England Biolabs) in order to digest any single stranded DNA (including un-extended biotinylated primer) that was bound to the beads. For the tested samples, it was found that this nuclease treatment was not necessary following the first Round of PCR. For digestion, the beads were suspended in 1× Exonuclease I buffer (2 μL for every 1 μL of beads), and then Exonuclease

I enzyme was added to a final concentration of 0.5 μL. The reaction was incubated at 37° C. for 30 minutes. The beads were then centrifuged, the supernatant was discarded, and the beads then were subjected to the second wash.

The captured PCR products were then eluted from the surface of the washed beads by heat-denaturation. Elution was carried out by heating the beads to 95° C. for 30 seconds directly in Round 2 PCR cocktail (as described below), gently centrifuging the beads, and harvesting the eluted DNA within the supernatant cocktail. Note that only one strand of the PCR product was eluted because the biotin-streptavidin interaction was not substantially disrupted by heating at 95° C., and thus the biotinylated strand would remain bound to the beads. Likewise, any un-extended biotinylated oligonucleotides would also remain bound to the beads.

### Round 2 PCR

The second round of PCR was also performed for only a few cycles (between 2 and 4). This PCR provided additional selectivity by using a mixture of 40 nested forward primers that would specifically hybridize to the desired genomic target sequences. This step also provided a second molecular lineage tag on the other side of the mutation-prone target sequence (opposite to the barcode and MLT-1). The forward primers contained a stretch of degenerate positions, called “molecular lineage tag-2” (MLT-2), which was useful in determining which sequences had become labeled with the wrong barcode due to sequence crossover during pooled amplification. The forward primers also contained a common sequence at their 5′-ends which served as a universal primer binding site in the third and final round of PCR. This common sequence also provided some of the adapter sequences required for sequencing on the Illumina platform. The reverse primer used in Round 2 PCR had a biotin tag at its 5′-end which was used for purification of the Round 2 PCR products.

The purified Round 1 PCR products were eluted directly into a cocktail that was used for Round 2 PCR. For every 10 μL of bead slurry that was used, 40 μL of PCR cocktail was used for elution. The Round 2 PCR cocktail consisted of the following components: (1) 1× Phusion® buffer HF, (2) 200 mM of each dNTP (dATP, dCTP, dGTP, and dTTP), (3) a mixture of 40 nested forward primers, 100 nM each, (4) 10 ng/μL salmon sperm DNA, and (5) molecular grade water as needed to make the desired total volume.

After elution of the single-stranded PCR product from the beads into the above cocktail (and removal of the beads), a biotinylated universal reverse primer was added to achieve a final concentration of 200 nM. This biotinylated primer had to be added to the cocktail after removal of the streptavidin-agarose beads to prevent the biotin from binding to the beads. Finally, Phusion® Hot Start Flex DNA polymerase was added to the cocktail to a final concentration of 0.04 units per microliter, and was mixed by gently pipetting the cocktail up and down. If the total volume was greater than recommended for a single PCR tube, then the cocktail was split into the appropriate number of identical reaction volumes.

Thermal cycling was carried out on a BioRad iCycler® using the following protocol: (1) 98° C. for 45 seconds, (2) 98° C. for 10 seconds, (3) 70° C. for 30 seconds, (4) slowly cooling by 1° C. every 30 seconds down to 61° C., (5) 60° C. for 2 minutes, (6) 72° C. for 1 minute, (7) repeat steps 2 to 6 for 3 cycles total and, (8) hold temperature at 72° C. indefinitely.

As quickly as possible, while the reaction was still at 72° C., EDTA (10 mM final concentration) was added to terminate the polymerase activity. The tube was agitated gently to ensure even mixing of the EDTA.

The sequences of the 40 nested forward primers were the same as those provided in Table 10, except that the sequence “NNNNACT” in each primer was replaced by “NNNNNN”. The common “ACT” sequence was removed because it led to poor sequence diversity which produced low-quality base-calls on the Illumina sequencer. Instead, the stretch of degenerate positions was increased from 4 to 6 bases to provide a greater number of sequence combinations at MLT-2. The sequence of the biotinylated reverse primer used in Round 2 PCR (called BioV2rev) was as follows: 5′-Biotin-CGAGACGGATCAAGCA GAAGACG-3′ (SEQ ID NO:214).

### Purification of Round 2 PCR Products

The biotin tag at the 5′-end of the reverse primer used in Round 2 PCR was used to capture and purify the products of Round 2 PCR. This step removed any un-extended forward primers, as well as many spurious products that might have been produced during the amplification, which prevented inappropriate incorporation of new MLTs during the next round of amplification.

The capture, washing, digestion, and elution of the Round 2 PCR products was performed in a manner that was essentially identical to the process described above for the purification of Round 1 PCR products. In Round 2 PCR purification, the Exonuclease I step was not optional. Thus, the beads were washed once in wash buffer at room temperature, then were treated with Exonuclease I, and then were washed a second time at elevated temperature (50° C. for 25 minutes followed by 60° C. for 5 minutes) to remove non-specific DNA. Fewer beads were used for a given volume of Round 2 PCR reaction. Five microliters of bead slurry was used for every 40 μL of PCR reaction volume.

Elution of the captured PCR products was also performed in a manner that was essentially the same as that used for purification of the Round 1 PCR products. The streptavidin-agarose beads were heated to 95° C. for 30 seconds to elute the product directly into a cocktail that was used for Round 3 PCR (described below). The biotinylated strand of the PCR product remained bound to the beads, while the opposite strand was eluted into the Round 3 PCR cocktail.

### Round 3 PCR

The third and final round of PCR amplified the DNA molecules that were specifically tagged, copied, and purified in the first 2 rounds of PCR. To provide sufficient DNA for visualization by ethidium bromide staining on an agarose gel, the amount of PCR product from Round 3 had to be substantial (at least 0.5 microgram). Thus, the final PCR amplification was carried to saturation or beyond (typically 15 to 35 cycles, depending on the amount of template DNA in each sample and the total number of samples that were pooled).

In contrast to the final PCRs in Example 2 which were performed separately for each genomic target site, the final PCR in the present Example was performed in a combined reaction volume for all genomic targets and for all samples. This extremely high level of multiplexing was only possible because of the highly selective methods used for amplification and purification in the prior two rounds of PCR.

As described above, the round 2 PCR products were eluted directly into Round 3 PCR cocktail. The volume of this cocktail depended on the volume of beads used. For every 5 μL of bead slurry, 20 μL of PCR cocktail was used. The Round 3 PCR cocktail consisted of the following components: (1) 1× Phusion® buffer HF, (2) 200 mM of each dNTP (dATP, dCTP, dGTP, and dTTP), (3) Universal forward and reverse primers, 200 nM each, (4) 10 ng/μL salmon sperm DNA, and (5) molecular grade water as needed to make the desired total volume.

After elution of the single-stranded PCR product from the beads into the above cocktail, Phusion® Hot Start Flex DNA polymerase was added to the cocktail to a final concentration of 0.04 U/μL, and was mixed by gently pipetting the cocktail up and down. If the total volume was greater than recommended for a single PCR tube, then the cocktail was split into the appropriate number of identical reaction volumes. Mineral oil (20 μL) was added to the tube(s) to prevent evaporation during PCR.

Thermal cycling was carried out on a BioRad® iCycler using the following protocol: (1) 98° C. for 45 seconds, (2) 98° C. for 10 seconds, (3) 62° C. for 30 seconds, (4) 72° C. for 20 seconds, (5) repeat steps 2 to 4 for 35 cycles total, and (8) hold temperature at 4° C. indefinitely.

Soon after the reaction had reached 4° C., EDTA (10 mM final concentration) was added to terminate the polymerase activity. Since the PCR product was under mineral oil, a pipette with a filtered tip was used to evenly mix the EDTA. Special care was taken to avoid contamination of other reagents and workspaces with PCR products.

### Preparation of DNA for Next-Generation Sequencing

The product of the Round 3 PCR was purified on a 2% agarose gel, as described in Example 2. Since the products were not of a homogeneous length, a somewhat diffuse band was seen on the gel. The band was cut with a few mm margin above and below to ensure inclusion of any low-intensity bands that may have been difficult to visualize. A QIAquick® Gel Extraction kit (Qiagen) was used to isolate the DNA from the gel slice. The DNA was eluted into 50 μL of EB buffer (supplied in the kit).

### Next-Generation Sequencing

Next generation sequencing was performed as described in Example 2, using the Illumina HiSeq® 2000 platform. In the present example, the Illumina MiSeq® instrument was also used with similar success for samples requiring less sequence depth. In contrast to Example 2, addition of Phi-X DNA to improve sequence diversity was not necessary in the present Example because modification of the Round 2 PCR forward primers to remove the common “ACT” sequence and to lengthen MLT-2 resulted in adequate sequence diversity.

### Outline of Algorithm for Sequence Analysis

Essentially the same algorithm that was described in Example 2 was applied to the data generated in Example 3. Although many of the processing steps used in Example 3 differ from those used in Example 2, the structure of the final double stranded DNA products are virtually identical. Thus, a very similar algorithm can be applied for sorting, aligning, and counting the resulting sequences. As noted above, the region of MLT-2 which was “NNNNACT” in Example 2 was replaced with “NNNNNN” in Example 3, and this change was accounted for in the modified algorithm.

To minimize the probability of mis-classifying a variant sequence as belonging to the wrong sample, MLT-1 and MLT-2 sequences were used to distinguish sequences in which barcode “cross-over” may have occurred during pooled amplification. Since a portion of MLT-1 is adjacent to the barcode sequence, and MLT-2 is on the other side of the target region (FIG. 14), molecules that undergo such cross-over between the barcode and the mutation-prone region would also undergo cross-over between MLT-1 and MLT-2 (or between the two separate regions of MLT-1). Such “crossed-over” sequences would be expected to have a low number of copies having a given combination of MLT-1 and MLT-2 sequences. In contrast, sequences arising from an authentic mutant template that remained attached to its originally assigned barcode would be expected to have greater copies of a given MLT-1 and MLT-2 combination.

The algorithm in Example 3 was modified to facilitate evaluation of the relationship between MLT-1 and MLT-2 sequence counts for each “consistent variant” and also for the wild-type sequences. In order to report these counts in a reasonably succinct format, it was necessary to bin MLT counts by powers of two. For example, an MLT-1 count of 13 would be placed into bin 4 (because 2\hat{ }4 is the smallest power of 2 that is greater than or equal to 13). Thus, a report of 4×5 meant that there were five instances of counts in the range of 9 to 16. Similarly, a report of 3×6 meant that there were six instances of counts in the range of 5 to 8. For a given collection of MLT-1 counts, the associated MLT-2 counts were reported in a similar format, to the right of the MLT-1 counts and separated by colons. For example, 4×5:2×3:1×7 meant that among 5 sets of MLT-1 sequences occurring between 9 and 16 times, there were 3 instances of MLT-2 sequences that occurred between 3 and 4 times, and 7 instances of MLT-2 sequences that occurred twice. Different MLT-1 bins were separated by a space.

### Results

Purified DNA that was obtained from 0.5 mL of plasma of healthy volunteers was mixed with various amounts of the control mutant oligonucleotides listed in Table 7. Between 200 and 5,000 copies of each of the 40 control oligonucleotides were added to each purified plasma DNA sample. These mixtures were subjected to 3 rounds of PCR and purification as described in the methods. The highly multiplexed Round 3 PCR in which multiple gene targets from multiple samples were amplified in a single tube, resulted in the specific production of amplicons of the expected size. As shown in FIG. 17, a relatively broad band was seen migrating at a size corresponding to between 200 and 300 base pairs on a 2% non-denaturing agarose gel (approximately centered at 250 base pairs). The primers and target regions were of variable length, and the amplification products spanned a range of sizes. Extensive testing of negative controls confirmed that these products were specific and were absent when mouse DNA or no template DNA was substituted for human plasma DNA in Round 1 PCR.

The gel-extracted PCR products were subjected to next-generation sequencing using an Illumina MiSeq® instrument. The total number of raw clonal paired-end sequences was 20,511,389. After application of the various filters described above, the remaining sequences numbered 11,184,975. The 40 different gene target regions were fairly evenly represented among the filtered sequences. The median sequence count for the 40 gene-specific regions (all barcodes) was 166,867.

After processing of the sequence data using the computer algorithm described above, control mutant oligonucleotides that were spiked into the plasma DNA were identified and quantified. Importantly, they were readily distinguished from the vast majority of errors introduced during amplification, processing, or sequencing. As observed previously in Example 1 and Example 2, the sequence redundancy provided by the clonal overlapped paired-end reads was able to virtually eliminate sequencer-generated errors in the mutation-prone sequence regions. The “consistent variants” were then analyzed for the distribution of their associated MLT sequences. As an example, the summary output for analysis of sequences belonging to a single barcode and target gene region KRAS-2 (region surrounding codon 61 of the KRAS gene) is shown in FIG. 18. In this sample, approximately 200 copies of the mutant oligonucleotides were spiked in (each having two distinct mutations relative to the wild-type sequence). The mismatches of the “consistent variants” relative to the reference wild-type sequence are displayed in the lower portion of FIG. 18. This single data bin contained 13,315 total sequences, of which 10,815 were exact matches to the wild-type sequence and 1,767 were exact matches to the spiked-in mutant sequence (an exact match requires that the overlapping portions of the paired-reads agree with each other). The spiked-in mutant sequences comprised approximately 10% to 15% of the total DNA in the sample, which is in the expected range (there should be approximately 1,000 to 2,000genome copies of fragmented DNA in 0.5 mL of plasma). The counts of MLT-1 and MLT-2 are reported for each “consistent variant” according to the scheme described above. The MLT-1counts associated with sequences arising from the spiked-in control mutant oligonucleotides were generally higher than those associated with other variant sequences, as expected. This made it possible to distinguish many of the “consistent variants” arising from polymerase misincorporations that might have otherwise been mistaken for sequences arising from true mutant template molecules. Analysis of MLT-2 counts associated each group of MLT-1 counts provided insight into the efficiency of molecular tagging and copying at PCR Rounds 1 and 2. It also helped to distinguishing variants assigned to the wrong sample due to barcode cross-over during pooled amplification.

### Example 4: Splint-Mediated Enzymatic Ligation of Modular Oligonucleotide Segments

In this example, an alternative approach is described for the production of mixtures of primers in which each mixture had a common 5′ barcode segment and a variety of gene-specific 3′ segments. Enzymatic ligation was used to concentrate modular oligonucleotide segments. More specifically, in each ligation, a uniquely barcoded 5′ oligonucleotide segment was ligated to a uniform mixture of different gene-specific 3′ segments. A DNA splint was used to faciliate the ligation.

Gene-specific oligonucleotides with a common sequence at the 5′-end (and a 5′-phosphate group added during oligonucleotide synthesis) were mixed in equimolar ratios. The uniform mixture was divided into separate tubes and was ligated to a uniquely barcoded oligonucleotide in each tube using a biotin-tagged DNA splint as illustrated in FIG. 13. The sequences of the 5′-phosphorylated gene-specific oligonucleotides are listed in Table 13.

TABLE 13
List of chemically 5′-phosphorylated gene-specific oligonucleotides used
for splint-mediated modular ligation.

| Name | DNA Sequence | |
|---|---|---|
| Ph-TP53-1 | X-AGACGTGTGCTCTTCCGATCTGTGCTGTGACTGCTTG | 298 |
| Ph-TP53-2 | X-AGACGTGTGCTCTTCCGATCTAGCACATGACGGAGGTT | 299 |
| Ph-TP53-3 | X-AGACGTGTGCTCTTCCGATCTCAAATACTCCACACGCAAATT | 300 |
| Ph-TP53-4 | X-AGACGTGTGCTCTTCCGATCTATTTGGATGACAGAAACACTT | 301 |
| Ph-TP53-5 | X-AGACGTGTGCTCTTCCGATCTTGTGATGATGGTGAGGATGG | 302 |
| Ph-TP53-6 | X-AGACGTGTGCTCTTCCGATCTGGGACGGAACAGCTTTGAG | 303 |
| Ph-PIK3CA-1 | X-AGACGTGTGCTCTTCCGATCTGCAATTTCTACACGAGATCCTCT | 304 |
| Ph-PIK3CA-2 | X-AGACGTGTGCTCTTCCGATCTCTTTGGAGTATTTCATGAAACAAATGA | 305 |
| Ph-BRAF | X-AGACGTGTGCTCTTCCGATCTACAGTAAAAATAGGTGATTTTGGTCTA | 306 |
| Ph-FoxL2 | X-AGACGTGTGCTCTTCCGATCTGCAACTACTGGACGCTGGAC | 307 |
| Ph-GNAS | X-AGACGTGTGCTCTTCCGATCTCAATTTTGTTTCAGGACCTGCT | 308 |
| Ph-CTNNB1 | X-AGACGTGTGCTCTTCCGATCTGGCAGCAACAGTCTTACCT | 309 |
| Ph-PPP2R1A- | X-AGACGTGTGCTCTTCCGATCTCCCAGCTTGGAGGCTGC | 310 |
| 1 | | |
| Ph-PPP2R1A- | X-AGACGTGTGCTCTTCCGATCTGCCAGGCCGCTGAAGACA | 311 |
| 2 | | |
| Ph-PTEN-1 | X-AGACGTGTGCTCTTCCGATCTGCAATTCACTGTAAAGCTGGAAAG | 312 |
| Ph-PTEN-2 | X-AGACGTGTGCTCTTCCGATCTGAAGATATATTCCTCCAATTCAGGAC | 313 |
| Ph-KIT-1 | X- | 314 |
| | AGACGTGTGCTCTTCCGATCTCGTTTCCTTTAACCACATAATTAGAATC | |
| Ph-KIT-2 | X-AGACGTGTGCTCTTCCGATCTTTTCCCTTTCTCCCCACAG | 315 |
| Ph-KIT-3 | X-AGACGTGTGCTCTTCCGATCTTCCTGTAGCAAAACCAGAAATC | 316 |
| Ph-EGFR-1 | X-AGACGTGTGCTCTTCCGATCTGGTGAGAAAGTTAAAATTCCCGTC | 317 |
| Ph-EGFR-2 | X-AGACGTGTGCTCTTCCGATCTAGCATGTCAAGATCACAGATTTTG | 318 |
| Ph-EGFR-3 | X-AGACGTGTGCTCTTCCGATCTCACCTCCACCGTGCAGCT | 319 |
| Ph-AKT1 | X-AGACGTGTGCTCTTCCGATCTACCACCCGCACGTCTGT | 320 |
| Ph-ATM | X- | 321 |
| | AGACGTGTGCTCTTCCGATCTCTTCCATACTTGATTCATGATATTTTACT | |
| Ph-APC | X-AGACGTGTGCTCTTCCGATCTACCTCCTCAAACAGCTCAAAC | 322 |
| Ph-FGFR3-1 | X-AGACGTGTGCTCTTCCGATCTTGGGAGATCTTCACGCTGG | 323 |
| Ph-FGFR3-2 | X-AGACGTGTGCTCTTCCGATCTCCCTGAGCGTCATCTGCC | 324 |
| Ph-FGFR3-3 | X-AGACGTGTGCTCTTCCGATCTGCTGGTGGAGGCTGACGA | 325 |
| Ph-MET-1 | X-AGACGTGTGCTCTTCCGATCTTCCCTATCAAATATGTCAACGACT | 326 |
| Ph-MET-2 | X-AGACGTGTGCTCTTCCGATCTATTTTGGTCTTGCCAGAGACA | 327 |
| Ph-STK11-1 | X-AGACGTGTGCTCTTCCGATCTATCGACTCCACCGAGGTCA | 328 |
| Ph-STK11-2 | X-AGACGTGTGCTCTTCCGATCTACTTGGAGGACCTGCACG | 329 |
| Ph-KRAS-1 | X-AGACGTGTGCTCTTCCGATCTCGTCAAGGCACTCTTGCCT | 330 |
| Ph-KRAS-2 | X-AGACGTGTGCTCTTCCGATCTGATATTCTCGACACAGCAGGT | 331 |
| Ph-NRAS-1 | X-AGACGTGTGCTCTTCCGATCTTCAGTGCGCTTTTCCCA | 332 |
| Ph-NRAS-2 | X-AGACGTGTGCTCTTCCGATCTGACATACTGGATACAGCTGGA | 333 |
| Ph-HRAS-1 | X-AGACGTGTGCTCTTCCGATCTGGTCAGCGCACTCTTGCCC | 334 |
| Ph-HRAS-2 | X-AGACGTGTGCTCTTCCGATCTCATCCTGGATACCGCCGGC | 335 |
| Ph-KRAS-C | X-AGACGTGTGCTCTTCCGATCTCCTGTTTATAATATTGACAAAACACCT | 336 |
| Ph-BRAF-C | X-AGACGTGTGCTCTTCCGATCTCAGGACAAAGTCCGGATTGA | 337 |

X = 5′-posphate added chemically during oligonucleotide synthesis.

The sequences of the 96 different barcoded oligonucleotides contained the following common sequence, with each oligonucleotide containing a different 8-nucleotide barcode from the list in Table 6 inserted into the position marked [BC1-96]:

(SEQ ID NO: 215)
5′-CGAGACGGATCAAGCAGAAGACGGCATACGAGAT[BC1-
96]NNNNNNGTGACTGGAGTTC-3′

The sequence of the 3′-biotin tagged splint oligonucleotide was:

(SEQ ID NO: 216)
5′-ATCGGAAGAGCACACGTCTGAACTCCAGTCACAAAAAAAAAAAAATC
TCGTATGCCGTCTTCTGCTTGATCCG TCTCG-3′-Biotin-TEG

The barcoded oligonucleotides were cartridge purified to ensure that they were mostly full-length. They were synthesized at the 40 nmole scale, with an expected full-length yield of approximately 50 to 60%. The phosphorylated gene-specific oligonucleotides and splint oligonucleotide were purified on a polyacrylamide gel (as described in Sambrook J J, et al., Molecular Cloning: A Laboratory Manual, Cold Spring Harbor Press, 2001).
The ligation reactions were carried out using the following conditions:

| | | |
|---|---|---|
| Molecular grade water | 38.15 | μL |
| Dithiothreitol (100 mM stock) | 2.5 | μL |
| NEB Ligase buffer (10x) | 10 | μL |
| 5′-Phosphorylated oligo mix (263 uM stock) | 2.7 | μL |
| (7 uM final) | | |
| splint oligo (248 uM stock) (14 uM final) | 5.65 | μL |
| Barcoded oligo (50 uM stock) (20 uM final) | 40 | μL |
| Total | 100 | μL |

The 5′-phosphorylated oligonucleotide mix consisted of an equimolar mixture of 40 different gene-specific oligos. 96 different reactions were set up in separate tubes, each one with a different barcoded oligonucleotide. To anneal the oligonucleotides to the splint, the reaction mixes were heated on a thermal cycler as follows: 95° C. for 30 sec, 70° C. for 20 sec, then the temperature was decreased by 2.5° C. every 20 sec until the samples reached 25° C.

Then 2 μL of T4 DNA Ligase (400,000 U/mL, New England Biolabs) was added to each reaction, and after mixing, the reactions were incubated at 25° C. for at least 2 hours.

Then 20 μL of streptavidin-agarose high-capacity bead slurry (Thermo Scientific, Pierce) was added, and the samples were incubated at room temperature while being turned end-over-end on a rotisserie for at least 2 hrs.

The streptavidin-agarose beads were then washed three times with 200 microliters of Tris 10 mM pH 7.6, NaCl 50 mM. The ligated (and unligated) DNA molecules were then eluted from the beads by heat-denaturation of the DNA duplex. The majority of the biotinylated splint oligo remained attached to the beads because the biotin-steptavidin interaction was not significantly disrupted by heating at 95° C. The elution was carried out in 2 steps. In the first step, the beads were heated to 95° C. in 40 microliters of the Tris/NaCl buffer for 30 seconds. The beads were quickly spun down by brief centrifugation, and then the supernatant containing was removed and stored. In the second step, the same elution process was carried out, but with heating to 95° C. for 45 seconds in order to remove any remaining ligated DNA from the beads. The supernatants containing the ligated (and unligated) DNA from the first and second elution steps were combined into a total volume of 80 microliters. This process yielded approximately 600 to 700 picomoles of ligated oligonucleotides in 80 microliters of buffer, for a final concentration of approximately 7-8 micromolar.

1. A method of synthesizing modular oligonucleotide primer mixes, the method comprising:
a) synthesizing a plurality of 3′ oligonucleotide segments comprising a plurality of target-specific primer sequences, wherein each target-specific primer sequence is synthesized in a separate synthesis column on solid supports;
b) pausing the synthesis;
c) pooling and thoroughly mixing all solid supports from all synthesis columns containing the partially-synthesized 3′ oligonucleotide segments;
d) dispensing the pooled mixture of partially-synthesized 3′ oligonucleotide segments into a plurality of new synthesis columns;
e) resuming synthesis to add a 5′ oligonucleotide segment comprising a unique sample-specific barcode to each new synthesis column; and
f) cleaving and deprotecting the oligonucleotides from the solid supports in each column, yielding a plurality of modular oligonucleotide mixes, wherein each mix contains a unique, sample-specific barcode and a plurality of target-specific primer sequences.

2. The method of claim 1, further comprising incorporation of a molecular lineage tag in the 5′ oligonucleotide segment.

3. The method of claim 1, wherein the modular oligonucleotide primer mixes enable early assignment of sample-specific barcodes to a plurality of target sequences from a plurality of samples.

4. The method of claim 3, wherein the target sequences comprise DNA.

5. The method of claim 3, wherein the target sequences comprise RNA.

6. The method of claim 3, wherein the plurality of samples comprise clinical specimens.

7. The method of claim 6, wherein the clinical specimens are from different clinical patients.

8. The method of claim 6, wherein the clinical specimens are from different clinical time points.


SEQUENCE LISTING (370 sequences)

SEQ ID NO: 1 - DNA, 28 bp, synthetic construct
  misc_feature 1..28: note = primer
  source 1..28: mol_type = other DNA
ggcctgctgaaaatgactgaatataaac

SEQ ID NO: 2 - DNA, 28 bp, synthetic construct
  misc_feature 1..28: note = primer
  source 1..28: mol_type = other DNA
ttcgtccacaaaatgattctgaattagc

SEQ ID NO: 3 - DNA, 29 bp, synthetic construct
  misc_feature 1..29: note = primer
  source 1..29: mol_type = other DNA
tcatgaagacctcacagtaaaaataggtg

SEQ ID NO: 4 - DNA, 26 bp, synthetic construct
  misc_feature 1..26: note = primer
  source 1..26: mol_type = other DNA
cacaaaatggatccagacaactgttc

SEQ ID NO: 5 - DNA, 24 bp, synthetic construct
  misc_feature 1..24: note = primer
  source 1..24: mol_type = other DNA
gtactggtgaaaacaccgcagcat

SEQ ID NO: 6 - DNA, 28 bp, synthetic construct
  misc_feature 1..28: note = primer
  source 1..28: mol_type = other DNA
cttactttgcctccttctgcatggtatt

SEQ ID NO: 7 - DNA, 43 bp, synthetic construct
  misc_feature 1..43: note = primer
  misc_difference 3..8: note = the sequence nnnnnn can be any one of aacctt, aacgta, aagcat, aaggaa, atccat, atcgaa, atgcaa, atggta, taccta, tacgaa, tacgtt, tagctt, taggat, ttcgat, ttgcat, or ttgcta
  source 1..43: mol_type = other DNA
ggnnnnnncgaacagtctccgaatataaacttgtggtagttgg

SEQ ID NO: 8 - DNA, 41 bp, synthetic construct
  misc_feature 1..41: note = primer
  misc_difference 3..8: note = the sequence nnnnnn can be any one of aatcaa, aatgat, aagata, aacatt, atcata, atagtt, atcaat, atgtat, taacat, tagtaa, tatgta, ttacaa, ttcatt, ttagta, ttctaa, or tacaat
  source 1..41: mol_type = other DNA
gcnnnnnnggatgagtgcagtgaattagctgtatcgtcaag

SEQ ID NO: 9 - DNA, 42 bp, synthetic construct
  misc_feature 1..42: note = primer
  misc_difference 3..8: note = the sequence nnnnnn can be any one of aacctt, aacgta, aagcat, aaggaa, atccat, atcgaa, atgcaa, atggta, taccta, tacgaa, tacgtt, tagctt, taggat, ttcgat, ttgcat, or ttgcta
  source 1..42: mol_type = other DNA
ggnnnnnncgaacagtctccaaataggtgattttggtctagc

SEQ ID NO: 10 - DNA, 41 bp, synthetic construct
  misc_feature 1..41: note = primer
  misc_difference 3..8: note = the sequence nnnnnn can be any one of aatcaa, aatgat, aagata, aacatt, atcata, atagtt, atcaat, atgtat, taacat, tagtaa, tatgta, ttacaa, ttcatt, ttagta, ttctaa, or tacaat
  source 1..41: mol_type = other DNA
gcnnnnnnggatgagtgcagccagacaactgttcaaactga

SEQ ID NO: 11 - DNA, 42 bp, synthetic construct
  misc_feature 1..42: note = primer
  misc_difference 3..8: note = the sequence nnnnnn can be any one of aacctt, aacgta, aagcat, aaggaa, atccat, atcgaa, atgcaa, atggta, taccta, tacgaa, tacgtt, tagctt, taggat, ttcgat, ttgcat, or ttgcta
  source 1..42: mol_type = other DNA
ggnnnnnncgaacagtctcccagcatgtcaagatcacagatt

SEQ ID NO: 12 - DNA, 41 bp, synthetic construct
  misc_feature 1..41: note = primer
  misc_difference 3..8: note = the sequence nnnnnn can be any one of aatcaa, aatgat, aagata, aacatt, atcata, atagtt, atcaat, atgtat, taacat, tagtaa, tatgta, ttacaa, ttcatt, ttagta, ttctaa, or tacaat
  source 1..41: mol_type = other DNA
gcnnnnnnggatgagtgcaggcatggtattctttctcttcc

SEQ ID NO: 13 - DNA, 35 bp, synthetic construct
  misc_feature 1..35: note = synthetic construct
  source 1..35: mol_type = other DNA
ccaactaccacaagtttatattcggagactgttcg

SEQ ID NO: 14 - DNA, 34 bp, synthetic construct
  misc_feature 1..34: note = synthetic construct
  source 1..34: mol_type = other DNA
aatctgtgatcttgacatgctgggagactgttcg

SEQ ID NO: 15 - DNA, 34 bp, synthetic construct
  misc_feature 1..34: note = synthetic construct
  source 1..34: mol_type = other DNA
gctagaccaaaatcacctatttggagactgttcg

SEQ ID NO: 16 - DNA, 20 bp, synthetic construct
  misc_feature 1..20: note = synthetic construct
  source 1..20: mol_type = other DNA
ggaaccttcgaacagtctcc

SEQ ID NO: 17 - DNA, 20 bp, synthetic construct
  misc_feature 1..20: note = synthetic construct
  source 1..20: mol_type = other DNA
ggaacgtacgaacagtctcc

SEQ ID NO: 18 - DNA, 20 bp, synthetic construct
  misc_feature 1..20: note = synthetic construct
  source 1..20: mol_type = other DNA
ggaagcatcgaacagtctcc

SEQ ID NO: 19 - DNA, 20 bp, synthetic construct
  misc_feature 1..20: note = synthetic construct
  source 1..20: mol_type = other DNA
ggaaggaacgaacagtctcc

SEQ ID NO: 20 - DNA, 20 bp, synthetic construct
  misc_feature 1..20: note = synthetic construct
  source 1..20: mol_type = other DNA
ggatccatcgaacagtctcc

SEQ ID NO: 21 - DNA, 20 bp, synthetic construct
  misc_feature 1..20: note = synthetic construct
  source 1..20: mol_type = other DNA
ggatcgaacgaacagtctcc

SEQ ID NO: 22 - DNA, 20 bp, synthetic construct
  misc_feature 1..20: note = synthetic construct
  source 1..20: mol_type = other DNA
ggatgcaacgaacagtctcc

SEQ ID NO: 23 - DNA, 20 bp, synthetic construct
  misc_feature 1..20: note = synthetic construct
  source 1..20: mol_type = other DNA
ggatggtacgaacagtctcc

SEQ ID NO: 24 - DNA, 20 bp, synthetic construct
  misc_feature 1..20: note = synthetic construct
  source 1..20: mol_type = other DNA
ggtacctacgaacagtctcc

SEQ ID NO: 25 - DNA, 20 bp, synthetic construct
  misc_feature 1..20: note = synthetic construct
  source 1..20: mol_type = other DNA
ggtacgaacgaacagtctcc

SEQ ID NO: 26 - DNA, 20 bp, synthetic construct
  misc_feature 1..20: note = synthetic construct
  source 1..20: mol_type = other DNA
ggtacgttcgaacagtctcc

SEQ ID NO: 27 - DNA, 20 bp, synthetic construct
  misc_feature 1..20: note = synthetic construct
  source 1..20: mol_type = other DNA
ggtagcttcgaacagtctcc

SEQ ID NO: 28 - DNA, 20 bp, synthetic construct
  misc_feature 1..20: note = synthetic construct
  source 1..20: mol_type = other DNA
ggtaggatcgaacagtctcc

SEQ ID NO: 29 - DNA, 20 bp, synthetic construct
  misc_feature 1..20: note = synthetic construct
  source 1..20: mol_type = other DNA
ggttcgatcgaacagtctcc

SEQ ID NO: 30 - DNA, 20 bp, synthetic construct
  misc_feature 1..20: note = synthetic construct
  source 1..20: mol_type = other DNA
ggttgcatcgaacagtctcc

SEQ ID NO: 31 - DNA, 20 bp, synthetic construct
  misc_feature 1..20: note = synthetic construct
  source 1..20: mol_type = other DNA
ggttgctacgaacagtctcc

SEQ ID NO: 32 - DNA, 33 bp, synthetic construct
  misc_feature 1..33: note = synthetic construct
  source 1..33: mol_type = other DNA
cttgacgatacagctaattcactgcactcatcc

SEQ ID NO: 33 - DNA, 33 bp, synthetic construct
  misc_feature 1..33: note = synthetic construct
  source 1..33: mol_type = other DNA
ggaagagaaagaataccatgcctgcactcatcc

SEQ ID NO: 34 - DNA, 33 bp, synthetic construct
  misc_feature 1..33: note = synthetic construct
  source 1..33: mol_type = other DNA
tcagtttgaacagttgtctggctgcactcatcc

SEQ ID NO: 35 - DNA, 20 bp, synthetic construct
  misc_feature 1..20: note = synthetic construct
  source 1..20: mol_type = other DNA
gcaatcaaggatgagtgcag

SEQ ID NO: 36 - DNA, 20 bp, synthetic construct
  misc_feature 1..20: note = synthetic construct
  source 1..20: mol_type = other DNA
gcaatgatggatgagtgcag

SEQ ID NO: 37 - DNA, 20 bp, synthetic construct
  misc_feature 1..20: note = synthetic construct
  source 1..20: mol_type = other DNA
gcaagataggatgagtgcag

SEQ ID NO: 38 - DNA, 20 bp, synthetic construct
  misc_feature 1..20: note = synthetic construct
  source 1..20: mol_type = other DNA
gcaacattggatgagtgcag

SEQ ID NO: 39 - DNA, 20 bp, synthetic construct
  misc_feature 1..20: note = synthetic construct
  source 1..20: mol_type = other DNA
gcatcataggatgagtgcag

SEQ ID NO: 40 - DNA, 20 bp, synthetic construct
  misc_feature 1..20: note = synthetic construct
  source 1..20: mol_type = other DNA
gcatagttggatgagtgcag

SEQ ID NO: 41 - DNA, 20 bp, synthetic construct
  misc_feature 1..20: note = synthetic construct
  source 1..20: mol_type = other DNA
gcatcaatggatgagtgcag

SEQ ID NO: 42 - DNA, 20 bp, synthetic construct
  misc_feature 1..20: note = synthetic construct
  source 1..20: mol_type = other DNA
gcatgtatggatgagtgcag

SEQ ID NO: 43 - DNA, 20 bp, synthetic construct
  misc_feature 1..20: note = synthetic construct
  source 1..20: mol_type = other DNA
gctaacatggatgagtgcag

SEQ ID NO: 44 - DNA, 20 bp, synthetic construct
  misc_feature 1..20: note = synthetic construct
  source 1..20: mol_type = other DNA
gctagtaaggatgagtgcag

SEQ ID NO: 45 - DNA, 20 bp, synthetic construct
  misc_feature 1..20: note = synthetic construct
  source 1..20: mol_type = other DNA
gctatgtaggatgagtgcag

SEQ ID NO: 46 - DNA, 20 bp, synthetic construct
  misc_feature 1..20: note = synthetic construct
  source 1..20: mol_type = other DNA
gcttacaaggatgagtgcag

SEQ ID NO: 47 - DNA, 20 bp, synthetic construct
  misc_feature 1..20: note = synthetic construct
  source 1..20: mol_type = other DNA
gcttcattggatgagtgcag

SEQ ID NO: 48 - DNA, 20 bp, synthetic construct
  misc_feature 1..20: note = synthetic construct
  source 1..20: mol_type = other DNA
gcttagtaggatgagtgcag

SEQ ID NO: 49 - DNA, 20 bp, synthetic construct
  misc_feature 1..20: note = synthetic construct
  source 1..20: mol_type = other DNA
gcttctaaggatgagtgcag

SEQ ID NO: 50 - DNA, 20 bp, synthetic construct
  misc_feature 1..20: note = synthetic construct
  source 1..20: mol_type = other DNA
gctacaatggatgagtgcag

SEQ ID NO: 51 - DNA, 109 bp, synthetic construct
  misc_feature 1..109: note = synthetic construct
  source 1..109: mol_type = other DNA
aaggcctgctgaaaatgactgaatataaacttgtggtagatggagctggtggcgtaagcaagagtgccttgacgatacagctaattcagaatcattttgtggacgaata

SEQ ID NO: 52 - DNA, 45 bp, synthetic construct
  misc_feature 1..45: note = synthetic construct
  misc_difference 22..27: note = n is a, c, g, or t
  source 1..45: mol_type = other DNA
agacgtgtgctcttccgatctnnnnnnctgtgctgtgactgcttg

SEQ ID NO: 53 - DNA, 45 bp, synthetic construct
  misc_feature 1..45: note = synthetic construct
  misc_difference 22..27: note = n is a, c, g, or t
  source 1..45: mol_type = other DNA
agacgtgtgctcttccgatctnnnnnntagcacatgacggaggtt

SEQ ID NO: 54 - DNA, 49 bp, synthetic construct
  misc_feature 1..49: note = synthetic construct
  misc_difference 22..27: note = n is a, c, g, or t
  source 1..49: mol_type = other DNA
agacgtgtgctcttccgatctnnnnnnacaaatactccacacgcaaatt

SEQ ID NO: 55 - DNA, 50 bp, synthetic construct
  misc_feature 1..50: note = synthetic construct
  misc_difference 22..27: note = n is a, c, g, or t
  source 1..50: mol_type = other DNA
agacgtgtgctcttccgatctnnnnnnatatttggatgacagaaacactt

SEQ ID NO: 56 - DNA, 48 bp, synthetic construct
  misc_feature 1..48: note = synthetic construct
  misc_difference 22..27: note = n is a, c, g, or t
  source 1..48: mol_type = other DNA
agacgtgtgctcttccgatctnnnnnnctgtgatgatggtgaggatgg

SEQ ID NO: 57 - DNA, 48 bp, synthetic construct
  misc_feature 1..48: note = synthetic construct
  misc_difference 22..27: note = n is a, c, g, or t
  source 1..48: mol_type = other DNA
agacgtgtgctcttccgatctnnnnnnctgggacggaacagctttgag

SEQ ID NO: 58 - DNA, 51 bp, synthetic construct
  misc_feature 1..51: note = synthetic construct
  misc_difference 22..27: note = n is a, c, g, or t
  source 1..51: mol_type = other DNA
agacgtgtgctcttccgatctnnnnnntgcaatttctacacgagatcctct

SEQ ID NO: 59 - DNA, 55 bp, synthetic construct
  misc_feature 1..55: note = synthetic construct
  misc_difference 22..27: note = n is a, c, g, or t
  source 1..55: mol_type = other DNA
agacgtgtgctcttccgatctnnnnnntctttggagtatttcatgaaacaaatga

SEQ ID NO: 60 - DNA, 55 bp, synthetic construct
  misc_feature 1..55: note = synthetic construct
  misc_difference 22..27: note = n is a, c, g, or t
  source 1..55: mol_type = other DNA
agacgtgtgctcttccgatctnnnnnnaacagtaaaaataggtgattttggtcta

SEQ ID NO: 61 - DNA, 48 bp, synthetic construct
  misc_feature 1..48: note = synthetic construct
  misc_difference 22..27: note = n is a, c, g, or t
  source 1..48: mol_type = other DNA
agacgtgtgctcttccgatctnnnnnntgcaactactggacgctggac

SEQ ID NO: 62 - DNA, 51 bp, synthetic construct
  misc_feature 1..51: note = synthetic construct
  misc_difference 22..27: note = n is a, c, g, or t
  source 1..51: mol_type = other DNA
agacgtgtgctcttccgatctnnnnnnctcaattttgtttcaggacctgct

SEQ ID NO: 63 - DNA, 48 bp, synthetic construct
  misc_feature 1..48: note = synthetic construct
  misc_difference 22..27: note = n is a, c, g, or t
  source 1..48: mol_type = other DNA
agacgtgtgctcttccgatctnnnnnnctggcagcaacagtcttacct

SEQ ID NO: 64 - DNA, 45 bp, synthetic construct
  misc_feature 1..45: note = synthetic construct
  misc_difference 22..27: note = n is a, c, g, or t
  source 1..45: mol_type = other DNA
agacgtgtgctcttccgatctnnnnnnacccagcttggaggctgc

SEQ ID NO: 65 - DNA, 46 bp, synthetic construct
  misc_feature 1..46: note = synthetic construct
  misc_difference 22..27: note = n is a, c, g, or t
  source 1..46: mol_type = other DNA
agacgtgtgctcttccgatctnnnnnnagccaggccgctgaagaca

SEQ ID NO: 66 - DNA, 52 bp, synthetic construct
  misc_feature 1..52: note = synthetic construct
  misc_difference 22..27: note = n is a, c, g, or t
  source 1..52: mol_type = other DNA
agacgtgtgctcttccgatctnnnnnnggcaattcactgtaaagctggaaag

SEQ ID NO: 67 - DNA, 55 bp, synthetic construct
  misc_feature 1..55: note = synthetic construct
  misc_difference 22..27: note = n is a, c, g, or t
  source 1..55: mol_type = other DNA
agacgtgtgctcttccgatctnnnnnnatgaagatatattcctccaattcaggac

SEQ ID NO: 68 - DNA, 56 bp, synthetic construct
  misc_feature 1..56: note = synthetic construct
  misc_difference 22..27: note = n is a, c, g, or t
  source 1..56: mol_type = other DNA
agacgtgtgctcttccgatctnnnnnngcgtttcctttaaccacataattagaatc

SEQ ID NO: 69 - DNA, 48 bp, synthetic construct
  misc_feature 1..48: note = synthetic construct
  misc_difference 22..27: note = n is a, c, g, or t
  source 1..48: mol_type = other DNA
agacgtgtgctcttccgatctnnnnnngttttccctttctccccacag

SEQ ID NO: 70 - DNA, 51 bp, synthetic construct
  misc_feature 1..51: note = synthetic construct
  misc_difference 22..27: note = n is a, c, g, or t
  source 1..51: mol_type = other DNA
agacgtgtgctcttccgatctnnnnnngttcctgtagcaaaaccagaaatc

SEQ ID NO: 71 - DNA, 52 bp, synthetic construct
  misc_feature 1..52: note = synthetic construct
  misc_difference 22..27: note = n is a, c, g, or t
  source 1..52: mol_type = other DNA
agacgtgtgctcttccgatctnnnnnncggtgagaaagttaaaattcccgtc

SEQ ID NO: 72 - DNA, 52 bp, synthetic construct
  misc_feature 1..52: note = synthetic construct
  misc_difference 22..27: note = n is a, c, g, or t
  source 1..52: mol_type = other DNA
agacgtgtgctcttccgatctnnnnnnaagcatgtcaagatcacagattttg

SEQ ID NO: 73 - DNA, 47 bp, synthetic construct
  misc_feature 1..47: note = synthetic construct
  misc_difference 22..27: note = n is a, c, g, or t
  source 1..47: mol_type = other DNA
agacgtgtgctcttccgatctnnnnnnctcacctccaccgtgcagct

SEQ ID NO: 74 - DNA, 45 bp, synthetic construct
  misc_feature 1..45: note = synthetic construct
  misc_difference 22..27: note = n is a, c, g, or t
  source 1..45: mol_type = other DNA
agacgtgtgctcttccgatctnnnnnngaccacccgcacgtctgt

SEQ ID NO: 75 - DNA, 57 bp, synthetic construct
  misc_feature 1..57: note = synthetic construct
  misc_difference 22..27: note = n is a, c, g, or t
  source 1..57: mol_type = other DNA
agacgtgtgctcttccgatctnnnnnntcttccatacttgattcatgatattttact

SEQ ID NO: 76 - DNA, 49 bp, synthetic construct
  misc_feature 1..49: note = synthetic construct
  misc_difference 22..27: note = n is a, c, g, or t
  source 1..49: mol_type = other DNA
agacgtgtgctcttccgatctnnnnnngacctcctcaaacagctcaaac

SEQ ID NO: 77 - DNA, 47 bp, synthetic construct
  misc_feature 1..47: note = synthetic construct
  misc_difference 22..27: note = n is a, c, g, or t
  source 1..47: mol_type = other DNA
agacgtgtgctcttccgatctnnnnnnatgggagatcttcacgctgg

SEQ ID NO: 78 - DNA, 46 bp, synthetic construct
  misc_feature 1..46: note = synthetic construct
  misc_difference 22..27: note = n is a, c, g, or t
  source 1..46: mol_type = other DNA
agacgtgtgctcttccgatctnnnnnntccctgagcgtcatctgcc

SEQ ID NO: 79 - DNA, 46 bp, synthetic construct
  misc_feature 1..46: note = synthetic construct
  misc_difference 22..27: note = n is a, c, g, or t
  source 1..46: mol_type = other DNA
agacgtgtgctcttccgatctnnnnnncgctggtggaggctgacga

SEQ ID NO: 80 - DNA, 53 bp, synthetic construct
  misc_feature 1..53: note = synthetic construct
  misc_difference 22..27: note = n is a, c, g, or t
  source 1..53: mol_type = other DNA
agacgtgtgctcttccgatctnnnnnngttccctatcaaatatgtcaacgact

SEQ ID NO: 81 - DNA, 49 bp, synthetic construct
  misc_feature 1..49: note = synthetic construct
  misc_difference 22..27: note = n is a, c, g, or t
  source 1..49: mol_type = other DNA
agacgtgtgctcttccgatctnnnnnnaattttggtcttgccagagaca

SEQ ID NO: 82 - DNA, 47 bp, synthetic construct
  misc_feature 1..47: note = synthetic construct
  misc_difference 22..27: note = n is a, c, g, or t
  source 1..47: mol_type = other DNA
agacgtgtgctcttccgatctnnnnnntatcgactccaccgaggtca

SEQ ID NO: 83 - DNA, 47 bp, synthetic construct
  misc_feature 1..47: note = synthetic construct
  misc_difference 22..27: note = n is a, c, g, or t
  source 1..47: mol_type = other DNA
agacgtgtgctcttccgatctnnnnnnatacttggaggacctgcacg

SEQ ID NO: 84 - DNA, 48 bp, synthetic construct
  misc_feature 1..48: note = synthetic construct
  misc_difference 22..27: note = n is a, c, g, or t
  source 1..48: mol_type = other DNA
agacgtgtgctcttccgatctnnnnnngtcgtcaaggcactcttgcct

SEQ ID NO: 85 - DNA, 49 bp, synthetic construct
  misc_feature 1..49: note = synthetic construct
  misc_difference 22..27: note = n is a, c, g, or t
  source 1..49: mol_type = other DNA
agacgtgtgctcttccgatctnnnnnncgatattctcgacacagcaggt

SEQ ID NO: 86 - DNA, 45 bp, synthetic construct
  misc_feature 1..45: note = synthetic construct
  misc_difference 22..27: note = n is a, c, g, or t
  source 1..45: mol_type = other DNA
agacgtgtgctcttccgatctnnnnnnatcagtgcgcttttccca

SEQ ID NO: 87 - DNA, 49 bp, synthetic construct
  misc_feature 1..49: note = synthetic construct
  misc_difference 22..27: note = n is a, c, g, or t
  source 1..49: mol_type = other DNA
agacgtgtgctcttccgatctnnnnnntgacatactggatacagctgga

SEQ ID NO: 88 - DNA, 48 bp, synthetic construct
  misc_feature 1..48: note = synthetic construct
  misc_difference 22..27: note = n is a, c, g, or t
  source 1..48: mol_type = other DNA
agacgtgtgctcttccgatctnnnnnngtggtcagcgcactcttgccc

SEQ ID NO: 89 - DNA, 47 bp, synthetic construct
  misc_feature 1..47: note = synthetic construct
  misc_difference 22..27: note = n is a, c, g, or t
  source 1..47: mol_type = other DNA
agacgtgtgctcttccgatctnnnnnntcatcctggataccgccggc

SEQ ID NO: 90 - DNA, 56 bp, synthetic construct
  misc_feature 1..56: note = synthetic construct
  misc_difference 22..27: note = n is a, c, g, or t
  source 1..56: mol_type = other DNA
agacgtgtgctcttccgatctnnnnnnatcctgtttataatattgacaaaacacct

SEQ ID NO: 91 - DNA, 49 bp, synthetic construct
  misc_feature 1..49: note = synthetic construct
  misc_difference 22..27: note = n is a, c, g, or t
  source 1..49: mol_type = other DNA
agacgtgtgctcttccgatctnnnnnnatcaggacaaagtccggattga

SEQ ID NO: 92 - DNA, 57 bp, synthetic construct
  misc_feature 1..57: note = synthetic construct
  misc_difference 35..36: note = n is a, c, g, or t
  misc_difference 37..44: note = this stretch nnnnnnnn can be any one of ccgatatt , gccatatt, tctggatt, actcgatt, tcacgatt, actgcatt, tcagcatt, tctccatt, attggtgt, ttaggtgt, atacgtgt, atagctgt, attcctgt, ttacctgt, ctctatgt, ctcttagt, ctgatagt, gtcatagt, ataggagt, attcgagt, ttacgagt, attgcagt, ttagcagt, ataccagt, agtggtct, tgaggtct, tgtcgtct, tgtgctct, agtcctct, tgacctct, cgctatct, cgcttact, cggatact, ggcatact, tgtggact, agtcgact, tgacgact, agtgcact, tgagcact, tgtccact, tcattgtg, tctatgtg, actatctg, acttactg, tcatactg, attatcgg, ttatacgg, tgattgcg, tgtatgcg, agtatccg, agttaccg, tgataccg, actatgtc, acttagtc, tcatagtc, tcattctc, tctatctc, attatggc, ttataggc, ttattcgc, agtatgcc, agttagcc, tgatagcc, tctggtta, tcacgtta, tcagctta, tctcctta, ccgtatta, gcctatta, ccgttata, gccttata, tcaggata, tctcgata, tctgcata, tcaccata, ctgattga, gtcattga, ttacgtga, ttagctga, ctgtatga, cggattca, ggcattca, tgtggtca, tgacgtca, tgagctca, tgtcctca, cggtatca, ggctatca, cggttaca, ggcttaca, cgcataca, tgaggaca, tgtcgaca, tgtgcaca, or tgaccaca
  source 1..57: mol_type = other DNA
cgagacggatcaagcagaagacggcatacgagatnnnnnnnnnngtgactggagttc

SEQ ID NO: 93 - DNA, 120 bp, synthetic construct
  misc_feature 1..120: note = synthetic construct
  source 1..120: mol_type = other DNA
tcaacaagatgttttgccaactggccaagacctgccctgtgcagctgtgggttgattccacacccccgcccggcacccgcgtccgcgtcatgaccatctacaagcagtcacagcacatga

SEQ ID NO: 94 - DNA, 98 bp, synthetic construct
  misc_feature 1..98: note = synthetic construct
  source 1..98: mol_type = other DNA
tcacagcacatgacggaggttgtgaggcgctgccaccaccatgtgcgctgctcagatagcgatggtgagcagctggggctggagagacgacagggctg

SEQ ID NO: 95 - DNA, 119 bp, synthetic construct
  misc_feature 1..119: note = synthetic construct
  source 1..119: mol_type = other DNA
acagggctggttgcccagggtccccaggcctctgattcctcactgattgctcttaggtctggcccctcctcagcatcatatccgagtcgaaggaaatttgcgtgtggagtatttggatg

SEQ ID NO: 96 - DNA, 123 bp, synthetic construct
  misc_feature 1..123: note = synthetic construct
  source 1..123: mol_type = other DNA
gagtatttggatgacagaaacacttttcgacacagtgtggtgatgccctatgagccgcctgaggtctggtttgcaactggggtctctgggaggaggggttaagggtggttgtcagtggccctc

SEQ ID NO: 97 - DNA, 133 bp, synthetic construct
  misc_feature 1..133: note = synthetic construct
  source 1..133: mol_type = other DNA
ctggcctcatcttgggcctgtgttatctcctaggttggctctgactgtaccaccatccactacaacgacatgtgtaactgttcctgcatgggcggcatgaaccggaggcccatcctcaccatcatcacactgg

SEQ ID NO: 98 - DNA, 114 bp, synthetic construct
  misc_feature 1..114: note = synthetic construct
  source 1..114: mol_type = other DNA
tactgggacggaacagctttgaggtgcgtgtttgtgcatgtcctgggacagaccggcgcacagaggaagagaatctccgcaagaaaggggagcctcaccacgagctgcccccag

SEQ ID NO: 99 - DNA, 108 bp, synthetic construct
  misc_feature 1..108: note = synthetic construct
  source 1..108: mol_type = other DNA
caaagcaatttctacacgagatcctctctctgaagtcagtgagcaggagaaagattttctatggagtcacaggtaagtgctaaaatggagattctctgtttctttttc

SEQ ID NO: 100 - DNA, 112 bp, synthetic construct
  misc_feature 1..112: note = synthetic construct
  source 1..112: mol_type = other DNA
gaggctttggagtatttcatgaaacaaatgaatcatacacatcatggtggctggacaacaaaaatggattggatcttccacacaattaaacagcatgcattgaactgaaaag

SEQ ID NO: 101 - DNA, 109 bp, synthetic construct
  misc_feature 1..109: note = synthetic construct
  source 1..109: mol_type = other DNA
cctcacagtaaaaataggtgattttggtctagcgacagtgaaagctcgatggagtgggtcccatcagtttgaacagttgtctggatccattttgtggatggtaagaatt

SEQ ID NO: 102 - DNA, 96 bp, synthetic construct
  misc_feature 1..96: note = synthetic construct
  source 1..96: mol_type = other DNA
aagggcaactactggacgctggacccgacctgcgcagacatgttcgagaagggcaactaccggcgccgccgccgcatgaagaggcccttccggccg

SEQ ID NO: 103 - DNA, 103 bp, synthetic construct
  misc_feature 1..103: note = synthetic construct
  source 1..103: mol_type = other DNA
acctcaattttgtttcaggacctgcttcactgccgtatcctgacttctggaatctttgagaccaagttccaggtggacaaagtcaacttccagtaagccaact

SEQ ID NO: 104 - DNA, 123 bp, synthetic construct
  misc_feature 1..123: note = synthetic construct
  source 1..123: mol_type = other DNA
cactggcagcaacagtcttacctggactctggaatccattctgatgccactaccacagatccttctctgagtggtaaaggcaatcctgaggaagaggatgtggatacctcccaagtcctgtat

SEQ ID NO: 105 - DNA, 108 bp, synthetic construct
  misc_feature 1..108: note = synthetic construct
  source 1..108: mol_type = other DNA
ctgcctgctgcctcaggatccccgtccccgactcccaggtacttccggaacctgtgctcagatgacacccccacggtgcggcggaccgcagcctccaagctgggggag

SEQ ID NO: 106 - DNA, 100 bp, synthetic construct
  misc_feature 1..100: note = synthetic construct
  source 1..100: mol_type = other DNA
ctgcgccaggccgctgaagacaagacctggcgcatccgctacatggtggctgacaagttcacagaggtagatgagcgaccgttgacattgtcccactggt

SEQ ID NO: 107 - DNA, 116 bp, synthetic construct
  misc_feature 1..116: note = synthetic construct
  source 1..116: mol_type = other DNA
tgcagcaattcactgtaaagctggaaagggacgaacaggtgtaatgacatgtgcatatttattacatcggggcaaatttttaaaggcacaagaggccctagatttctatggggaag

SEQ ID NO: 108 - DNA, 111 bp, synthetic construct
  misc_feature 1..111: note = synthetic construct
  source 1..111: mol_type = other DNA
aggtgaagatatattcctccaattcaggaccctcacgacgggtagacaagttcatgtactttgagttccctcagccgttacctgtgtgtggtgatatcaaagtagagttct

SEQ ID NO: 109 - DNA, 118 bp, synthetic construct
  misc_feature 1..118: note = synthetic construct
  source 1..118: mol_type = other DNA
gagacttggcagccagaaatatcctccttactcatggtcggatcacaaagatttgtgattttggtctagccatagacatcacgaatgattctaattatgtggttaaaggaaacgtgag

SEQ ID NO: 110 - DNA, 129 bp, synthetic construct
  misc_feature 1..129: note = synthetic construct
  source 1..129: mol_type = other DNA
tatttttccctttctccccacagaaacctatgtatgaagtacagtggaaggatgttgaggagataaatggaaacaattatgtttacatagacccaacacaacttccttatgatcacaaatgggagtttc

SEQ ID NO: 111 - DNA, 108 bp, synthetic construct
  misc_feature 1..108: note = synthetic construct
  source 1..108: mol_type = other DNA
gttttcctgtagcaaaaccagaaatcctgacttacgacaggctagtgaatggcatgctccaatgtgtggcagcaggattcccagagcccacaatagattggtattttt

SEQ ID NO: 112 - DNA, 114 bp, synthetic construct
  misc_feature 1..114: note = synthetic construct
  source 1..114: mol_type = other DNA
agaaggtgagaaagttaaaattcccgtcgctatgaaggaattaagagaagcaacatctccgtaagccaacaaggaaatcctcgatgtgagtttctgctttgctgtgtgggggtc

SEQ ID NO: 113 - DNA, 102 bp, synthetic construct
  misc_feature 1..102: note = synthetic construct
  source 1..102: mol_type = other DNA
ccgcagcatgtcaagatcacagattttgggctggacaaacagctgggtgcggaagagaaagaataccatgcagaaggaggcaaagtaaggaggtggctttag

SEQ ID NO: 114 - DNA, 84 bp, synthetic construct
  misc_feature 1..84: note = synthetic construct
  source 1..84: mol_type = other DNA
gcctcacctccaccgtgcagctcatgacgtagctcatgcccttcggctgcctcctggactatgtccgggaacacaaagacaata

SEQ ID NO: 115 - DNA, 87 bp, synthetic construct
  misc_feature 1..87: note = synthetic construct
  source 1..87: mol_type = other DNA
tctcaccacccgcacgtctgtagaggactacatcaagacctggcggccacgctacttcctcctcaagaatgatggcaccttcattgg

SEQ ID NO: 116 - DNA, 116 bp, synthetic construct
  misc_feature 1..116: note = synthetic construct
  source 1..116: mol_type = other DNA
tgtacttccatacttgattcatgatattttactcctagatacgaatgaatcatggagaaatctgctttctacacatgttcagggatttttcaccagctgtcttcgacacttctcgc

SEQ ID NO: 117 - DNA, 97 bp, synthetic construct
  misc_feature 1..97: note = synthetic construct
  source 1..97: mol_type = other DNA
caccacctcctcaaacagctcaaaccatgcgataagtacctaaaaataaagcacctactgctgaaaagagagagagtggacctaagcaagctgcagt

SEQ ID NO: 118 - DNA, 104 bp, synthetic construct
  misc_feature 1..104: note = synthetic construct
  source 1..104: mol_type = other DNA
gctctgggagatcttcacgctgggggactccccgtatcccggcatccctgtggaggagctcttcaagctgctgaaggagggccaccgcatggacaagcccgcca

SEQ ID NO: 119 - DNA, 110 bp, synthetic construct
  misc_feature 1..110: note = synthetic construct
  source 1..110: mol_type = other DNA
tggcccctgagcgtcatctgcccccactgagcgctccacgcaccggcccatcctgcaggcggggctgccggccaaccagacggcggtgctgggcagcgacgtggagttcc

SEQ ID NO: 120 - DNA, 100 bp, synthetic construct
  misc_feature 1..100: note = synthetic construct
  source 1..100: mol_type = other DNA
aggagctggtggaggctgacgaggcgggcagtatgtatacaggcatcctcagctacggggtgggcttcttcctgttcatcctggtggtggcggctgtgac

SEQ ID NO: 121 - DNA, 115 bp, synthetic construct
  misc_feature 1..115: note = synthetic construct
  source 1..115: mol_type = other DNA
gcattccctatcaaatatgtcaacgacttcatcaacaagatagtcaacaaaaacaatgtgagatgtctccagcatttttacggacccaatcatgagcactgctttaatagggtaa

SEQ ID NO: 122 - DNA, 105 bp, synthetic construct
  misc_feature 1..105: note = synthetic construct
  source 1..105: mol_type = other DNA
gctgattttggtcttgccagagacatgtatcataaacaatactatagtgtacacaacaaaacaggtgcaaagctgccagtgaagtggatggctttggaaagtctg

SEQ ID NO: 123 - DNA, 130 bp, synthetic construct
  misc_feature 1..130: note = synthetic construct
  source 1..130: mol_type = other DNA
ccgcatcgactccaccgaggtcatctaccagccgagccgcatgcgggccaagctcatcggcaagtacctgatgggggacctgctgggggaaggctcttacggcaaggtgaaggaggtgctggactcggag

SEQ ID NO: 124 - DNA, 110 bp, synthetic construct
  misc_feature 1..110: note = synthetic construct
  source 1..110: mol_type = other DNA
ccgtacttggaggacctgcacggcgcggatgaggacgaggaccacttcgacatcgaggatgacatcatctacactcaggacttcacggtgcccggtgagtctggcggggg

SEQ ID NO: 125 - DNA, 107 bp, synthetic construct
  misc_feature 1..107: note = synthetic construct
  source 1..107: mol_type = other DNA
tatagtcacattttcattatttttattataaggcctgctgaaaatgactgaatataaacttgtggtagttgcagatggtggcgtaggcaagagtgccttgacgatac

SEQ ID NO: 126 - DNA, 109 bp, synthetic construct
  misc_feature 1..109: note = synthetic construct
  source 1..109: mol_type = other DNA
cttggatattctcgacacagcaggtcaagacgagtactgtgcaatgagggaccagtacatgaggactggggagggctttctttgtgtatttgccataaataatactaaa

SEQ ID NO: 127 - DNA, 125 bp, synthetic construct
  misc_feature 1..125: note = synthetic construct
  source 1..125: mol_type = other DNA
tgtagatgtggctcgccaattaaccctgattactggtttccaacaggttcttgctggtgtgaaatgactgagtacaaactggtcgtggatggagcaggtggtgttgggaaaagcgcactgacaat

SEQ ID NO: 128 - DNA, 113 bp, synthetic construct
  misc_feature 1..113: note = synthetic construct
  source 1..113: mol_type = other DNA
gttggacatactggatacagctggacaagaagagcacagtgacatgagagaccaatacatgaggacaggcgaaggcttcctctgtgtatttgccatcaataatagcaagtcat

SEQ ID NO: 129 - DNA, 118 bp, synthetic construct
  misc_feature 1..118: note = synthetic construct
  source 1..118: mol_type = other DNA
ggtggggcaggagaccctgtaggaggaccccgggccgcaggcccctgaggagcgatgacggaatataagctggtggtcgtggacgccggcggtgtgggcaagagtgcgctgaccatcc

SEQ ID NO: 130 - DNA, 118 bp, synthetic construct
  misc_feature 1..118: note = synthetic construct
  source 1..118: mol_type = other DNA
tggacatcctggataccgccggccaggagtactacagcgccatgcgggaccagtacatgcgcaccggggagggcttcctgtgtgtgtttgccatcaacaacaccaagtcttttgagga

SEQ ID NO: 131 - DNA, 122 bp, synthetic construct
  misc_feature 1..122: note = synthetic construct
  source 1..122: mol_type = other DNA
tgttcctgtttataatattgacaaaacaccttagcggatgacatttaagaattctaaaagtcctaatatatgtaatatatattcagttgcctgaagagaaacataaagaatcctttcttaat

SEQ ID NO: 132 - DNA, 119 bp, synthetic construct
  misc_feature 1..119: note = synthetic construct
  source 1..119: mol_type = other DNA
atgtcaggacaaagtccggattgaatataactctgctttatattataggcctatgaagaatacaccagcaagctagatgcactccaacaaagagaacaacagttattggaatctctggg

SEQ ID NO: 133 - DNA, 22 bp, synthetic construct
  misc_feature 1..22: note = synthetic construct
  source 1..22: mol_type = other DNA
caagatgttttgccaactggcc

SEQ ID NO: 134 - DNA, 22 bp, synthetic construct
  misc_feature 1..22: note = synthetic construct
  source 1..22: mol_type = other DNA
cctgtcgtctctccagccccag

SEQ ID NO: 135 - DNA, 20 bp, synthetic construct
  misc_feature 1..20: note = synthetic construct
  source 1..20: mol_type = other DNA
ggctggttgcccagggtccc

SEQ ID NO: 136 - DNA, 23 bp, synthetic construct
  misc_feature 1..23: note = synthetic construct
  source 1..23: mol_type = other DNA
gccactgacaaccacccttaacc

SEQ ID NO: 137 - DNA, 24 bp, synthetic construct
  misc_feature 1..24: note = synthetic construct
  source 1..24: mol_type = other DNA
cctcatcttgggcctgtgttatct

SEQ ID NO: 138 - DNA, 19 bp, synthetic construct
  misc_feature 1..19: note = synthetic construct
  source 1..19: mol_type = other DNA
gggcagctcgtggtgaggc

SEQ ID NO: 139 - DNA, 28 bp, synthetic construct
  misc_feature 1..28: note = synthetic construct
  source 1..28: mol_type = other DNA
aagaaacagagaatctccattttagcac

SEQ ID NO: 140 - DNA, 28 bp, synthetic construct
  misc_feature 1..28: note = synthetic construct
  source 1..28: mol_type = other DNA
tcagttcaatgcatgctgtttaattgtg

SEQ ID NO: 141 - DNA, 27 bp, synthetic construct
  misc_feature 1..27: note = synthetic construct
  source 1..27: mol_type = other DNA
cttaccatccacaaaatggatccagac

SEQ ID NO: 142 - DNA, 22 bp, synthetic construct
  misc_feature 1..22: note = synthetic construct
  source 1..22: mol_type = other DNA
cggaagggcctcttcatgcggc

SEQ ID NO: 143 - DNA, 27 bp, synthetic construct
  misc_feature 1..27: note = synthetic construct
  source 1..27: mol_type = other DNA
ggcttactggaagttgactttgtccac

SEQ ID NO: 144 - DNA, 24 bp, synthetic construct
  misc_feature 1..24: note = synthetic construct
  source 1..24: mol_type = other DNA
aggacttgggaggtatccacatcc

SEQ ID NO: 145 - DNA, 23 bp, synthetic construct
  misc_feature 1..23: note = synthetic construct
  source 1..23: mol_type = other DNA
ctgctgcctcaggatccccgtcc

SEQ ID NO: 146 - DNA, 23 bp, synthetic construct
  misc_feature 1..23: note = synthetic construct
  source 1..23: mol_type = other DNA
gtgggacaatgtcaacggtcgct

SEQ ID NO: 147 - DNA, 22 bp, synthetic construct
  misc_feature 1..22: note = synthetic construct
  source 1..22: mol_type = other DNA
cccatagaaatctagggcctct

SEQ ID NO: 148 - DNA, 27 bp, synthetic construct
  misc_feature 1..27: note = synthetic construct
  source 1..27: mol_type = other DNA
ctctactttgatatcaccacacacagg

SEQ ID NO: 149 - DNA, 29 bp, synthetic construct
  misc_feature 1..29: note = synthetic construct
  source 1..29: mol_type = other DNA
cttggcagccagaaatatcctccttactc

SEQ ID NO: 150 - DNA, 27 bp, synthetic construct
  misc_feature 1..27: note = synthetic construct
  source 1..27: mol_type = other DNA
ctcccatttgtgatcataaggaagttg

SEQ ID NO: 151 - DNA, 24 bp, synthetic construct
  misc_feature 1..24: note = synthetic construct
  source 1..24: mol_type = other DNA
ataccaatctattgtgggctctgg

SEQ ID NO: 152 - DNA, 21 bp, synthetic construct
  misc_feature 1..21: note = synthetic construct
  source 1..21: mol_type = other DNA
cccacacagcaaagcagaaac

SEQ ID NO: 153 - DNA, 23 bp, synthetic construct
  misc_feature 1..23: note = synthetic construct
  source 1..23: mol_type = other DNA
agccacctccttactttgcctcc

SEQ ID NO: 154 - DNA, 25 bp, synthetic construct
  misc_feature 1..25: note = synthetic construct
  source 1..25: mol_type = other DNA
gtctttgtgttcccggacatagtcc

SEQ ID NO: 155 - DNA, 25 bp, synthetic construct
  misc_feature 1..25: note = synthetic construct
  source 1..25: mol_type = other DNA
tgaaggtgccatcattcttgaggag

SEQ ID NO: 156 - DNA, 24 bp, synthetic construct
  misc_feature 1..24: note = synthetic construct
  source 1..24: mol_type = other DNA
gaagtgtcgaagacagctggtgaa

SEQ ID NO: 157 - DNA, 24 bp, synthetic construct
  misc_feature 1..24: note = synthetic construct
  source 1..24: mol_type = other DNA
cagcttgcttaggtccactctctc

SEQ ID NO: 158 - DNA, 21 bp, synthetic construct
  misc_feature 1..21: note = synthetic construct
  source 1..21: mol_type = other DNA
gggcttgtccatgcggtggcc

SEQ ID NO: 159 - DNA, 22 bp, synthetic construct
  misc_feature 1..22: note = synthetic construct
  source 1..22: mol_type = other DNA
ctccacgtcgctgcccagcacc

SEQ ID NO: 160 - DNA, 23 bp, synthetic construct
  misc_feature 1..23: note = synthetic construct
  source 1..23: mol_type = other DNA
cagccgccaccaccaggatgaac

SEQ ID NO: 161 - DNA, 26 bp, synthetic construct
  misc_feature 1..26: note = synthetic construct
  source 1..26: mol_type = other DNA
cctattaaagcagtgctcatgattgg

SEQ ID NO: 162 - DNA, 23 bp, synthetic construct
  misc_feature 1..23: note = synthetic construct
  source 1..23: mol_type = other DNA
ctttccaaagccatccacttcac

SEQ ID NO: 163 - DNA, 24 bp, synthetic construct
  misc_feature 1..24: note = synthetic construct
  source 1..24: mol_type = other DNA
gagtccagcacctccttcaccttg

SEQ ID NO: 164 - DNA, 20 bp, synthetic construct
  misc_feature 1..20: note = synthetic construct
  source 1..20: mol_type = other DNA
cgccagactcaccgggcacc

SEQ ID NO: 165 - DNA, 34 bp, synthetic construct
  misc_feature 1..34: note = synthetic construct
  source 1..34: mol_type = other DNA
gtcacattttcattatttttattataaggcctgc

SEQ ID NO: 166 - DNA, 31 bp, synthetic construct
  misc_feature 1..31: note = synthetic construct
  source 1..31: mol_type = other DNA
gtattatttatggcaaatacacaaagaaagc

SEQ ID NO: 167 - DNA, 25 bp, synthetic construct
  misc_feature 1..25: note = synthetic construct
  source 1..25: mol_type = other DNA
gatgtggctcgccaattaaccctga

SEQ ID NO: 168 - DNA, 28 bp, synthetic construct
  misc_feature 1..28: note = synthetic construct
  source 1..28: mol_type = other DNA
cttgctattattgatggcaaatacacag

SEQ ID NO: 169 - DNA, 21 bp, synthetic construct
  misc_feature 1..21: note = synthetic construct
  source 1..21: mol_type = other DNA
gggcaggagaccctgtaggag

SEQ ID NO: 170 - DNA, 26 bp, synthetic construct
  misc_feature 1..26: note = synthetic construct
  source 1..26: mol_type = other DNA
caaaagacttggtgttgttgatggca

SEQ ID NO: 171 - DNA, 29 bp, synthetic construct
  misc_feature 1..29: note = synthetic construct
  source 1..29: mol_type = other DNA
agaaaggattctttatgtttctcttcagg

SEQ ID NO: 172 - DNA, 29 bp, synthetic construct
  misc_feature 1..29: note = synthetic construct
  source 1..29: mol_type = other DNA
gagattccaataactgttgttctctttgt

SEQ ID NO: 173 - DNA, 23 bp, synthetic construct
  misc_feature 1..23: note = primer
  source 1..23: mol_type = other DNA
cgagacggatcaagcagaagacg

SEQ ID NO: 174 - DNA, 20 bp, synthetic construct
  misc_feature 1..20: note = primer
  source 1..20: mol_type = other DNA
gccaactggccaagacctgc

SEQ ID NO: 175 - DNA, 20 bp, synthetic construct
  misc_feature 1..20: note = primer
  source 1..20: mol_type = other DNA
ctccagccccagctgctcac

SEQ ID NO: 176 - DNA, 22 bp, synthetic construct
  misc_feature 1..22: note = primer
  source 1..22: mol_type = other DNA
gtccccaggcctctgattcctc

SEQ ID NO: 177 - DNA, 21 bp, synthetic construct
  misc_feature 1..21: note = primer
  source 1..21: mol_type = other DNA
cctcccagagaccccagttgc

SEQ ID NO: 178 - DNA, 25 bp, synthetic construct
  misc_feature 1..25: note = primer
  source 1..25: mol_type = other DNA
tgggcctgtgttatctcctaggttg

SEQ ID NO: 179 - DNA, 20 bp, synthetic construct
  misc_feature 1..20: note = primer
  source 1..20: mol_type = other DNA
gcagctcgtggtgaggctcc

SEQ ID NO: 180 - DNA, 32 bp, synthetic construct
  misc_feature 1..32: note = primer
  source 1..32: mol_type = other DNA
agaaacagagaatctccattttagcacttacc

SEQ ID NO: 181 - DNA, 30 bp, synthetic construct
  misc_feature 1..30: note = primer
  source 1..30: mol_type = other DNA
ttcaatgcatgctgtttaattgtgtggaag

SEQ ID NO: 182 - DNA, 28 bp, synthetic construct
  misc_feature 1..28: note = primer
  source 1..28: mol_type = other DNA
tccacaaaatggatccagacaactgttc

SEQ ID NO: 183 - DNA, 20 bp, synthetic construct
  misc_feature 1..20: note = primer
  source 1..20: mol_type = other DNA
ggcgccggtagttgcccttc

SEQ ID NO: 184 - DNA, 26 bp, synthetic construct
  misc_feature 1..26: note = primer
  source 1..26: mol_type = other DNA
ggaagttgactttgtccacctggaac

SEQ ID NO: 185 - DNA, 26 bp, synthetic construct
  misc_feature 1..26: note = primer
  source 1..26: mol_type = other DNA
ggaggtatccacatcctcttcctcag

SEQ ID NO: 186 - DNA, 23 bp, synthetic construct
  misc_feature 1..23: note = primer
  source 1..23: mol_type = other DNA
cgactcccaggtacttccggaac

SEQ ID NO: 187 - DNA, 24 bp, synthetic construct
  misc_feature 1..24: note = primer
  source 1..24: mol_type = other DNA
tgtcaacggtcgctcatctacctc

SEQ ID NO: 188 - DNA, 26 bp, synthetic construct
  misc_feature 1..26: note = primer
  source 1..26: mol_type = other DNA
ccatagaaatctagggcctcttgtgc

SEQ ID NO: 189 - DNA, 22 bp, synthetic construct
  misc_feature 1..22: note = primer
  source 1..22: mol_type = other DNA
caccacacacaggtaacggctg

SEQ ID NO: 190 - DNA, 30 bp, synthetic construct
  misc_feature 1..30: note = primer
  source 1..30: mol_type = other DNA
gaaatatcctccttactcatggtcggatca

SEQ ID NO: 191 - DNA, 30 bp, synthetic construct
  misc_feature 1..30: note = primer
  source 1..30: mol_type = other DNA
cccatttgtgatcataaggaagttgtgttg

SEQ ID NO: 192 - DNA, 22 bp, synthetic construct
  misc_feature 1..22: note = primer
  source 1..22: mol_type = other DNA
gtgggctctgggaatcctgctg

SEQ ID NO: 193 - DNA, 24 bp, synthetic construct
  misc_feature 1..24: note = primer
  source 1..24: mol_type = other DNA
ccacacagcaaagcagaaactcac

SEQ ID NO: 194 - DNA, 25 bp, synthetic construct
  misc_feature 1..25: note = primer
  source 1..25: mol_type = other DNA
acctccttactttgcctccttctgc

SEQ ID NO: 195 - DNA, 24 bp, synthetic construct
  misc_feature 1..24: note = primer
  source 1..24: mol_type = other DNA
gtgttcccggacatagtccaggag

SEQ ID NO: 196 - DNA, 26 bp, synthetic construct
  misc_feature 1..26: note = primer
  source 1..26: mol_type = other DNA
gccatcattcttgaggaggaagtagc

SEQ ID NO: 197 - DNA, 26 bp, synthetic construct
  misc_feature 1..26: note = primer
  source 1..26: mol_type = other DNA
agacagctggtgaaaaatccctgaac

SEQ ID NO: 198 - DNA, 28 bp, synthetic construct
  misc_feature 1..28: note = primer
  source 1..28: mol_type = other DNA
tgcttaggtccactctctctcttttcag

SEQ ID NO: 199 - DNA, 21 bp, synthetic construct
  misc_feature 1..21: note = primer
  source 1..21: mol_type = other DNA
gcggtggccctccttcagcag

SEQ ID NO: 200 - DNA, 20 bp, synthetic construct
  misc_feature 1..20: note = primer
  source 1..20: mol_type = other DNA
ccagcaccgccgtctggttg

SEQ ID NO: 201 - DNA, 24 bp, synthetic construct
  misc_feature 1..24: note = primer
  source 1..24: mol_type = other DNA
ccaccaggatgaacaggaagaagc

SEQ ID NO: 202 - DNA, 22 bp, synthetic construct
  misc_feature 1..22: note = primer
  source 1..22: mol_type = other DNA
cagtgctcatgattgggtccgt

SEQ ID NO: 203 - DNA, 21 bp, synthetic construct
  misc_feature 1..21: note = primer
  source 1..21: mol_type = other DNA
gccatccacttcactggcagc

SEQ ID NO: 204 - DNA, 24 bp, synthetic construct
  misc_feature 1..24: note = primer
  source 1..24: mol_type = other DNA
cttcaccttgccgtaagagccttc

SEQ ID NO: 205 - DNA, 21 bp, synthetic construct
  misc_feature 1..21: note = primer
  source 1..21: mol_type = other DNA
ctcaccgggcaccgtgaagtc

SEQ ID NO: 206 - DNA, 37 bp, synthetic construct
  misc_feature 1..37: note = primer
  source 1..37: mol_type = other DNA
cattatttttattataaggcctgctgaaaatgactga

SEQ ID NO: 207 - DNA, 26 bp, synthetic construct
  misc_feature 1..26: note = primer
  source 1..26: mol_type = other DNA
tggcaaatacacaaagaaagccctcc

SEQ ID NO: 208 - DNA, 30 bp, synthetic construct
  misc_feature 1..30: note = primer
  source 1..30: mol_type = other DNA
caattaaccctgattactggtttccaacag

SEQ ID NO: 209 - DNA, 25 bp, synthetic construct
  misc_feature 1..25: note = primer
  source 1..25: mol_type = other DNA
ggcaaatacacagaggaagccttcg

SEQ ID NO: 210 - DNA, 22 bp, synthetic construct
  misc_feature 1..22: note = primer
  source 1..22: mol_type = other DNA
caggagaccctgtaggaggacc

SEQ ID NO: 211 - DNA, 24 bp, synthetic construct
  misc_feature 1..24: note = primer
  source 1..24: mol_type = other DNA
tgatggcaaacacacacaggaagc

SEQ ID NO: 212 - DNA, 31 bp, synthetic construct
  misc_feature 1..31: note = primer
  source 1..31: mol_type = other DNA
aggattctttatgtttctcttcaggcaactg

SEQ ID NO: 213 - DNA, 28 bp, synthetic construct
  misc_feature 1..28: note = primer
  source 1..28: mol_type = other DNA
actgttgttctctttgttggagtgcatc

SEQ ID NO: 214 - DNA, 23 bp, synthetic construct
  misc_feature 1..23: note = synthetic construct
  source 1..23: mol_type = other DNA
cgagacggatcaagcagaagacg

SEQ ID NO: 215 - DNA, 61 bp, synthetic construct
  misc_feature 1..61: note = synthetic construct
  misc_difference 35..42: note = this stretch nnnnnnnn represents any one of the 96 barcode sequences defined in Table 6
  misc_difference 43..48: note = n is a, c, g, or t
  source 1..61: mol_type = other DNA
cgagacggatcaagcagaagacggcatacgagatnnnnnnnnnnnnnngtgactggagttc

SEQ ID NO: 216 - DNA, 78 bp, synthetic construct
  misc_feature 1..78: note = synthetic construct
  source 1..78: mol_type = other DNA
atcggaagagcacacgtctgaactccagtcacaaaaaaaaaaaaatctcgtatgccgtcttctgcttgatccgtctcg

SEQ ID NO: 217 - DNA, 23 bp, synthetic construct
  misc_feature 1..23: note = primer
  source 1..23: mol_type = other DNA
caagcagaagacggcatacgaga

SEQ ID NO: 218 - DNA, 87 bp, synthetic construct
  misc_feature 1..87: note = synthetic construct
  misc_difference 59..62: note = n is a, c, g, or t
  source 1..87: mol_type = other DNA
aatgatacggcgaccaccgagatctacactctttccctacacgacgctcttccgatctnnnnacttgcagctgtgggttgattccac

SEQ ID NO: 219 - DNA, 88 bp, synthetic construct
  misc_feature 1..88: note = synthetic construct
  misc_difference 59..62: note = n is a, c, g, or t
  source 1..88: mol_type = other DNA
aatgatacggcgaccaccgagatctacactctttccctacacgacgctcttccgatctnnnnactccagctgctcaccatcgctatct

SEQ ID NO: 220 - DNA, 91 bp, synthetic construct
  misc_feature 1..91: note = synthetic construct
  misc_difference 59..62: note = n is a, c, g, or t
  source 1..91: mol_type = other DNA
aatgatacggcgaccaccgagatctacactctttccctacacgacgctcttccgatctnnnnacttcctcactgattgctcttaggtctgg

SEQ ID NO: 221 - DNA, 87 bp, synthetic construct
  misc_feature 1..87: note = synthetic construct
  misc_difference 59..62: note = n is a, c, g, or t
  source 1..87: mol_type = other DNA
aatgatacggcgaccaccgagatctacactctttccctacacgacgctcttccgatctnnnnactcaaaccagacctcaggcggctc

SEQ ID NO: 222 - DNA, 88 bp, synthetic construct
  misc_feature 1..88: note = synthetic construct
  misc_difference 59..62: note = n is a, c, g, or t
  source 1..88: mol_type = other DNA
aatgatacggcgaccaccgagatctacactctttccctacacgacgctcttccgatctnnnnactgctctgactgtaccaccatccac

SEQ ID NO: 223 - DNA, 89 bp, synthetic construct
  misc_feature 1..89: note = synthetic construct
  misc_difference 59..62: note = n is a, c, g, or t
  source 1..89: mol_type = other DNA
aatgatacggcgaccaccgagatctacactctttccctacacgacgctcttccgatctnnnnacttcccctttcttgcggagattctct

SEQ ID NO: 224 - DNA, 96 bp, synthetic construct
  misc_feature 1..96: note = synthetic construct
  misc_difference 59..62: note = n is a, c, g, or t
  source 1..96: mol_type = other DNA
aatgatacggcgaccaccgagatctacactctttccctacacgacgctcttccgatctnnnnactcacttacctgtgactccatagaaaatctttc

SEQ ID NO: 225 - DNA, 94 bp, synthetic construct
  misc_feature 1..94: note = synthetic construct
  misc_difference 59..62: note = n is a, c, g, or t
  source 1..94: mol_type = other DNA
aatgatacggcgaccaccgagatctacactctttccctacacgacgctcttccgatctnnnnactgaagatccaatccatttttgttgtccagc

SEQ ID NO: 226 - DNA, 89 bp, synthetic construct
  misc_feature 1..89: note = synthetic construct
  misc_difference 59..62: note = n is a, c, g, or t
  source 1..89: mol_type = other DNA
aatgatacggcgaccaccgagatctacactctttccctacacgacgctcttccgatctnnnnactcaaactgatgggacccactccatc

SEQ ID NO: 227 - DNA, 89 bp, synthetic construct
  misc_feature 1..89: note = synthetic construct
  misc_difference 59..62: note = n is a, c, g, or t
  source 1..89: mol_type = other DNA
aatgatacggcgaccaccgagatctacactctttccctacacgacgctcttccgatctnnnnactcggtagttgcccttctcgaacatg

SEQ ID NO: 228 - DNA, 93 bp, synthetic construct
  misc_feature 1..93: note = synthetic construct
  misc_difference 59..62: note = n is a, c, g, or t
  source 1..93: mol_type = other DNA
aatgatacggcgaccaccgagatctacactctttccctacacgacgctcttccgatctnnnnactacttggtctcaaagattccagaagtcag

SEQ ID NO: 229 - DNA, 90 bp, synthetic construct
  misc_feature 1..90: note = synthetic construct
  misc_difference 59..62: note = n is a, c, g, or t
  source 1..90: mol_type = other DNA
aatgatacggcgaccaccgagatctacactctttccctacacgacgctcttccgatctnnnnactcctcaggattgcctttaccactcag

SEQ ID NO: 230 - DNA, 89 bp, synthetic construct
  misc_feature 1..89: note = synthetic construct
  misc_difference 59..62: note = n is a, c, g, or t
  source 1..89: mol_type = other DNA
aatgatacggcgaccaccgagatctacactctttccctacacgacgctcttccgatctnnnnactccggaacctgtgctcagatgacac

SEQ ID NO: 231 - DNA, 91 bp, synthetic construct
  misc_feature 1..91: note = synthetic construct
  misc_difference 59..62: note = n is a, c, g, or t
  source 1..91: mol_type = other DNA
aatgatacggcgaccaccgagatctacactctttccctacacgacgctcttccgatctnnnnactctgtgaacttgtcagccaccatgtag

SEQ ID NO: 232 - DNA, 99 bp, synthetic construct
  misc_feature 1..99: note = synthetic construct
  misc_difference 59..62: note = n is a, c, g, or t
  source 1..99: mol_type = other DNA
aatgatacggcgaccaccgagatctacactctttccctacacgacgctcttccgatctnnnnactcctttaaaaatttgccccgatgtaataaatatgc

SEQ ID NO: 233 - DNA, 92 bp, synthetic construct
  misc_feature 1..92: note = synthetic construct
  misc_difference 59..62: note = n is a, c, g, or t
  source 1..92: mol_type = other DNA
aatgatacggcgaccaccgagatctacactctttccctacacgacgctcttccgatctnnnnactggctgagggaactcaaagtacatgaac

SEQ ID NO: 234 - DNA, 96 bp, synthetic construct
  misc_feature 1..96: note = synthetic construct
  misc_difference 59..62: note = n is a, c, g, or t
  source 1..96: mol_type = other DNA
aatgatacggcgaccaccgagatctacactctttccctacacgacgctcttccgatctnnnnactggatcacaaagatttgtgattttggtctagc

SEQ ID NO: 235 - DNA, 98 bp, synthetic construct
  misc_feature 1..98: note = synthetic construct
  misc_difference 59..62: note = n is a, c, g, or t
  source 1..98: mol_type = other DNA
aatgatacggcgaccaccgagatctacactctttccctacacgacgctcttccgatctnnnnactggtctatgtaaacataattgtttccatttatct

SEQ ID NO: 236 - DNA, 86 bp, synthetic construct
  misc_feature 1..86: note = synthetic construct
  misc_difference 59..62: note = n is a, c, g, or t
  source 1..86: mol_type = other DNA
aatgatacggcgaccaccgagatctacactctttccctacacgacgctcttccgatctnnnnactgccacacattggagcatgcca

SEQ ID NO: 237 - DNA, 93 bp, synthetic construct
  misc_feature 1..93: note = synthetic construct
  misc_difference 59..62: note = n is a, c, g, or t
  source 1..93: mol_type = other DNA
aatgatacggcgaccaccgagatctacactctttccctacacgacgctcttccgatctnnnnactgaaactcacatcgaggatttccttgttg

SEQ ID NO: 238 - DNA, 91 bp, synthetic construct
  misc_feature 1..91: note = synthetic construct
  misc_difference 59..62: note = n is a, c, g, or t
  source 1..91: mol_type = other DNA
aatgatacggcgaccaccgagatctacactctttccctacacgacgctcttccgatctnnnnacttgcatggtattctttctcttccgcac

SEQ ID NO: 239 - DNA, 86 bp, synthetic construct
  misc_feature 1..86: note = synthetic construct
  misc_difference 59..62: note = n is a, c, g, or t
  source 1..86: mol_type = other DNA
aatgatacggcgaccaccgagatctacactctttccctacacgacgctcttccgatctnnnnactgaggcagccgaagggcatgag

SEQ ID NO: 240 - DNA, 85 bp, synthetic construct
  misc_feature 1..85: note = synthetic construct
  misc_difference 59..62: note = n is a, c, g, or t
  source 1..85: mol_type = other DNA
aatgatacggcgaccaccgagatctacactctttccctacacgacgctcttccgatctnnnnactgtggccgccaggtcttgatg

SEQ ID NO: 241 - DNA, 96 bp, synthetic construct
  misc_feature 1..96: note = synthetic construct
  misc_difference 59..62: note = n is a, c, g, or t
  source 1..96: mol_type = other DNA
aatgatacggcgaccaccgagatctacactctttccctacacgacgctcttccgatctnnnnactcatgtgtagaaagcagatttctccatgattc

SEQ ID NO: 242 - DNA, 95 bp, synthetic construct
  misc_feature 1..95: note = synthetic construct
  misc_difference 59..62: note = n is a, c, g, or t
  source 1..95: mol_type = other DNA
aatgatacggcgaccaccgagatctacactctttccctacacgacgctcttccgatctnnnnactttcagcagtaggtgctttatttttaggtac

SEQ ID NO: 243 - DNA, 88 bp, synthetic construct
  misc_feature 1..88: note = synthetic construct
  misc_difference 59..62: note = n is a, c, g, or t
  source 1..88: mol_type = other DNA
aatgatacggcgaccaccgagatctacactctttccctacacgacgctcttccgatctnnnnactcagcttgaagagctcctccacag

SEQ ID NO: 244 - DNA, 84 bp, synthetic construct
  misc_feature 1..84: note = synthetic construct
  misc_difference 59..62: note = n is a, c, g, or t
  source 1..84: mol_type = other DNA
aatgatacggcgaccaccgagatctacactctttccctacacgacgctcttccgatctnnnnactcctgcaggatgggccggtg

SEQ ID NO: 245 - DNA, 86 bp, synthetic construct
  misc_feature 1..86: note = synthetic construct
  misc_difference 59..62: note = n is a, c, g, or t
  source 1..86: mol_type = other DNA
aatgatacggcgaccaccgagatctacactctttccctacacgacgctcttccgatctnnnnactccaccccgtagctgaggatgc

SEQ ID NO: 246 - DNA, 94 bp, synthetic construct
  misc_feature 1..94: note = synthetic construct
  misc_difference 59..62: note = n is a, c, g, or t
  source 1..94: mol_type = other DNA
aatgatacggcgaccaccgagatctacactctttccctacacgacgctcttccgatctnnnnactgctggagacatctcacattgtttttgttg

SEQ ID NO: 247 - DNA, 92 bp, synthetic construct
  misc_feature 1..92: note = synthetic construct
  misc_difference 59..62: note = n is a, c, g, or t
  source 1..92: mol_type = other DNA
aatgatacggcgaccaccgagatctacactctttccctacacgacgctcttccgatctnnnnactgctttgcacctgttttgttgtgtacac

SEQ ID NO: 248 - DNA, 88 bp, synthetic construct
  misc_feature 1..88: note = synthetic construct
  misc_difference 59..62: note = n is a, c, g, or t
  source 1..88: mol_type = other DNA
aatgatacggcgaccaccgagatctacactctttccctacacgacgctcttccgatctnnnnactcccatcaggtacttgccgatgag

SEQ ID NO: 249 - DNA, 93 bp, synthetic construct
  misc_feature 1..93: note = synthetic construct
  misc_difference 59..62: note = n is a, c, g, or t
  source 1..93: mol_type = other DNA
aatgatacggcgaccaccgagatctacactctttccctacacgacgctcttccgatctnnnnactctgagtgtagatgatgtcatcctcgatg

SEQ ID NO: 250 - DNA, 96 bp, synthetic construct
  misc_feature 1..96: note = synthetic construct
  misc_difference 59..62: note = n is a, c, g, or t
  source 1..96: mol_type = other DNA
aatgatacggcgaccaccgagatctacactctttccctacacgacgctcttccgatctnnnnactgctgaaaatgactgaatataaacttgtggta

SEQ ID NO: 251 - DNA, 91 bp, synthetic construct
  misc_feature 1..91: note = synthetic construct
  misc_difference 59..62: note = n is a, c, g, or t
  source 1..91: mol_type = other DNA
aatgatacggcgaccaccgagatctacactctttccctacacgacgctcttccgatctnnnnactcagtcctcatgtactggtccctcatt

SEQ ID NO: 252 - DNA, 92 bp, synthetic construct
  misc_feature 1..92: note = synthetic construct
  misc_difference 59..62: note = n is a, c, g, or t
  source 1..92: mol_type = other DNA
aatgatacggcgaccaccgagatctacactctttccctacacgacgctcttccgatctnnnnacttcttgctggtgtgaaatgactgagtac

SEQ ID NO: 253 - DNA, 89 bp, synthetic construct
  misc_feature 1..89: note = synthetic construct
  misc_difference 59..62: note = n is a, c, g, or t
  source 1..89: mol_type = other DNA
aatgatacggcgaccaccgagatctacactctttccctacacgacgctcttccgatctnnnnacttcgcctgtcctcatgtattggtct

SEQ ID NO: 254 - DNA, 91 bp, synthetic construct
  misc_feature 1..91: note = synthetic construct
  misc_difference 59..62: note = n is a, c, g, or t
  source 1..91: mol_type = other DNA
aatgatacggcgaccaccgagatctacactctttccctacacgacgctcttccgatctnnnnactcctgaggagcgatgacggaatataag

SEQ ID NO: 255 - DNA, 86 bp, synthetic construct
  misc_feature 1..86: note = synthetic construct
  misc_difference 59..62: note = n is a, c, g, or t
  source 1..86: mol_type = other DNA
aatgatacggcgaccaccgagatctacactctttccctacacgacgctcttccgatctnnnnactatgtactggtcccgcatggcg

SEQ ID NO: 256 - DNA, 96 bp, synthetic construct
  misc_feature 1..96: note = synthetic construct
  misc_difference 59..62: note = n is a, c, g, or t
  source 1..96: mol_type = other DNA
aatgatacggcgaccaccgagatctacactctttccctacacgacgctcttccgatctnnnnactaggacttttagaattcttaaatgtcatccgc

SEQ ID NO: 257 - DNA, 93 bp, synthetic construct
  misc_feature 1..93: note = synthetic construct
  misc_difference 59..62: note = n is a, c, g, or t
  source 1..93: mol_type = other DNA
aatgatacggcgaccaccgagatctacactctttccctacacgacgctcttccgatctnnnnacttctagcttgctggtgtattcttcatagg

SEQ ID NO: 258 - DNA, 79 bp, Homo sapiens
  source 1..79: mol_type = other DNA
tgcagctgtgggttgattccacacccccgcccggcacccgcgtccgcgccatggccatctacaagcagtcacagcacag

SEQ ID NO: 259 - DNA, 73 bp, Homo sapiens
  source 1..73: mol_type = other DNA
ccagctgctcaccatcgctatctgagcagcgctcatggtgggggcagcgcctcacaacctccgtcatgtgcta

SEQ ID NO: 260 - DNA, 80 bp, Homo sapiens
  source 1..80: mol_type = other DNA
tcctcactgattgctcttaggtctggcccctcctcagcatcttatccgagtggaaggaaatttgcgtgtggagtatttgt

SEQ ID NO: 261 - DNA, 71 bp, Homo sapiens
  source 1..71: mol_type = other DNA
caaaccagacctcaggcggctcatagggcaccaccacactatgtcgaaaagtgtttctgtcatccaaatat

SEQ ID NO: 262 - DNA, 93 bp, Homo sapiens
  source 1..93: mol_type = other DNA
gctctgactgtaccaccatccactacaactacatgtgtaacagttcctgcatgggcggcatgaaccggaggcccatcctcaccatcatcacag

SEQ ID NO: 263 - DNA, 89 bp, Homo sapiens
  source 1..89: mol_type = other DNA
tcccctttcttgcggagattctcttcctctgtgcgccggtctctcccaggacaggcacaaacacgcacctcaaagctgttccgtcccag

SEQ ID NO: 264 - DNA, 76 bp, Homo sapiens
  source 1..76: mol_type = other DNA
cacttacctgtgactccatagaaaatctttctcctgctcagtgatttcagagagaggatctcgtgtagaaattgca

SEQ ID NO: 265 - DNA, 75 bp, Homo sapiens
  source 1..75: mol_type = other DNA
gaagatccaatccatttttgttgtccagccaccatgatgtgcatcattcatttgtttcatgaaatactccaaaga

SEQ ID NO: 266 - DNA, 68 bp, Homo sapiens
  source 1..68: mol_type = other DNA
caaactgatgggacccactccatcgagatttcactgtagctagaccaaaatcacctatttttactgtt

SEQ ID NO: 267 - DNA, 59 bp, Homo sapiens
  source 1..59: mol_type = other DNA
cggtagttgcccttctcgaacatgtcttcgcaggccgggtccagcgtccagtagttgca

SEQ ID NO: 268 - DNA, 65 bp, Homo sapiens
  source 1..65: mol_type = other DNA
acttggtctcaaagattccagaagtcaggacacggcagcgaagcaggtcctgaaacaaaattgag

SEQ ID NO: 269 - DNA, 89 bp, Homo sapiens
  source 1..89: mol_type = other DNA
cctcaggattgcctttaccactcagagaaggagctgtggtagtggcaccagaatggattccagagtccaggtaagactgttgctgccag

SEQ ID NO: 270 - DNA, 61 bp, Homo sapiens
  source 1..61: mol_type = other DNA
ccggaacctgtgctcagatgacacccccatggtgcggcgggccgcagcctccaagctgggt

SEQ ID NO: 271 - DNA, 61 bp, Homo sapiens
  source 1..61: mol_type = other DNA
ctgtgaacttgtcagccaccatgtagcggacgcgccaggacttgtcttcagcggcctggct

SEQ ID NO: 272 - DNA, 83 bp, Homo sapiens
  source 1..83: mol_type = other DNA
cctttaaaaatttgccccgatgtaataaatatgcacatatcattacaccagttcgtccctttccagctttacagtgaattgcc

SEQ ID NO: 273 - DNA, 74 bp, Homo sapiens
  source 1..74: mol_type = other DNA
ggctgagggaactcaaagtacatgaacttgtcttcccgtcgtgtgggtcctgaattggaggaatatatcttcat

SEQ ID NO: 274 - DNA, 76 bp, Homo sapiens
  source 1..76: mol_type = other DNA
ggatcacaaagatttgtgattttggtctagccagagacatcaagaatgattctaattatgtggttaaaggaaacgc

SEQ ID NO: 275 - DNA, 91 bp, Homo sapiens
  source 1..91: mol_type = other DNA
ggtctatgtaaacataattgtttccatttatctcctcaacaaccttccactgtacttcatacatgggtttctgtggggagaaagggaaaac

SEQ ID NO: 276 - DNA, 68 bp, Homo sapiens
  source 1..68: mol_type = other DNA
gccacacattggagcatgccattcacgagcctgtcgtaagtcaggatttctggttttgctacaggaac

SEQ ID NO: 277 - DNA, 91 bp, Homo sapiens
  source 1..91: mol_type = other DNA
gaaactcacatcgaggatttccttgttggctttcggagatgttgcttctcttaattccttgatagcgacgggaattttaactttctcaccg

SEQ ID NO: 278 - DNA, 69 bp, Homo sapiens
  source 1..69: mol_type = other DNA
tgcatggtattctttctcttccgcacccagcagtttggccagcccaaaatctgtgatcttgacatgctt

SEQ ID NO: 279 - DNA, 51 bp, Homo sapiens
  source 1..51: mol_type = other DNA
gaggcagccgaagggcatgagctgcgtgatgagctgcacggtggaggtgag

SEQ ID NO: 280 - DNA, 47 bp, Homo sapiens
  source 1..47: mol_type = other DNA
gtggccgccaggtcttgatgtactcccctacagacgtgcgggtggtc

SEQ ID NO: 281 - DNA, 74 bp, Homo sapiens
  source 1..74: mol_type = other DNA
catgtgtagaaagcagatttctccatgattcatttgtatcttggagtaaaatatcatgaatcaagtatggaaga

SEQ ID NO: 282 - DNA, 62 bp, Homo sapiens
  source 1..62: mol_type = other DNA
ttcagcagtaggtgctttatttttaggtacttctcgcttggtttgagctgtttgaggaggtc

SEQ ID NO: 283 - DNA, 67 bp, Homo sapiens
  source 1..67: mol_type = other DNA
cagcttgaagagctcctccacagggatgccggggtacggggagccccccagcgtgaagatctcccat

SEQ ID NO: 284 - DNA, 56 bp, Homo sapiens
  source 1..56: mol_type = other DNA
cctgcaggatgggccggtgcggggagcgctctgtgggggcagatgacgctcaggga

SEQ ID NO: 285 - DNA, 60 bp, Homo sapiens
  source 1..60: mol_type = other DNA
ccaccccgtagctgaggatgcctgcatacacactgcccgcctcgtcagcctccaccagcg

SEQ ID NO: 286 - DNA, 71 bp, Homo sapiens
  source 1..71: mol_type = other DNA
gctggagacatctcacattgtttttgttgacgatcttgttgaagaagtcgttgacatatttgatagggaac

SEQ ID NO: 287 - DNA, 70 bp, Homo sapiens
  source 1..70: mol_type = other DNA
gctttgcacctgttttgttgtgtacactatagtattctttatcatacatgtctctggcaagaccaaaatt

SEQ ID NO: 288 - DNA, 72 bp, Homo sapiens
  source 1..72: mol_type = other DNA
cccatcaggtacttgccgatgagcttggcccgcttgcggcgcggctggtagatgacctcggtggagtcgata

SEQ ID NO: 289 - DNA, 76 bp, Homo sapiens
  source 1..76: mol_type = other DNA
ctgagtgtagatgatgtcatcctcgatgtcgaagaggtcctcgtcctcgtccgcgccgtgcaggtcctccaagtat

SEQ ID NO: 290 - DNA, 69 bp, Homo sapiens
  source 1..69: mol_type = other DNA
gctgaaaatgactgaatataaacttgtggtagttggagctggtggcgtaggcaagagtgccttgacgac

SEQ ID NO: 291 - DNA, 65 bp, Homo sapiens
  source 1..65: mol_type = other DNA
cagtcctcatgtactggtccctcattgcactgtactcctcttgacctgctgtgtcgagaatatcg

SEQ ID NO: 292 - DNA, 74 bp, Homo sapiens
  source 1..74: mol_type = other DNA
tcttgctggtgtgaaatgactgagtacaaactggtggtggttggagcaggtggtgttgggaaaagcgcactgat

SEQ ID NO: 293 - DNA, 69 bp, Homo sapiens
  source 1..69: mol_type = other DNA
tcgcctgtcctcatgtattggtctctcatggcactgtactcttcttgtccagctgtatccagtatgtca

SEQ ID NO: 294 - DNA, 73 bp, Homo sapiens
  source 1..73: mol_type = other DNA
cctgaggagcgatgacggaatataagctggtggtggtgggcgccggcggtgtgggcaagagtgcgctgaccac

SEQ ID NO: 295 - DNA, 55 bp, Homo sapiens
  source 1..55: mol_type = other DNA
atgtactggtcccgcatggcgctgtactcctcctggccggcggtatccaggatga

SEQ ID NO: 296 - DNA, 62 bp, Homo sapiens
  source 1..62: mol_type = other DNA
aggacttttagaattcttaaatgtcatccgcataggtgttttgtcaatattataaacaggat

SEQ ID NO: 297 - DNA, 75 bp, Homo sapiens
  source 1..75: mol_type = other DNA
tctagcttgctggtgtattcttcataggcctataaaataaagcagacttatattcaatccggactttgtcctgat

SEQ ID NO: 298 - DNA, 37 bp, synthetic construct
  misc_feature 1..37: note = synthetic construct
  source 1..37: mol_type = other DNA
agacgtgtgctcttccgatctgtgctgtgactgcttg

SEQ ID NO: 299 - DNA, 38 bp, synthetic construct
  misc_feature 1..38: note = synthetic construct
  source 1..38: mol_type = other DNA
agacgtgtgctcttccgatctagcacatgacggaggtt

SEQ ID NO: 300 - DNA, 42 bp, synthetic construct
  misc_feature 1..42: note = synthetic construct
  source 1..42: mol_type = other DNA
agacgtgtgctcttccgatctcaaatactccacacgcaaatt

SEQ ID NO: 301 - DNA, 42 bp, synthetic construct
  misc_feature 1..42: note = synthetic construct
  source 1..42: mol_type = other DNA
agacgtgtgctcttccgatctatttggatgacagaaacactt

SEQ ID NO: 302 - DNA, 41 bp, synthetic construct
  misc_feature 1..41: note = synthetic construct
  source 1..41: mol_type = other DNA
agacgtgtgctcttccgatcttgtgatgatggtgaggatgg

SEQ ID NO: 303 - DNA, 40 bp, synthetic construct
  misc_feature 1..40: note = synthetic construct
  source 1..40: mol_type = other DNA
agacgtgtgctcttccgatctgggacggaacagctttgag

SEQ ID NO: 304 - DNA, 44 bp, synthetic construct
  misc_feature 1..44: note = synthetic construct
  source 1..44: mol_type = other DNA
agacgtgtgctcttccgatctgcaatttctacacgagatcctct

SEQ ID NO: 305 - DNA, 48 bp, synthetic construct
  misc_feature 1..48: note = synthetic construct
  source 1..48: mol_type = other DNA
agacgtgtgctcttccgatctctttggagtatttcatgaaacaaatga

SEQ ID NO: 306 - DNA, 48 bp, synthetic construct
  misc_feature 1..48: note = synthetic construct
  source 1..48: mol_type = other DNA
agacgtgtgctcttccgatctacagtaaaaataggtgattttggtcta

SEQ ID NO: 307 - DNA, 41 bp, synthetic construct
  misc_feature 1..41: note = synthetic construct
  source 1..41: mol_type = other DNA
agacgtgtgctcttccgatctgcaactactggacgctggac

SEQ ID NO: 308 - DNA, 43 bp, synthetic construct
  misc_feature 1..43: note = synthetic construct
  source 1..43: mol_type = other DNA
agacgtgtgctcttccgatctcaattttgtttcaggacctgct

SEQ ID NO: 309 - DNA, 40 bp, synthetic construct
  misc_feature 1..40: note = synthetic construct
  source 1..40: mol_type = other DNA
agacgtgtgctcttccgatctggcagcaacagtcttacct

SEQ ID NO: 310 - DNA, 38 bp, synthetic construct
  misc_feature 1..38: note = synthetic construct
  source 1..38: mol_type = other DNA
agacgtgtgctcttccgatctcccagcttggaggctgc

SEQ ID NO: 311 - DNA, 39 bp, synthetic construct
  misc_feature 1..39: note = synthetic construct
  source 1..39: mol_type = other DNA
agacgtgtgctcttccgatctgccaggccgctgaagaca

SEQ ID NO: 312 - DNA, 45 bp, synthetic construct
  misc_feature 1..45: note = synthetic construct
  source 1..45: mol_type = other DNA
agacgtgtgctcttccgatctgcaattcactgtaaagctggaaag

SEQ ID NO: 313 - DNA, 47 bp, synthetic construct
  misc_feature 1..47: note = synthetic construct
  source 1..47: mol_type = other DNA
agacgtgtgctcttccgatctgaagatatattcctccaattcaggac

SEQ ID NO: 314 - DNA, 49 bp, synthetic construct
  misc_feature 1..49: note = synthetic construct
  source 1..49: mol_type = other DNA
agacgtgtgctcttccgatctcgtttcctttaaccacataattagaatc

SEQ ID NO: 315 - DNA, 40 bp, synthetic construct
  misc_feature 1..40: note = synthetic construct
  source 1..40: mol_type = other DNA
agacgtgtgctcttccgatcttttccctttctccccacag

SEQ ID NO: 316 - DNA, 43 bp, synthetic construct
  misc_feature 1..43: note = synthetic construct
  source 1..43: mol_type = other DNA
agacgtgtgctcttccgatcttcctgtagcaaaaccagaaatc

SEQ ID NO: 317 - DNA, 45 bp, synthetic construct
  misc_feature 1..45: note = synthetic construct
  source 1..45: mol_type = other DNA
agacgtgtgctcttccgatctggtgagaaagttaaaattcccgtc

SEQ ID NO: 318 - DNA, 45 bp, synthetic construct
  misc_feature 1..45: note = synthetic construct
  source 1..45: mol_type = other DNA
agacgtgtgctcttccgatctagcatgtcaagatcacagattttg

SEQ ID NO: 319 - DNA, 39 bp, synthetic construct
  misc_feature 1..39: note = synthetic construct
  source 1..39: mol_type = other DNA
agacgtgtgctcttccgatctcacctccaccgtgcagct

SEQ ID NO: 320 - DNA, 38 bp, synthetic construct
  misc_feature 1..38: note = synthetic construct
  source 1..38: mol_type = other DNA
agacgtgtgctcttccgatctaccacccgcacgtctgt

SEQ ID NO: 321 - DNA, 50 bp, synthetic construct
  misc_feature 1..50: note = synthetic construct
  source 1..50: mol_type = other DNA
agacgtgtgctcttccgatctcttccatacttgattcatgatattttact

SEQ ID NO: 322 - DNA, 42 bp, synthetic construct
  misc_feature 1..42: note = synthetic construct
  source 1..42: mol_type = other DNA
agacgtgtgctcttccgatctacctcctcaaacagctcaaac

SEQ ID NO: 323 - DNA, 40 bp, synthetic construct
  misc_feature 1..40: note = synthetic construct
  source 1..40: mol_type = other DNA
agacgtgtgctcttccgatcttgggagatcttcacgctgg

SEQ ID NO: 324 - DNA, 39 bp, synthetic construct
  misc_feature 1..39: note = synthetic construct
  source 1..39: mol_type = other DNA
agacgtgtgctcttccgatctccctgagcgtcatctgcc

SEQ ID NO: 325 - DNA, 39 bp, synthetic construct
  misc_feature 1..39: note = synthetic construct
  source 1..39: mol_type = other DNA
agacgtgtgctcttccgatctgctggtggaggctgacga

SEQ ID NO: 326 - DNA, 45 bp, synthetic construct
  misc_feature 1..45: note = synthetic construct
  source 1..45: mol_type = other DNA
agacgtgtgctcttccgatcttccctatcaaatatgtcaacgact

SEQ ID NO: 327 - DNA, 42 bp, synthetic construct
  misc_feature 1..42: note = synthetic construct
  source 1..42: mol_type = other DNA
agacgtgtgctcttccgatctattttggtcttgccagagaca

SEQ ID NO: 328 - DNA, 40 bp, synthetic construct
  misc_feature 1..40: note = synthetic construct
  source 1..40: mol_type = other DNA
agacgtgtgctcttccgatctatcgactccaccgaggtca

SEQ ID NO: 329 - DNA, 39 bp, synthetic construct
  misc_feature 1..39: note = synthetic construct
  source 1..39: mol_type = other DNA
agacgtgtgctcttccgatctacttggaggacctgcacg

SEQ ID NO: 330 - DNA, 40 bp, synthetic construct
  misc_feature 1..40: note = synthetic construct
  source 1..40: mol_type = other DNA
agacgtgtgctcttccgatctcgtcaaggcactcttgcct

SEQ ID NO: 331 - DNA, 42 bp, synthetic construct
  misc_feature 1..42: note = synthetic construct
  source 1..42: mol_type = other DNA
agacgtgtgctcttccgatctgatattctcgacacagcaggt

SEQ ID NO: 332 - DNA, 38 bp, synthetic construct
  misc_feature 1..38: note = synthetic construct
  source 1..38: mol_type = other DNA
agacgtgtgctcttccgatcttcagtgcgcttttccca

SEQ ID NO: 333 - DNA, 42 bp, synthetic construct
  misc_feature 1..42: note = synthetic construct
  source 1..42: mol_type = other DNA
agacgtgtgctcttccgatctgacatactggatacagctgga

SEQ ID NO: 334 - DNA, 40 bp, synthetic construct
  misc_feature 1..40: note = synthetic construct
  source 1..40: mol_type = other DNA
agacgtgtgctcttccgatctggtcagcgcactcttgccc

SEQ ID NO: 335 - DNA, 40 bp, synthetic construct
  misc_feature 1..40: note = synthetic construct
  source 1..40: mol_type = other DNA
agacgtgtgctcttccgatctcatcctggataccgccggc

SEQ ID NO: 336 - DNA, 48 bp, synthetic construct
  misc_feature 1..48: note = synthetic construct
  source 1..48: mol_type = other DNA
agacgtgtgctcttccgatctcctgtttataatattgacaaaacacct

SEQ ID NO: 337 - DNA, 41 bp, synthetic construct
  misc_feature 1..41: note = synthetic construct
  source 1..41: mol_type = other DNA
agacgtgtgctcttccgatctcaggacaaagtccggattga

SEQ ID NO: 338 - DNA, 103 bp, synthetic construct
  misc_feature 1..103: note = synthetic construct
  misc_difference 35..36: note = n is a, c, g, or t
  misc_difference 80..85: note = n is a, c, g, or t
  source 1..103: mol_type = other DNA
cgagacggatcaagcagaagacggcatacgagatnnacagctatgtgactggagttctagacgtgtgctcttccgatctnnnnnnctgtgctgtgactgcttg

SEQ ID NO: 339 - DNA, 39 bp, synthetic construct
  misc_feature 1..39: note = synthetic construct
  source 1..39: mol_type = other DNA
acccccgcccggcacccgcgtccgcgccatggccatcta

SEQ ID NO: 340 - DNA, 39 bp, synthetic construct
  misc_feature 1..39: note = synthetic construct
  source 1..39: mol_type = other DNA
aaccccgcccggcacccgcgtccgcgccatggccatcta

SEQ ID NO: 341 - DNA, 38 bp, synthetic construct
  misc_feature 1..38: note = synthetic construct
  source 1..38: mol_type = other DNA
acccccgcccgcacccgcgtccgcgccatggccatcta

SEQ ID NO: 342 - DNA, 38 bp, synthetic construct
  misc_feature 1..38: note = synthetic construct
  source 1..38: mol_type = other DNA
acccccgcccggcacccgcgtccgcgccatggccatca

SEQ ID NO: 343 - DNA, 39 bp, synthetic construct
  misc_feature 1..39: note = synthetic construct
  source 1..39: mol_type = other DNA
tcccccgcccggcacccgcgtccgcgccatggccatcta

SEQ ID NO: 344 - DNA, 38 bp, synthetic construct
  misc_feature 1..38: note = synthetic construct
  source 1..38: mol_type = other DNA
acccccgcccggcacccgcgtccgcgccatggcctcta

SEQ ID NO: 345 - DNA, 39 bp, synthetic construct
  misc_feature 1..39: note = synthetic construct
  source 1..39: mol_type = other DNA
acccgcgcccggcacccgcgtccgcgccatggccaacta

SEQ ID NO: 346 - DNA, 38 bp, synthetic construct
  misc_feature 1..38: note = synthetic construct
  source 1..38: mol_type = other DNA
acccccgccggcacccgcgtccgcgccatggccatcta

SEQ ID NO: 347 - DNA, 68 bp, Homo sapiens; identical to SEQ ID NO: 266
  source 1..68: mol_type = other DNA

SEQ ID NO: 348 - DNA, 16 bp, synthetic construct
  misc_feature 1..16: note = synthetic construct
  source 1..16: mol_type = other DNA
gagctttcactgtcgc

SEQ ID NO: 349 - DNA, 15 bp, synthetic construct
  misc_feature 1..15: note = synthetic construct
  source 1..15: mol_type = other DNA
ggccttcactgtcgc

SEQ ID NO: 350 - DNA, 16 bp, synthetic construct
  misc_feature 1..16: note = synthetic construct
  source 1..16: mol_type = other DNA
aagctttcactgtcgc

SEQ ID NO: 351 - DNA, 16 bp, synthetic construct
  misc_feature 1..16: note = synthetic construct
  source 1..16: mol_type = other DNA
gagatttcactgtcgc

SEQ ID NO: 352 - DNA, 16 bp, synthetic construct
  misc_feature 1..16: note = synthetic construct
  source 1..16: mol_type = other DNA
aagatttcactgtagc

SEQ ID NO: 353 - DNA, 15 bp, synthetic construct
  misc_feature 1..15: note = synthetic construct
  source 1..15: mol_type = other DNA
agctttcactgtcgc

SEQ ID NO: 354 - DNA, 16 bp, synthetic construct
  misc_feature 1..16: note = synthetic construct
  source 1..16: mol_type = other DNA
gagctctcactgtcgc

SEQ ID NO: 355 - DNA, 16 bp, synthetic construct
  misc_feature 1..16: note = synthetic construct
  source 1..16: mol_type = other DNA
gagctttcgctgtcgc

SEQ ID NO: 356 - DNA, 16 bp, synthetic construct
  misc_feature 1..16: note = synthetic construct
  source 1..16: mol_type = other DNA
gggctttcactgtcgc

SEQ ID NO: 357 - DNA, 16 bp, synthetic construct
  misc_feature 1..16: note = synthetic construct
  source 1..16: mol_type = other DNA
gagctttcactgtagc

SEQ ID NO: 358 - DNA, 65 bp, Homo sapiens; identical to SEQ ID NO: 291
  source 1..65: mol_type = other DNA

SEQ ID NO: 359 - DNA, 17 bp, synthetic construct
  misc_feature 1..17: note = synthetic construct
  source 1..17: mol_type = other DNA
gcactgtactcctcttg

SEQ ID NO: 360 - DNA, 17 bp, synthetic construct
  misc_feature 1..17: note = synthetic construct
  source 1..17: mol_type = other DNA
gcacagtactcgtcctg

SEQ ID NO: 361 - DNA, 17 bp, synthetic construct
  misc_feature 1..17: note = synthetic construct
  source 1..17: mol_type = other DNA
gcaccgtactcctcttg

SEQ ID NO: 362 - DNA, 17 bp, synthetic construct
  misc_feature 1..17: note = synthetic construct
  source 1..17: mol_type = other DNA
acactgtactcctcttg

SEQ ID NO: 363 - DNA, 17 bp, synthetic construct
  misc_feature 1..17: note = synthetic construct
  source 1..17: mol_type = other DNA
gcactgtactccccttg

SEQ ID NO: 364 - DNA, 17 bp, synthetic construct
  misc_feature 1..17: note = synthetic construct
  source 1..17: mol_type = other DNA
gcactgtactcctctcg

SEQ ID NO: 365 - DNA, 17 bp, synthetic construct
  misc_feature 1..17: note = synthetic construct
  source 1..17: mol_type = other DNA
gcactgtacccctcttg

SEQ ID NO: 366 - DNA, 16 bp, synthetic construct
  misc_feature 1..16: note = synthetic construct
  source 1..16: mol_type = other DNA
gcacagtctcgtcttg

SEQ ID NO: 367 - DNA, 17 bp, synthetic construct
  misc_feature 1..17: note = synthetic construct
  source 1..17: mol_type = other DNA
gtactgtactcctcttg

SEQ ID NO: 368 - DNA, 17 bp, synthetic construct
  misc_feature 1..17: note = synthetic construct
  source 1..17: mol_type = other DNA
gcactgtgctcctcttg

SEQ ID NO: 369 - DNA, 17 bp, synthetic construct
  misc_feature 1..17: note = synthetic construct
  source 1..17: mol_type = other DNA
gcattgtactcttcttg

SEQ ID NO: 370 - DNA, 17 bp, synthetic construct
  misc_feature 1..17: note = synthetic construct
  source 1..17: mol_type = other DNA
gcactgtattcctcttg